(19) United States
(12) Patent Application Publication
(10) Pub. No.: US 2011/0028399 A1
(43) Pub. Date: Feb. 3, 2011

(54) CITRULLINATED CYTOKINES

(75) Inventors: Tamara Loos, Beringen (BE); Paul Proost, Lubbeek (BE); Jozef Van Damme, Brussels (BE)

Correspondence Address: DINSMORE & SHOHL LLP, FIFTH THIRD CENTER, ONE SOUTH MAIN STREET, SUITE 1300, DAYTON, OH, 45402-2023, US

(73) Assignee: KATHOLIEKE UNIVERSITEIT LEUVEN, Leuven (BE)

(21) Appl. No.: 12/988,076

(22) PCT Filed: Apr. 15, 2009
(86) PCT No.: PCT/BE2009/000024

§ 371 Date: Oct. 15, 2010

(30) Foreign Application Priority Data
Apr. 15, 2008 (GB) 0806812.4

(51) Int. Cl. A61K 38/19; C07K 14/52; C07K 16/24; C12P 21/00; G01N 33/566; A61P 29/00

(52) U.S. Cl. 514/16.6; 530/300; 514/20.9; 530/389.2; 435/68.1; 435/7.1

(57) ABSTRACT

The present invention provides natural occurring, recombinant and synthetic chemokines, interleukins and cytokines in which at least one arginine residue is replaced by or modified into a cltrulline residue. The present Invention also relates to the use of said chemokines, interleukins or cytokines and pharmaceutical compositions comprising said chemokines, interleukins or cytokines as anti-inflammatory agents and as haematopoietic cell (including stem-cell, progenitor cell and leukocyte) or endothelial cell mobilizing agents. Furthermore, the present invention relates to the use of said chemokines, interleukins or cytokines to create antibodies and to use said chemokines, interleukins, cytokines and/or said antibodies as diagnostic tools and as a medicine. In addition, the present invention provides for the processes for the identification and production of the citrullinated chemokines, interleukins or cytokines of the invention.

### FIELD OF THE INVENTION

The present invention provides natural occurring, recombinant and synthetic chemokines, interleukins and cytokines in which at least one arginine residue is replaced by or modified into a citrulline residue. The present invention also relates to the use of said chemokines, interleukins or cytokines and pharmaceutical compositions comprising said chemokines, interleukins or cytokines as anti-inflammatory agents and as haematopoietic cell (including stem cell, progenitor cell and leukocyte) or endothelial cell mobilizing agents. Furthermore, the present invention relates to the use of said chemokines, interleukins or cytokines to create antibodies and to use said chemokines, interleukins, cytokines and/or said antibodies as diagnostic tools and as a medicine. In addition, the present invention provides for the processes for the identification and production of the citrullinated chemokines, interleukins or cytokines of the invention.

### BACKGROUND

Cytokines are a group of immune-mediators which amongst others comprise TNF-superfamily members, interleukins, chemokines. Chemokines are a family of small secreted proteins that activate and attract leukocytes during inflammation, but also play an important role in normal leukocyte-trafficking including lymphocyte homing. Chemokines exhibit high affinity for seven-transmembrane spanning G protein-coupled signaling receptors and matrix or cell bound glycosaminoglycans (GAG). These chemotactic cytokines contain conserved cysteine residues in their amino (NH₂)-terminal structure, a characteristic used for classification into CXC, CC, CX₃C and C chemokines. CXCL8 (interleukin-8/IL-8), that contains the tripeptide Glu-Leu-Arg (ELR) in front of the first Cys residue, is an inflammatory CXC chemokine with potent neutrophil chemotactic and angiogenic properties. CXCL8 and CXCL5 promote in vivo activation and recruitment of granulocytes through the chemokine receptors 1 and 2 (CXCR1 and CXCR2).

Chemokine activity is controlled at different levels, including regulation of chemokine and chemokine receptor expression, the presence of “silent” or “decoy” chemokine receptors, binding to GAG and posttranslational modification. Leukocytes have been reported to produce a mixture of proteolytically modified forms of CXCL8, derived from secreted intact CXCL8, i.e. CXCL8(1-77). Limited NH₂-terminal truncation by proteases such as thrombin, plasmin and metalloproteinases (MMPs) potentiates CXCL8 in vitro. However, cleavage in or beyond the ELR motive abrogates CXCL8 activity. In vivo, no significant difference in neutrophil accumulation or plasma protein exudation was observed between CXCL8(1-77) and CXCL8(6-77) upon intradermal injection in rabbits or intra-air pouch administration in mice. This apparent contradiction between in vitro and in vivo migration experiments may be explained by the rapid processing of CXCL8(1-77) in vivo as evidenced by studies with Mmp8-I-mice.

Chemokines exhibit redundancy in their binding and signaling capacities in that the ligands can interact with more than one chemokine receptor and vice versa. CXC chemokine ligand 10 (CXCL10) or interferon-gamma-inducible protein-10 (IP-10) is a potent attractant of lymphocytes and natural killer cells, recognized by CXC chemokine receptor 3 (CXCR3), a seven-transmembrane G-protein coupled receptor (GPCR). Interestingly, CXCL10 also exerts angiostatic properties, however, the exact mechanism is still unclarified. It was also suggested that CXCL10 binds to another receptor, yet unidentified, that is unable to bind other CXCR3 ligands, i.e. interferon T cell α-chemoattractant (I-TAC/CXCL11) and monokine induced by interferon-γ (Mig/CXCL9). CXCL11, the most potent CXCR3 ligand, on the other hand, was found to bind a second receptor, i.e. CXCR7. CXCR7 was reported to be expressed in various transformed cells and tumor development was diminished by treatment with a CXCR7 antagonist in mice inoculated with human lymphoma or carcinoma. Recently, a function in migration coordination was also appointed to CXCR7 in cooperation with CXCR4 in zebrafish.

Unlike most chemokines whose function and expression are specific and centered around their role in leukocyte trafficking, both SDF-1/CXCL12 (stromal derived factor-1/CXCL12) and its receptor CXCR4 were found to be expressed in a wide variety of cell types and tissues. Furthermore, this ubiquitous chemokine activates a broad spectrum of leukocytic target cells (including monocytes, lymphocytes, neutrophils and haematopoletic progenitor cells) and is hence an important mediator during infection, inflammation, haemotopoiesis, angiogenesis and tumor metastasis. Indeed, SDF-1 has been discovered rather as a cytokine, which promotes pre-B cell growth, before its chemotactic effect was elucidated. Importantly, mutant mice with a targeted description of the SDF-1 gene die perinatally. More recently, it was found that T-tropic (X4) HIV-infection request binding to a co-receptor, i.e. CXCR4 which is the major functional receptor for CXCL12. Very recently, however, a second receptor for CXCL12, i.e. CXCR7 or RDC1 has been identified, breaking up the monogamous relationship between CXCL12 and CXCR4. Although CXCL12 does not belong to the family of cytokine-inducible pro-inflammatory chemokines, several studies have revealed increased expression of CXCL12 in different models of inflammation. In particular, recent reports suggest that this chemokine may constitute a major determinant in RA development, inducing the recruitment of T cells, neo-vascularization of rheumatoid synovium and the release of MMP-3 by chondrocytes (Nanki, T et al. J. Immunol., 2000, 165, 6590-6598).

Several cytokines, chemokines and variants thereof have been described as (haematopoietic) stem cell mobilizing agents. Some of these show synergistic effects, as was shown for example with GRO-beta (CXCL2) in combination with G-CSF. Synergism was also shown for the combination of G-SCF with AMD3100, a CXCR4 antagonist.

Several chemokines and their receptors have been shown to be of importance in many severe diseases such as rheumatoid arthritis (RA), type I diabetes, multiple sclerosis and cancer. Furthermore, the regulation mechanisms are complex and multiple, including posttranslational modifications that can be fast and completely abrogate the biological activity. Therefore, the chemokines form a very interesting study object for discovering potential therapeutics or diagnostic applications. Today, there is a need for new, active and safer drugs or diagnostic tests for several diseases, including chemokine related diseases. The present invention fulfills these needs by analyzing new posttranslational modifications not described yet for chemokines or cytokines and analyzing the biological activity of these modified chemokines and cytokines in order to be of use in any treatment of such diseases.

### SUMMARY OF THE INVENTION

The present invention provides novel proteins selected from the group of cytokines and chemokines characterized in that said cytokines and chemokines have at least one of its Arg residues substituted by a citrulline residue (“citrullinated cytokines” and “citrullinated chemokines”). The present invention provides said citrullinated cytokines and chemokines for use as a medicine. Furthermore, the present invention also provides said citrullinated cytokines and chemokines to use for the mobilisation of haematopoietic cells (including stem cells, progenitor cells and leukocytes) or endothelial cells. In addition, the present invention also provides said citrullinated cytokines and chemokines to use for the prevention or treatment of inflammation and inflammation-related disorders. The present invention also provides pharmaceutical compositions that comprise said citrullinated cytokines or chemokines or variants or fragments thereof. Said pharmaceutical compositions may comprise citrullinated G-CSF, citrullinated GRO-beta, citrullinated CXCL12 peptide analogues such as CTCE-0021 and CTCE-0214. Said pharmaceutical compositions may further comprise uncitrullinated compounds such as G-CSF, GRO-beta, CXCL12 peptide analogues such as CTCE-0021 and CTCE-0214, and/or AMD3100. Furthermore, the present invention also provides said pharmaceutical compositions to use for the mobilisation of haematopoietic cells (including stem cells, progenitor cells and leukocytes) or endothelial cells. In addition, the present invention also provides said pharmaceutical compositions to use for the prevention or treatment of inflammation and inflammation-related disorders. The present invention provides also a method of treatment specifically directed against inflammation or inflammation-related disorders. The present invention also provides a process for the production of said citrullinated cytokines and chemokines. Furthermore, the present invention provides for antibodies to the citrullinated cytokines and chemokines of the invention. In addition, the present invention provides for a method of diagnosis using said antibodies of the invention to detect the citrullinated cytokines and chemokines of the invention.

One aspect of the present invention relates to a protein selected from the group of cytokines or chemokines characterized in that at least one of the arginine (Arg) residues of said chemokine is replaced by a citrulline residue, and variants, homologues or fragments thereof comprising said citrulline residue. Said citrulline residue can be in the L-form or D-form; preferentially said citrulline is L-citrulline. In a particular embodiment of the invention, said cytokine is selected from the group of inflammatory cytokines, interleukines, or TNF-superfamily members. In another particular embodiment of the invention, said cytokine is selected from the group of chemokines, inflammatory chemokines or constitutively expressed chemokines. In a particular embodiment of the invention, said cytokine is selected from the group of chemokines, preferably the CX₃C, CC, or C type chemokines; more preferably said cytokine is selected from the CXC type chemokines. In particular embodiments of the foregoing, said cytokine comprises the Glu-Leu-Arg (ELR) motif in front of the first NH₂-terminal Cys residue. In a particular embodiment of the foregoing, the first citrulline residue is located in the NH₂-terminal half of said cytokine, preferably in the NH₂-terminally first 20 amino acids of said cytokine, more preferably in the NH₂-terminally first 8 amino acids of said cytokine, even more preferably in the NH₂-terminally first 6 amino acids of said cytokine and most preferably in the NH₂-terminally first 5 amino acids of said cytokine. In a preferred embodiment of the foregoing, the first NH₂-terminally located Arg residue is replaced by a citrulline residue. In particular embodiments of the foregoing, said cytokine comprises maximum 10, 6 or 5 citrulline residues, particularly maximum 3 citrulline residues, more particularly maximum 2 citrulline residues, and even more preferably 1 citrulline residue. In particular embodiments of the invention, the 3 NH₂-terminally first Arg residues are replaced by citrulline residues, preferably the 2 NH₂-terminally first Arg residues are replaced by citrulline residues, and even more preferably the NH₂-terminally first Arg residue is replaced by a citrulline residue. In a particular embodiment of the invention, said chemokine or cytokine is selected from the group of citrullinated TNF-α, citrullinated IL-6, citrullinated CXCL8, citrullinated CXCL5, citrullinated CXCL9, citrullinated CXCL10, citrullinated CXCL11, citrullinated CCL17, citrullinated CCL14, citrullinated CCL26 and citrullinated CXCL12. In a more particular embodiment of the invention, said cytokine is TNF-αcit₂ in which the Arg at position 2 is replaced by a citrulline residue. In another particular embodiment of the invention, said cytokine is TNF-αcit_{2,6} in which the Arg residues at position 2 and 6 are replaced by a citrulline residue. In a more particular embodiment of the invention, said chemokine is CXCL8cit₅ in which the Arg at position 5 is replaced by a citrulline residue. In another embodiment of the invention, said chemokine is CXCL10cit₅ in which the Arg at position 5 is replaced by a citrulline residue. In another embodiment of the invention, said chemokine is CXCL11cit₆ in which the Arg at position 6 is replaced by a citrulline residue. In yet another embodiment of the invention, said chemokine is CXCL12cit₈ in which the Arg at position 8 is replaced by a citrulline residue. In yet another particular embodiment of the invention, said chemokine is CXCL12cit_{8,12,20} in which the Arginine residues at positions 8, 12, and 20 are replaced by citrulline residues. In yet another particular embodiment of the invention, said chemokine is CXCL12cit_{6, 12,20,41,47} in which the Arginine residues at positions 8, 12, 20, 41, and 47 are replaced by citrulline residues. In yet another particular embodiment of the invention, said chemokine is CCL14cit₆ in which the Arg at position 6 is replaced by a citrulline residue. In yet another particular embodiment of the invention, said chemokine is CCL26cit₂ in which the Arg at position 2 is replaced by a citrulline residue. In yet another particular embodiment of the invention, said chemokine is CXCL5cit₉ in which the Arg at position 9 is replaced by a citrulline residue. In yet another particular embodiment of the invention, said chemokine is CXCL9cit₅ in which the Arg at position 5 is replaced by a citrulline residue. In yet another particular embodiment of the invention, said chemokine is CCL17cit₂ in which the Arg at position 2 is replaced by a citrulline residue. in yet another particular embodiment of the invention, said chemokine is CCL17cit_{2,8} in which the Arg residues at position 2 and 8 are replaced by citrulline residues. In yet another particular embodiment of the invention, said chemokine is IL-6cit₁₅in which the Arg at position 15 is replaced by a citrulline residue.

Another aspect of the invention relates to a pharmaceutical composition comprising a pharmaceutically acceptable carrier and as an active ingredient, a therapeutically effective amount of a citrullinated chemokine or cytokine of the invention or a fragment thereof comprising said citrulline residue.

Another aspect of the invention relates to said citrullinated cytokines and chemokines of the invention and variants, homologues or fragments thereof comprising said citrulline residue for use as a research tool and to the use of said citrullinated cytokines and chemokines of the invention and variants, homologues or fragments thereof comprising said citrulline residue as a research tool.

Another aspect of the invention relates to said citrullinated cytokines and chemokines of the invention and variants, homologues or fragments thereof comprising said citrulline residue for use as a medicine and to the use of said citrullinated cytokines and chemokines of the invention and variants, homologues or fragments thereof comprising said citrulline residue as a medicine. A more particular embodiment of the invention relates to CXCL8cit₅ for use as a medicine. Another particular embodiment of the invention relates to CXCL10cit₅, CXCL11 cit₆, CXCL12cit₈, CXCL12cit_{6,12,20}, CXCL12cit_{8,12,20,41,47}, TNF-αcit₂, TNF-αcit_{2,6}, CCL14cit₆, CCL26cit₂, CXCL5cit₈, CXCL9cit₅, CCL17cit₂, CCL17cit_{2,8} and IL-6cit₁₅ for use as a medicine. A particular embodiment of the invention relates to the pharmaceutical composition of the invention for use as a medicine and to the use of said pharmaceutical composition as a medicine. Furthermore the invention relates to said citrullinated cytokines and chemokines of the invention and variants, homologues or fragments thereof comprising said citrulline residue to use for the mobilisation of haematopoietic cells (including stem cells, progenitor cells and leukocytes) or endothelial cells. A particular embodiment of the invention relates to the pharmaceutical composition of the invention to use for the mobilisation of haematopoietic cells (including stem cells, progenitor cells and leukocytes) or endothelial cells. A yet more particular embodiment of the invention relates to CXCL8cit₅ to use for the mobilisation of haematopoietic cells (including stem cells, progenitor cells and leukocytes) or endothelial cells. Yet another embodiment of the invention relates to said citrullinated cytokines and chemokines of the invention and variants, homologues or fragments thereof comprising said citrulline residue to use for the prevention or treatment of inflammation and inflammation-related disorders. A more particular embodiment of the invention relates to CXCL8cit₅, CXCL10cit₅, CXCL11cit₆, CXCL12cit₈, CXCL12cit_{6,12,20}, CXCL12cit_{8,12,20,41,47,} TNF-αcit₂, TNF-αcit_{2,6}, CCL14cit₆, CCL26cit₂, CXCL5cit₉, CXCL9cit₅, CCL17cit₂, CCL17cit_{2,6} and IL-6cit₁₅ to use for the prevention or treatment of inflammation and inflammation-related disorders. A particular embodiment of the invention relates to the pharmaceutical composition of the invention to use for the prevention or treatment of inflammation and inflammation-related disorders. In another particular embodiment, said prevention or treatment of inflammation and inflammation-related disorders is characterized in that it reduces the extravasation from the blood circulation of haematopoietic cells (including stem cells, progenitor cells and leukocytes) or endothelial cells.

Yet another aspect of the invention relates to the use of said citrullinated cytokines or chemokines of the invention and variants, homologues or fragments thereof comprising said citrulline residue or the pharmaceutical composition of the invention for the manufacture of a medicament. A particular embodiment of the invention relates to the use of said citrullinated cytokines or chemokines of the invention and variants, homologues or fragments thereof comprising said citrulline residue or the pharmaceutical composition of the invention for the manufacture of a medicament for the treatment or prevention of inflammation in a subject. Another particular embodiment of the invention relates to the use of said citrullinated cytokines or chemokines of the invention and variants, homologues or fragments thereof comprising said citrulline residue or the pharmaceutical composition of the invention for the manufacture of a medicament for the mobilisation of haematopoietic cells (including stem cells, progenitor cells and leukocytes) or endothelial cells in said subject.

Yet another aspect of the invention relates to an antibody specifically directed against said citrullinated cytokines or chemokines of the invention or variants, homologues or fragments thereof comprising said citrulline residue. A more particular embodiment of the invention relates to said antibodies of the invention for use as a medicine or as a diagnostic tool.

Another aspect of the present invention relates to a method for the mobilisation of haematopoietic cells (including stem cells, progenitor cells and leukocytes) or endothelial cells by administering the citrullinated cytokines or chemokines of the invention or variants, homologues or fragments thereof comprising said citrulline residue or the pharmaceutical composition of the invention to a subject in need. Another aspect of the invention relates to a method of treatment or repression of inflammation in a subject, by administering the citrullinated cytokines or chemokines of the invention or variants, homologues or fragments thereof comprising said citrulline residue or the pharmaceutical composition of the invention to said subject.

Another aspect of the present invention, relates to a method for diagnosing the presence of the citrullinated cytokines or chemokines of the invention by using the antibodies of the invention. A particular embodiment of the invention relates to a method for diagnosing the presence of the CXCL8cit₅, CXCL10citₛ, CXCL11cit₀, CXCL12citₛ, CXCL12cit_{0,12,20}, CXCL12cit_{0,12,20,41,47}, TNF-αcit₂, TNF-αcit_{2,6}, CCL14cit₆, CCL26cit₂, CXCL6cit₉, CXCL9cit₅, CCL17cit₂, CCL17cit_{2,5} and IL-6cit₁₅ by using the antibody directed against the respective cytokines or against variants, homologues or fragments thereof comprising said citrulline residue(s).

Another aspect of the present invention relates to a method to modulate the activity of cytokines or chemokines by substituting at least one Arg residue of the cytokine or chemokine by a citrulline residue. In a particular embodiment, the invention relates to a method to modulate the activity of cytokines or chemokines by substituting the first Arg residue starting from the NH₂-terminus of the chemokine or cytokine by a citrulline residue. In a particular embodiment of the foregoing, said chemokines are selected from the group of CXC-type chemokines. In another particular embodiment, said cytokines are selected from the group of CXCL8; CXCL10; CXCL11; CXCL12; CXCL5, CXCL9, CCL14, CCL17, CCL26, IL-6 and TNF-α.

Another aspect of the present invention relates to a process for the production of the citrullinated cytokines and chemokines of the invention or variants, homologues or fragments thereof by incubating the cytokines or chemokines or their variants, homologues or fragments with the enzyme peptidylarginine deiminase (PAD). Said PAD can be any PAD of any organism known in the art, including rabbit PAD and human PAD1, PAD2, PAD3, PAD4 and PAD6. Said process comprises two steps:
(i) the incubation of the cytokine or chemokine protein with the PAD enzyme, more particularly in a buffered solution, pH between pH 6 and 8, preferably between pH 7.2 and pH 7.6, and more preferably pH7.4, at a temperature between 30 and 40° C., preferably between 36.5 and 37.5° C., and more preferably at 37° C.; this solution may comprise a buffer, more preferably a TRIS-HCL buffered solution, preferably a buffered solution between 0 and 250 mM TRIS-HCL, preferably between 100 and 10 mM TRIS-HCL more preferably 40mM TRIS-HCl, and between 0.1 and 10 mM CaCl₂, preferably between 1 and 5 mM CaCl₂, more preferably 2 mM CaCl₂; and
(ii) stopping the deimination reaction, particularly by acidifying the reaction mixture, more particularly by adding between 0.05 and 1.5% acid, more preferably by adding 0.1% acid, said acid may be TFA.

Another embodiment of the invention relates to a process for the production of the citrullinated cytokines and chemokines of the invention or variants, homologues or fragments thereof by chemical synthesis. The citrullinated cytokines and chemokines of the invention or variants, homologues or fragments thereof can be generated using recombinant DNA techniques, in bacteria, yeast, insect cells, plant cells or mammalian cells, followed by an incubation process with PAD as described above.

Another way of production of said citrullinated cytokines and chemokines of the invention or variants, homologues or fragments thereof is by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains such as methylated cysteine and citrulline.

Chemical peptide synthesis methods are well described and peptides can be ordered from companies such as Applied Biosystems and other companies.

Peptide synthesis can be performed as either solid phase peptide synthesis (SPPS) or contrary to solution phase peptide synthesis. The best-known SPPS methods are t-Boc and Fmoc solid phase chemistry:

During peptide synthesis several protecting groups are used. For example hydroxyl and carboxyl functionalities are protected by t-butyl group, lysine and tryptophan are protected by t-Boc group, and asparagines, glutamine, cysteine and histidine are protected by trityl group, and arginine is protected by the pbf group. In particular embodiments, such protecting groups can be left on the peptide after synthesis.

Peptides can be linked to each other to form longer peptides using a ligation strategy (chemoselective coupling of two unprotected peptide fragments) as originally described by Kent (Schnolzer & Kent (1992) Int. J. Pept. Protein Res. 40, 180-193) and reviewed for example in Tam et al. (2001) Biopolymers 60, 194-205 provides the tremendous potential to achieve protein synthesis which is beyond the scope of SPPS. Many proteins with the size of 100-300 residues have been synthesised successfully by this method. Synthetic peptides have continued to play an ever increasing crucial role in the research fields of biochemistry, pharmacology, neurobiology, enzymology and molecular biology because of the enormous advances in the SPPS.

Alternatively, the peptides can be synthesised by using nucleic acid molecules which encode the uncitrullinated cytokines or chemokines of this invention in an appropriate expression vector which include the encoding nucleotide sequences, followed by an incubation process with PAD as described above. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

The physical and chemical properties of a peptide or protein of interest (e.g. solubility, stability) is examined to determine whether the peptide or protein is/would be suitable for use in therapeutic compositions. Typically this is optimised by adjusting the sequence of the peptide. Optionally, the peptide or protein can be modified after synthesis (chemical modifications e.g. adding/deleting functional groups) using techniques known in the art.

Yet another aspect of the present invention relates to a test kit for diagnosing the presence of the citrullinated cytokines or chemokines of the invention in patients comprising the antibodies of the invention. Yet another aspect of the present invention relates to a test kit for diagnosing the presence of antibodies against said citrullinated cytokines or chemokines of the invention in patients. In a particular embodiment of the invention said test kit for diagnosing the presence of antibodies against said citrullinated cytokines or chemokines of the invention comprises said citrullinated cytokines or chemokines of the invention or variants, homologues or fragments thereof comprising said citrulline residue. Said test kit may additionally comprise other conventional reagents such as buffers, substrates, wetting solutions and control cytokines or chemokines that are not citrullinated.

Another aspect of the present invention relates to a method for the determination of the predisposition of a patient to develop inflammation-related or autoimmune diseases like rheumatoid arthritis (RA), multiple sclerosis (MS), inflammatory bowel disease (IBD), atherosclerosis, asthma, sepsis, psorasis, and Alzheimer comprising the determination of the presence of any of the citrullinated cytokines or chemokines of the invention in a biological sample derived from said patient. In a particular embodiment, said biological sample is a fluid selected from the group consisting of blood, sputum, serum, plasma, saliva, tears, mucus, synovial and ascites fluid or said biological sample is a biopsy or tissue sample. In particular embodiments of the foregoing, said method is further characterized in that a test kit is used for determining the presence of said citrullinated cytokines or chemokines. In a particular embodiment, said test kit comprises specific antibodies against the citrullinated cytokines or chemokines of the invention. In particular embodiments of the foregoing, said test kit additionally comprises a solid phase onto which the specific antibodies are or can be bound. In other particular embodiments of the foregoing, said test kit additionally comprises a labeling group which is bound to the antibodies or can be bound thereto. In yet other particular embodiments of the foregoing, said test kit additionally comprises at least one other antibody class-specific test reagent. In a particular embodiment of the foregoing, the invention relates to said method for predicting responsiveness to a medicament.

In particular embodiments of the foregoing the subject or the patient is a mammal, more particularly a human being. Said human being can be a patient suffering from an autoimmune disease, a patient being suspected as having said autoimmune disease or at risk of developing said autoimmune disease. In a particular embodiment of the foregoing, said autoimmune disease is arthritis, more particularly rheumatoid arthritis (RA), multiple sclerosis (MS), inflammatory bowel disease (IBD), atherosclerosis, asthma, sepsis. In another particular embodiment said human being can be a patient suffering from inflammation related disorders.

### BRIEF DESCRIPTION OF THE FIGURES

FIG. 1 Purification and identification of natural citrullinated CXCL8. A. Natural human CXCL8 was purified from the culture medium of peripheral blood mononuclear cells that were stimulated with double stranded RNA and interferon-γ using heparin affinity, cation exchange and C8 RP-HPLC. Fractions containing CXCL8 immunoreactivity (as determined by ELISA) were subjected to Edman degradation and phenylthiohydantoin (PTH) derivates of the amino acids were identified by RP-HPLC. The top panel shows an overlay of the chromatograms of amino acids 3 to 5 in the amino acid (AA) sequence corresponding to the residues of a mixture of two CXCL8 forms, i.e. CXCL8(1-77)Cit₅ (amino acids Leu-Pro-Cit) and CXCL8(6-77) (amino acids Lys-Glu-Leu). The middle panel shows the chromatogram for a standard mixture containing 2 pmol of 19 different amino acids (standard one letter code for amino acids was used). Alternatively L-citrulline was applied on the reaction vessel of the protein sequencer and the elution profile for PTH-citrulline is shown in the lower panel.

B. Arginine Residues that are incorporated in Protein Sequences may be Converted into Citrulline by Peptidylarginine Deiminase (PAD).

FIG. 2 Modification of CXCL8 by peptidylarginine deiminase (PAD). Recombinant CXCL8 (5 μM) was incubated with rabbit PAD at an enzyme/substrate molar ratio (E/S) of 1/20 or 1/200 or with human PAD2 or human PAD4 at an E/S ratio of 1/200 for different time periods. Samples were applied on PVDF membranes for Edman degradation or in parallel were desalted on a C4 ZipTip prior to examination on an ion trap mass spectrometer to determine the presence of Arg (♦) or Cit (▪) at position 5. The % of Arg5 in the sequence or Cit5 was calculated both from the amount of PTH-Arg and PTH-Cit that were detected by RP-HPLC after Edman degradation for 5 cycles and from the percentage of converted protein detected by ion trap mass spectrometry.

FIG. 3 RP-HPLC purification of CXCL8 modified by peptidylarginine deiminase (PAD). Recombinant CXCL8 was incubated for 90 min with PAD at an enzyme/substrate molar ratio of 1/20, purified by C8 RP-HPLC and eluted in an acetonitrile gradient. Part of the column effluent (0.67%) was splitted on-line to an ion trap mass spectrometer and the averaged spectra for the chromatographic peaks were deconvoluted to obtain the Mr of the proteins (insert).

FIG. 4 In vitro neutrophil chemotactic activity of citrullinated CXCL8. The chemotactic activity of CXCL8(1-77), citrullinated CXCL8 (CXCL8(1-77)Cit₅) and CXCL8(6-77) for neutrophils was measured using a Boyden microchamber (4-6 independent experiments). The chemotactic index (±SEM) was calculated by dividing the number of migrated cells towards test samples by the number of spontaneously migrated cells towards buffer. Statistical analysis was performed using the Mann-Whitney test on paired values [* if p<0.05 for comparison with the corresponding concentration of CXCL8(6-77)].

FIG. 5 Calcium signaling capacity and receptor desensitization of citrullinated CXCL8 in neutrophils and HEK293 cells transfected with CXCR1 or CXCR2. The increase in intracellular calcium concentration ([Ca²⁺]ᵢ) in neutrophils and HEK293 cells transfected with CXCR1 or CXCR2 was measured using the ratiometric dye Fura-2. The cells were stimulated with CXCL8(1-77) (♦), CXCL8(6-77) (A▴) or citrullinated CXCL8, CXCL8(1-77)Cit₅ (▪). Values represent the mean (±SEM) increase of [Ca²⁺]ᵢ (nM) with a detection limit at 20 nM (dotted line) (neutrophils: n=5; CXCR1: n=3; CXCR2: n=5) (Panels A, B and C). Desensitization experiments were performed by re-challenging the cells with 5 nM of CXCL8(1-77) 100s after the first stimulus. Results (mean±SEM) represent the percentage inhibition of the second agonist by the first stimulus in comparison with buffer as the first stimulus (neutrophils: n=5; CXCR1: n=3; CXCR2: n=5) (Panels D, E and F). Significant differences were calculated using the Mann-Whitney test on paired values [* if p<0.05 for comparison between CXCL8(1-77) and CXCL8(1-77)Cit₅].

FIG. 6 In vitro phosphorylation of extracellular signal-regulated kinase 1 and 2 (ERK1/2) by CXCL8 forms in HEK293 cells transfected with CXCR2. The amount of phosphorylated ERK1/2 (pg phospho ERK1/2 per mg total protein) was measured by a specific ELISA after stimulation of serum-starved CXCR2-transfected HEK293 cells for 5, 10 or 20 min with 10 nM of CXCL8(1-77), CXCL8(6-77) or CXCL8(1-77)Cit₅. Results represent the percentage ERK1/2 phosphorylation (±SEM) compared to medium-treated cells (4 independent experiments). Statistical analysis was performed using the Mann-Whitney test on paired values [* if p<0.05 for comparison of CXCL8(1-77) with CXCL8(6-77); + if p<0.05 for comparison of CXCL8(1-77) with CXCL8Cit₅].

FIG. 7 Sensitivity of citrullinated CXCL8 to thrombin and plasmin cleavage. Recombinant CXCL8(1-77) and citrullinated CXCL8 were incubated with plasmin or thrombin for different time periods at an enzyme/substrate molar, ratio of 1/100. SDS-PAGE was performed under reducing conditions on Tris/tricine gels and proteins were visualized by silver staining. The bovine trypsin inhibitor (Mr 6,200) is visible as relative molecular mass marker indicated with arrows.

FIG. 8 Effect of citrullination on the receptor binding properties of CXCL8.

Increasing concentrations of unlabeled chemokine (CXCL8(1-77) (♦), CXCL8(6-77) (▴), CXCL8(1-77)Cit₅ (▪)) were added together with ¹²⁵I-CXCL8 to HEK-293 cells transfected with CXCR1 (panel A) or CXCR2 (panel B), neutrophils (panel C) or red blood cells (panel D). Results represent the mean percentage (±SEM) of remaining specific ¹²⁵I-CXCL8 binding (n=4 to n=6 for red blood cells or receptor transfected cells, n=2 for neutrophils). Statistical differences compared to CXCL8(1-77) were detected using the Mann-Whitney test (*** if p<0.001; ** if p<0.01; * if p<0.05).

FIG. 9 Effect of citrullination on the GAG binding properties of CXCL8. GAG binding was evaluated by immobilizing low molecular weight heparin or heparin sulphate on EpranEx plates. CXCL8 bound to the GAGs was detected with biotinylated anti-CXCL8 antibodies and peroxidase conjugated streptavidine. The optical density obtained without addition of CXCL8 and with addition of CXCL8 (1-77) were set to respectively, 0% and 100%. Results represent the mean % of specific chemokine binding (6 to 24 independent experiments)±SEM to heparin or heparan sulfate for human CXCL8 (1-77) (♦), CXCL8 (6-77) (▴) or CXCL8 (1-77)Cit₅ (▪). Statistical differences were detected using the Mann-Whitney test (*** if p<0.001, ** if p<0.01; * if p<0.05 for comparison of CXCL8 (1-77) with CXCL8 (1-77)Cit₅).

FIG. 10 Effect of citrullination on adhesion molecule expression on neutrophils.

Total blood was treated with varying concentrations of CXCL8(1-77), CXCL8(6-77) or CXCL8(1-77)Cit₅ for 10 min. The relative expression level of CD16 (panel A) and the relative number of CD11b+/CD16+ (panel B), CD16⁺/CD11b⁺(panel C) or CD15″/CD16⁺ (panel D) double positive cells compared to buffer treated cells (Co) was determined by FACS. Stimulation with 10⁻⁷ M fMLP was used as a positive control.

FIG. 11 In vivo angiogenic activity of truncated and citrulline CXCL8. Neovascularisation induced by CXCL8(1-77), CXCL8(1-77)Cit₅ and CXCL8(6-77) was tested in a corneal micropocket. Angiogenesis was scored daily (score 0 to 4) from days 4 to 8. The angiogenic index was calculated by dividing the maximal neovascularisation scores (occuring between days 5 and 7) with the spontaneous angionesis obtained by dilution buffer. The figure denotes the median (squares), the 25 to 75% range (boxes), the non-outlier range (wiskers) and extreme values (diamonds) acquired from 5 to 9 replicates (eyes). Statistical analysis was performed using the Mann-Whitney test [* if p<0.05 or ** if p<0.01 for comparison with CXCL8(6-77); § if p<0.05 or §§§ if p<0.001 for comparison with control].

FIG. 12 Effect of citrullination on extravasation of neutrophils in vivo. (Panels A and B) Induction of neutrophil infiltration was measured in NMRI mice by i.p. injection of 200 μpl vehicle (0.9% NaCl), CXCL8(1-77), CXCL8(6-77) or CXCL8(1-77)Cit₅. After 2 hours, the mice were sacrificed and the intraperitoneal cavity was washed with 5 ml of saline enriched with 2% FBS and 20 U/ml heparin. The total amount of leukocytes per μl peritoneal lavage solution was determined and cytospins were stained with Hemacolor solutions (Merck) for evaluation of the percentage of neutrophils by differential 100-cell counts. The figure denotes the median (squares), the 25 to 75% range (boxes), the non-outlier range (wiskers), extreme values (diamonds) and outliers (circles) acquired from 12-21 independent experiments. Statistical analysis was performed using the Mann-Whitney test [§§ if p<0.01 or §§§ if p<0.001 for increased values compared with vehicle alone].

FIG. 13 Effect of citrullination on granulocytosis induced by CXCL8 in vivo. Induction of leukocytosis was measured in New Zealand white rabbits (±3 kg) by i.v. injection (1 ml in PBS) of 10 μg of CXCL8(1-77) (♦), CXCL8(6-77) (▴) or CXCL8(1-77)Cit₅ (▪). Blood samples were collected by bleeding at a peripheral ear vein in potassium EDTA coated tubes. Total and differential leukocyte concentrations were determined 15 min pre-injection and at different time points post-injection.

Results represent the mean (±SEM) number of circulating granulocytes from three rabbits measured by counting the total amount of leukocytes and determining the percentage of granulocytes by differential cell counts on blood smears, both determined double blind by three researchers. Statistical analysis was performed using the Mann-Whitney test [* if p<0.05 or ** if p<0.01 for comparison between CXCL8(1-77) and CXCL8(6-77); ‡ if p<0.05 or ‡‡ if p<0.01 or ‡‡‡ if p<0.001 for comparison between CXCL8(1-77) and CXCL8(1-77)Cit₅; + if p<0.05 or ++ if p<0.01 or +++ if p<0.001 for comparison between CXCL8(6-77) and CXCL8(1-77)Cit₅]. Experiments were carried out in 2 rabbits when CXCL8 (6-77) was injected or 3 rabbits when CXCL8Cit₅ and CXCL8(1-77) was injected.

FIG. 14 Identification of naturally citrullinated CXCL10. Natural CXCL10 was subjected to Edman degradation. Overlays are shown of the RP-HPLC chromatograms detected at 270 nm (mAU, milli absorption units) of 2 pmol of the 19 PTH-amino acids (indicated by their one letter code on the upper chromatograms) and of the fifth amino acid in the CXCL10 sequence (A) or of PTH-citrulline (Cit) (B). The arrows in panels A and B indicate the major signal in these chromatograms.

FIG. 15 Modification of CXCL10 by peptidylarginine deiminase (PAD) and RP-HPLC purification of citrullinated CXCL10. Recombinant CXCL10 (100 pmol) was incubated with rabbit PAD4 (panel A), human PAD2 or PAD4 (panel B) at an enzyme-substrate molar ratio (E/S) of 1:20 or 1:200 for different time periods. Samples were applied on PVDF membranes for Edman degradation and in parallel were desalted on a C4 ZipTip prior to examination on an ion trap mass spectrometer to determine the presence of Arg (♦) or Cit (□) at position 5. The % conversion of Arg₅ into Cit₅ and the conservation of Arg₈ in the sequence was calculated from the amount of PTH-Arg and PTH-Cit that were detected by RP-HPLC after 5 and 8 cycles of Edman degradation and the Mᵣ detected by ion trap mass spectrometry.

(C) Recombinant CXCL10 was incubated with rabbit PAD for 90 min at an enzyme-substrate molar ratio of 1:20, purified by C8 RP-HPLC and eluted in an acetonitrile gradient and detected at 214 nm (mAU, milli absorption units). Part of the column effluent (0.67%) was splitted on-line to an ion trap mass spectrometer and the averaged spectra for the chromatographic peaks were deconvoluted to obtain the relative molecular mass (Mᵣ) of the proteins (insert indicated with arrow).

FIG. 16 In vitro biological activity of citrullinated CXCL10 in CHO cells transfected with CXCR3. (A) The chemotactic activity of CXCL10 and CXCL10-Cit₅ for CHO-CXCR3 cells was measured using a Boyden microchamber (5 or more independent experiments). (B) The amount of phosphorylated ERK1/2 or PKB/AKT (pg/mg total protein) was measured by specific ELISAs after stimulation of serum-starved CHO-CXCR3 cells for 5 min with CXCL10, CXCL10-Cit₅ or medium (control). Results represent the percentage ERK1/2 (□) and PKB/AKT (▪) phosphorylation (mean±SEM) compared to medium-treated cells (100%) (3 or more independent experiments). (C) The increase of [Ca²⁺]ᵢ in CHO-CXCR3 cells was measured upon stimulation with CXCL10 (♦) or CXCL10-Cit₅(□). Values represent the mean (±SEM) increase of [Ca²⁺]ᵢ of 3 or more independent experiments with a detection limit at 10 nM (-----) Desensitization experiments were performed by rechallenging the CHO-CXCR3 cells with 3 nM of CXCL10 100 s after the first stimulus. Results (mean±SEM) represent the percentage inhibition of the second agonist by the first stimulus in comparison with buffer as first stimulus. Significant differences were calculated using the Mann-Whitney test on paired values (* if p<0.05 or ** if p<0.01 for comparison with buffer; ‡ if p<0.05 or ‡‡ if p<0.01 for comparison of CXCL10 with CXCL10-Cit₅) for the corresponding chemokine concentration as depicted in panels A to D.

FIG. 17 In vitro biological activity of citrullinated CXCL11 in CXCR3 transfectants.

Phosphorylated ERK1/2 (A) or PKB/AKT (B) were measured by specific ELISAs after stimulation of serum-starved CHO-CXCR3 cells for 5 min with CXCL11, CXCL11-Cit₆ or medium (control). Results represent the percentage ERK1/2 (□) and PKB/AKT (▪) phosphorylation (mean±SEM) compared to medium-treated cells (100%) (3 or more independent experiments). Statistical analysis was performed using the Mann-Whitney test on paired values (* if p<0.05 or ** if p<0.01 or *** if p<0.001 for comparison with control). (C) The increase of [Ca^{2+]}ᵢ (nM) in U87-CXCR3 cells was measured upon stimulation with different doses (nM) of CXCL11 (♦) or CXCL11-Cit₆ (□). Values represent the mean (±SEM) increase of [Ca^{2+]}ᵢ (3 to 6 independent experiments) with a detection limit of 10 nM (------). Statistical analysis was performed using the Mann-Whitney test (‡ if p<0.05 or ‡‡ if p<0.01 for comparison of authentic with citrullinated chemokine).

FIG. 18 Effect of citrullination on the receptor binding properties of CXCL10 and CXCL11 on CXCR3 and CXCR7 transfectants and on PHA-activated T cells. (A) The receptor binding properties of CXCL10 (♦) and CXCL10-Cit₅ (□) on CHO-CXCR3 cells were determined by competition for ¹²⁵I-labeled CXCL10. Results represent the mean (±SEM) percentage ¹²⁵I-CXCL10 bound compared to the amount of bound ¹²⁵I-CXCL10 when no cold ligand was added (4 to 6 independent experiments). (B-D) The receptor binding properties of CXCL11 (♦) and CXCL11-Cit₆ (□) on CHO cells transfected with CXCR3 (B) or CXCR7 (C) and on PHA-activated T cells (D) were determined by competition for ¹²⁵I-labeled CXCL11. Results represent the mean (±SEM) percentage remaining ¹²⁵I-CXCL11 bound compared to the amount of bound ¹²⁵I-CXCL11 when no cold ligand was added (3 to 6 independent experiments).

FIG. 19 Effect of citrullination on the in vitro biological activity in T cells of CXCL10 and CXCL11.

(A,C) The chemotactic activity of CXCL10, CXCL10-Cit₅, recombinant (rec) CXCL11, synthetic (synth) CXCL11 and synthetic CXCL11-Cit₆ for PHA-activated T cells was measured using a Boyden microchamber (5 to 20 independent experiments). Statistical analysis was performed using the Mann-Whitney test on paired values (* if p<0.05 or ** if p<0.01 or *** if p<0.001 for comparison with buffer). (B,D) The increase of [Ca²⁺]ᵢ in PHA-activated T cells was measured upon stimulation with CXCL10 (♦) or CXCL10-Cit₅ (□) as shown in panel B or with CXCL11 (♦) or CXCL11-Cit₆ (□) as depicted in panel D. Values represent the mean (i SEM) increase of (Ca²⁺1; of three or more independent experiments with a detection limit at 5 nM (-------). Significant differences were calculated using the Mann-Whitney test on paired values (‡ if p<0.05 or ‡‡ if p<0.01 for comparison of authentic with citrullinated chemokine). (E) Desensitization experiments were performed by re-challenging the T cells with 1 nM of synthetic CXCL11 100 s after the first stimulus. Results (mean±SEM) represent the percentage inhibition of the second agonist by the first stimulus in comparison with buffer as first stimulus (3 to 5 independent experiments). Significant differences were calculated using the Mann-Whitney test (‡ if p<0.05 comparison between CXCL11 and CXCL11-Cit₆).

FIG. 20 Citrullinated CXCL10 and CXCL11 in heparin binding and wound healing.

(A-B) GAG binding was evaluated by immobilizing heparin on EpranEx plates, followed by adding a series of dilutions of CXCL10 (♦) or CXCL10-Cit₅ (□) (6 to 12 independent experiments as depicted in panel A or CXCL11 (♦) or CXCL11-Cit₆ (□) (3 to 6 independent experiments) as shown in panel B. GAG binding (mean±SEM) was detected by chemokine-specific biotinylated antibodies and shown as the percentage binding of 10 nM CXCL10 in panel A (SEM control was 1.07%) and of 100 nM CXCL11 in panel B (SEM control was 1.24%). Statistical analysis was performed using the Mann-Whitney test (‡ if p<0.05 or ‡‡ if p<0.01 or ‡‡‡ if p<0.001 for comparison of authentic with citrullinated chemokine). (C) Inhibition of endothelial cell migration was tested in an in vitro wound healing assay. A scar was drawn in a confluent monolayer of HMVEC and series of sample dilutions of CXCL11, CXCL11-Cit₆ and medium (control) were added to the cells. Differences in scar width were examined under a microscope before and after 24 h of treatment. And migration scores (mean±SEM) are shown. Statistical analysis was performed using the Mann-Whitney test (** if p<0.01; *** if p<0.001 compared to control).

FIG. 21 Conversion of CXCL12 by PAD. CXCL12 was treated with PAD at 1/20 E/S ratio for 5 to 45 min at 37° C. and the % conversion of Arg on position 8 (♦), 12 (▪) and 20 (▴) to Cit (Citrulline) was determined by Edman degradation.

FIG. 22 Synthesis of CXCL12 isoforms. CXCL12, CXCL12cit₈ and CXCL12cit_{8,12,20} were prepared by Fmoc solid phase peptide synthesis, folded and purified by RP-HPLC. CXCL12cit_{8,12,28,41,47} was prepared by incubating CXCL12 with PAD and subsequent folding and purification. Results show the deconvoluted mass spectra as determined by ion trap mass spectrometry of the four folded and purified proteins.

FIG. 23 Chemotactic activity of CXCL12 isoforms. CXCL12 (▪), CXCL12cit₈ (♦), CXCL12cit_{8,12,20} (▴) and CXCL12cit_{8,12,20,41,47} (o) were tested for their ability to induce a chemotactic response on monocytic THP-1 cells (upper panel) or PBMC (lower panel). Results represent the mean (±SEM) chemotactic index of 5 to 9 independent experiments. The non-parametric Mann Whitney U-test was used for statistical analysis (*=p<0.05; **=p<0.01 for a reduced chemotactic response compared to CXCL12).

FIG. 24 CXCR4 dependent calcium signalling of CXCL12 isoforms. CXCL12cit₆ (♦), CXCL12cit_{6.12.20} (▴), CXCL¹²cit_{8,12,20,41,47} (o) and native CXCL12 (▪) were compared for their ability to induce a rise in the [Ca²⁺]ᵢ in CHO-CXCR4 cells (upper Panel). Results represent the mean increase in [Ca^{2+]}ᵢ±SEM of four or more experiments. To test for the desensitising capacity of CXCL12 (Panel B), fura-2-loaded THP-1 cells were first stimulated with different concentrations of CXCL12 forms, followed by stimulation with 1 nM of CXCL12. The [Ca²⁺]ᵢ; increase induced by the second stimulus (1 nM of CXCL12) is shown. Results represent the mean increase in [Ca²⁺]ᵢ±SEM for four or more experiments.

FIG. 25 Phosphorylation of ERK or Akt/PKB by CXCL12 isoforms. The influence of CXCL12 (▪), CXCL12cit₆ (♦), CXCL¹²cit_{8,12,20} (▴) and CXCL12 cit_{8,12,20,41,47} (o) on the phosphorylation of ERK1 and ERK2 (upper panel) or Akt/PKB (Lower panel) was evaluated in CHO-CXCR4 cells. CHO-CXCR4 cells were treated with different concentrations of the CXCL12 forms for 5 minutes. Levels of phosphorylated ERK and Akt/PKB in the cell lysates were determined using specific ELISAs and the relative increase compared to medium-treated cells was calculated. Results represent the mean rise in phosphorylated ERK or Akt/PKB levels (±SEM) of three independent experiments. Statistical analysis was performed using the non-parametric Mann Whitney U-test (* p<0.05).

FIG. 26 Receptor binding properties of CXCL12 isoforms. Competition for binding of CXCL12^{AF647} to CXCR4- or CXCR7-transfected CHO cells was evaluated for CXCL12 (▪), CXCL12cit₈ (♦), CXCL12cit_{8,12,20} (▴) and CXCL¹²cit_{8,12,20,41,47} (o). Results are expressed as the % of remaining specific binding of 20 ng/m1 CXCL12^{AF647} to 200 μl of 3×10⁵ transfected CHO cells (mean±SEM of four or more independent experiments).

FIG. 27 Heparin binding properties of CXCL12 isoforms, CXCL12 (▪), CXCL12cit₈ (♦), CXCL12cit_{8,12,28} (▴) or CXCL¹²Cit_{8,12,20,41,47} (o) (100 μl; 0.3 nM to 100 nM) were allowed to interact with immobilized heparin and the amount of bound chemokine was detected with biotinylated anti-CXCL12 antibodies and peroxidase-conjugated streptavidine and TMB staining. Results represent the average relative OD of 9 experiments±SEM. The non-parametric Mann Whitney U-test was used for statistical analysis (**=p <0.01 or ***=p<0.001 for reduced heparin binding properties compared to CXCL12).

FIG. 28 Internalisation of CXCR4.

Freshly purified PBMC in preheated RPMI 1640+0.5% human serum albumin, were incubated with different concentrations of chemokine solutions or vehicle at 37° C. After 30 min 150 μl ice-cold PBS containing 2% fetal bovine serum (PACS buffer) with 15 μl PE labeled anti-human CXCR4 antibodies (BD Biosciences, San Jose, Calif., USA) or PE labeled isotype control antibodies (IgG_{2α′k}; BD Biosciences) was added to the cells. Subsequently, the cells were incubated at 4° C. for 30 minutes. After washing away unbound fluorescent label, the cells were fixed in fixation buffer and analyzed afterwards by flow cytometry. Discrimination between monocytes and lymphocytes was done by their difference in forward and side scattering. Results shown are % of remaining fluorescence (=% remaining receptors) on monocytes (upper panel) or lymphocytes (lower panel)±SEM. Fluorescence on isotype control treated samples was set at 100%. CXCL12 (▪); CXCL12cit₈ (♦); CXCL12cit_{8,12,20} (▴) and)CXCL12cit_{8,12,20,41,41}(o).

FIG. 29 Effect of citrullination on the antiviral activity of CXCL12.

Lymphocytic MT-4 cells were treated with varying concentrations of CXCL12 (▪), CXCL12Cit₈ (♦), CXCL12Cit_{8,12,20} (A) or CXCL12Cit_{8,12,20,41,47} (o) at the time of infection with the X4-using NL4.3 HIV-1 strain. At day 5 viral replication was determined in the culture supernatant with a commercial p24 Ag ELISA kit (PerkinElmer, Norwalk, Conn., USA).

### DETAILED DESCRIPTION OF THE INVENTION

### Definitions

As used herein, the singular forms “a”, “an” and “the” include plural referents unless the context clearly dictates otherwise.

“Cytokines” are a group of immune-mediators which comprise TNF-superfamily members, interleukins and chemokines. Another way of classification of cytokines is on their receptor binding properties. Five different groups of receptors (and their ligands) are distinguished:
(i) ligands that bind to immunoglobulin superfamily receptors: e.g., IL-1,M-CSF, C-kit, IL18;
(ii) ligands that bind to class I cytokine receptors (hematopoietin): e.g. IL-2, IL-3, IL-4, IL-5, IL-6, IL-7, IL-9, IL-11, IL12, IL-13, IL-15, IL-21, IL-23, IL-27, GM-CSF, G-CSF, OSM, LIF, CNTF, Growth hormone, Prolactin;
(iii) ligands that bind to class II cytokine receptors (interferon): IFN-α, IFN-β, IFN-γ, IL-10, IL-19, IL-20, IL-22, IL-24, IL-26, IL-28, IL-29;
(iv) ligands that bind to TNF receptors: TNF-α, TNF-β, CD27L, CD30L, CD40L, Nerve growth factor (NGF), FAS; and
(v) ligands that bind to chemokine receptors: IL-8, RANTES, MIP-1, PF4, MCAF and NAP-2.

“Chemokines” are a family of structurally related glycoproteins with potent leukocyte activation and/or chemotactic activity. They are normally around 70 to 90 amino acids in length and approximately 8 to 10 kDa in molecular weight. Most of them fit into. two subfamilies with four cysteine residues. These subfamilies are based on whether the two amino terminal cysteine residues are immediately adjacent or separated by one amino acid. The CXC chemokines, contain a single amino acid between the first and second cysteine residues; while the CC chemokines have adjacent cysteine residues. Most CXC chemokines are chemoattractants for neutrophils whereas CC chemokines generally attract monocytes, lymphocytes, basophils, and/or eosinophils. There are also 2 other small sub-groups. The C group has one member (lymphotactin). It lacks two of the cysteines in the four-cysteine motif, but shares homology at its carboxyl terminus with the CC chemokines. The C chemokine seems to be lymphocyte specific. The fourth subgroup is the CX₃C subgroup. The CX₃C chemokine (fractalkine/neurotactin) has three amino acid residues between the first two cysteines. It is tethered directly to the cell membrane via a long mucin stalk and induces both adhesion and migration of leukocytes.

The CXC chemokines is a group of chemokines that currently has 17 members: CXC chemokine ligand (CXCL)-1 to CXCL-17. Some of these CXC chemokines have an additional specific amino acid sequence (or motif) of Glutamic acid-Leucine-Arginine (or ELR for short) immediately before the first cysteine of the CXC motif (ELR-positive), and those without an ELR motif (ELR-negative). ELR-positive CXC chemokines specifically induce the migration of neutrophils, and interact with chemokine receptors CXCR1 and CXCR2. An example of an ELR-positive CXC chemokine is CXCL8 also named interleukin-8 (IL-8), which induces neutrophils to leave the bloodstream and enter into the surrounding tissue. Other examples of ELR-positive CXC chemokines are CXCL1 and CXCL2, CXCL2 is also known as GRO-beta. Other CXC chemokines that lack the ELR motif, such as CXCL13, tend to be chemoattractant for lymphocytes. CXC chemokines bind to CXC chemokine receptors, of which seven have been discovered to date, designated CXCR1-7.

For most chemokines, different names are used, mostly referring to their function or origin. Examples include, but are not limited to “CXCL10”, which is also known as interferon-gamma-inducible protein 10 (IP-10); “CXCL11” refers to the well-known chemokine, also named IFN-inducible T-cell α-chemoattractant (I-TAC); “CXCL12” which is also known as stromal cell-derived factor-1 (SDF-1), “CXCL5” refers to epithelial cell-derived neutrophil-activating protein-78 (ENA-78), “CXCL9” refers to monokine induced by interferon-γ (Mig), “CCL14” refers to hemofiltrate CC chemokine-1 (HCC-1), CCL17 refers to thymus- and activation-regulated chemokine (TARC) and CCL26 refers to eotaxin-3.

The term “citrullinated cytokine” refers to a cytokine characterized in that said cytokine has at least one of its Arg residues substituted by a citrulline residue. The term “citrullinated chemokine” refers to a chemokine characterized in that said chemokine has at least one of its Arg residues substituted by a citrulline residue. “Substituted by” or “replaced by” does include modified, eg. an Arg residue that is substituted or replaced by a citrulline residue can also mean an Arg residue modified into a citrulline residue, e.g. by incubation with PAD. Citrullination by PAD starts mostly at the NH₂-terminus of the protein, but exceptionally it can start from the COOH terminus of the protein. In case several Arg residues are replaced by citrulline residues, this means that for said Arg residues each single Arg residue is replaced by one single citrulline residue.

The term “TNF superfamily” refers to a family of ligands that bind to TNF receptors. Examples of ligands belonging to the TNF superfamily include TNF-α, TNF-β, CD27L, CD30L, CD40L, Nerve growth factor (NOF) and FAS.

The term “interleukins” refers to secreted regulatory proteins of the immune systems designated as interleukins according to the criteria as recommended by the International Union of Immunological Societies (IUIS) (Paul W E et al. Clin. exp. Immunol., 1992,88:367).

The term “peptide” as used herein refers to a molecule comprising an amino acid sequence of between 2 and 200 amino acids, connected by peptide bonds. Peptides can contain any of the conventional 20 amino acids or modified versions thereof, or can contain non-naturally occurring amino-acids incorporated by chemical peptide synthesis or by chemical or enzymatic modification.

The term “homologue” as used herein with reference to a certain chemokine or cytokine refers to molecules having at least 50%, more preferably at least 70%, yet more preferably 80%, still more preferably 90%, again more preferable 95% and most preferably at least 98% amino acid sequence identity with said chemokine or said cytokine.

The term “derivative”, “variant” and “fragment” as used herein with reference to certain citrullinated chemokines or cytokines of the invention refers to molecules which comprises at least the active portion, comprising at least said citrulline residue of said chemokine or said cytokine and, in addition thereto comprises a complementary portion which can have different purposes such as stabilising the peptide or altering the pharmacokinetic or pharmacodynamic properties of the peptide.

The term “therapeutically effective amount” is meant an amount of a certain cytokine or chemokirie of the invention a fragment, derivative, variant or homologue thereof or modulators of the activity of a cytokine or chemokine, which produces the desired therapeutic effect in a patient. For example, in reference to a disease or disorder, It is the amount which reduces to some extent one or more symptoms of the disease or disorder, and preferably returns to normal, either partially or completely, the physiological or biochemical parameters associated or causative of the disease or disorder. Preferably, according to one aspect of the present invention, the therapeutically effective amount is the amount of certain cytokines or chemokines of the invention or modulators which will lead to an improvement of Inflammation related disorders such as RA, MS or IBD.

The term “antibody” as used herein refers to IgG, IgM, IgD, IgA and IgE antibody. The definition includes monoclonal and polyclonal antibodies and also includes nanobodies or the like from other organisms or mammals (i.e. camel antibodies). This term refers to whole antibodies or fragments or derivatives of the antibodies. The term “antibody fragment” refers to a sub-part of an antibody which alone, or in combination with other fragments, is capable of binding to the antigen against which it was raised. Typical antibody fragments are Fab, Fab′, F(ab′)₂, Fv or scFv, which often retain affinity for the antigen which is comparable to the complete antibody. Smaller fragments include complementarity determining regions or CDRs such as CDR1, CDR2 and CDR3 of the heavy or light chain and/or combinations of two or more thereof. The term “derivative” of an antibody or antibody fragment is used herein to refer to an antibody or antibody fragment which is the result of a modification of the original antibody (e.g. as produced by a hybridoma cell line) e.g. with respect to its amino acid sequence (e.g. for humanization, increasing the affinity to the antigen or binding to other molecules such as labels), but without significantly affecting the binding of the antibody or fragment to the antigen. Derivatives include alternative structures of one or more CDRs resulting in an antigen-binding molecule such as a synthetic polypeptide. A derivative of an antibody or antibody fragment as used herein refers also to any antibody or other antigen-binding molecule which can be directly derived from the antibody or a fragment thereof, while retaining the ability to bind their original antigen. Thus, derivatives include but are not limited to humanized versions of antibodies, including hybrid antibodies and antibodies or other antigen-binding molecules which have been obtained by grafting or introducing one or more of the variable regions and/or CDRs of such antibodies. Thus derivatives include mouse and human antibodies as well as antibodies obtained from other species, such as but not limited to camelid antibodies or nanobodies obtained therefrom. Additionally the term ‘derivatives’ of an antibody or antibody fragment includes antibodies and antibody fragments which have been modified with respect to glycosylation. A “humanized antibody or humanized antibody fragment” is also covered by the term antibody and refers to an antibody or fragment thereof in which amino acids have been replaced in order to more closely resemble a human antibody. The majority of these substitutions will be in the non-antigen binding regions. However, it is envisaged that within the CDRs, amino acids which do not or hardly take part in the binding to the antigen can also be substituted. A “Reshaped” antibody or antibody fragment or a “hybrid antibody” are also comprised in the term antibody as used herein and refers to an antibody which comprises parts of at least two antibodies of a different antigen. Typically, a human hybrid antibody can be a human constant region linked to a humanized variable region of another antibody directed against the antigen of interest or a human antibody backbone in which amino acid sequences in the antigen binding regions have been replaced with sequences from another antibody e.g. directed against a human antigen of interest. More particularly the antigen-binding regions of an antibody having an affinity for an antigen of interest, such as one or more CDRs or variable regions or parts thereof are introduced into the backbone of a human antibody (e.g. CDR-grafted antibodies). Reshaped or hybrid antibodies can thus have affinities for two different antigens or epitopes of one antigen.

The term “subject” or “patient” refers to any human, animal, mammals.

The term “autoimmune disease” (AD) refers to a disease that result from an aberrant immune response of an organism against its own cells and tissues due to a failure of the organism to recognise its own constituent parts (down to the sub-molecular level) as “self”. As used herein, “autoimmune disease” is intended to further include autoimmune conditions, syndromes and the like. Examples of autoimmune diseases include, but are not limited to rheumatoid arthritis (RA), spondylarthritis, ankylosing spondylitis, psoriatic arthritis, multiple sclerosis (MS), psoriasis, Alzheimer disease, Crohns' disease, inflammatory bowel disease (IBS), atherosclerosis, asthma, sepsis, nepropathy (e.g. diabetic nephropathy), glomerulonephritis, autoimmune hepatitis, Periodontitis, type 1 diabetes, myasthenia gravis, systemic lupus erythematosus, Sjogren Syndrome, adrenalitis, atherosclerosis and mixed connective tissue disease.

The term “inflammation related disorders” refers to diseases that induce extravasation or activation of inflammatory leukocytes, i.e. neutrophil granulocytes, eosinophil granulocytes, basophil granulocytes, monocytes, macrophages, dendritic cells, natural killer (NK) cells, activated T lymphocytes or activated B lymphocytes. Examples of inflammation related disorders include, but are not limited to RA, Crohn's disease, psoriasis, meningitis and asthma.

Patients suffering from inflammation related and autoimmune diseases including but not limited to, rheumatoid arthritis, insulin dependent diabetes mellitus, hemolytic anemias, rheumatic fever, thyroiditis (e.g. Hashimoto's thyroiditis), Crohn's disease, myasthenia gravis, glomerulonephritis, autoimmune hepatitis, multiple sclerosis, systemic lupus erythematosus and others, are in need of treatment in accordance with the present invention. Patients suffering from inflammation related and autoimmune diseases wherein PAD is misexpressed including but not limited to rheumatoid arthritis (RA), spondylarthritis, ankylosing spondylitis, psoriatic arthritis, multiple sclerosis (MS), psoriasis, Alzheimer disease, Crohns' disease, IBS, nepropathy, hepatitis and Periodontitis are in need of treatment in accordance with the present invention.

Treatment of patients suffering from these diseases by administration of the citrullinated cytokines or chemokines or fragments, homologues or variants thereof comprising said citrulline residue(s) in accordance with the present invention will alleviate the clinical manifestations of the disease and/or minimize or prevent further deterioration or worsening of the patient's condition. Treatment of a patient at an early stage of an autoimmune disease including, rheumatoid arthritis, insulin-dependent diabetes mellitus, multiple sclerosis, myasthenia gravis, systemic lupus erythematosus and others, will minimize or eliminate deterioration of the disease state into a more serious condition.

The term “mobilisation of haematopoietic cells (including stem cells, progenitor cells and leukocytes) or endothelial cells” refers to various blood or bone marrow cells or hematopoietic stem cell mobilisation from the bone marrow to the bloodstream or from the blood circulation to the tissues. In example 1 this is measured as the differential amount of granulocytes (neutrophils, eosinophils, and basophils) present in the bloodstream or peritoneal cavity before and after said mobilisation by intravenous or intraperitoneal injection.

The term “antigen” as used herein refers to a structure of a macromolecule, typically protein (with or without polysaccharides) or made of proteic composition comprising one or more hapten(s) and/or comprising at least one epitope.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B or T cell lymphocyte, and which is able, by said binding, to induce an immune response. An epitope can comprise as few as 3 amino acids in a spatial conformation which is unique to the epitope. Generally an epitope consists of at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by H. Mario Geysen et al. 1984. Proc. Natl. Acad. Sci. U.S.A. 81:3998-4002; PCT Publication^{.} No. WO 84/03564; and PCT Publication No. WO 84/03506.

The phrase “specifically (or selectively) binds” to an antibody, when referring to a protein or peptide, refers to a binding reaction that is determinative of the presence of the protein in a heterogeneous population of proteins and other biologics. The term “directed against” a protein or peptide, when referring to an antibody, refers also to the specific binding reaction that is determinative of the presence of the protein by said antibody in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Immunoassay” is an assay that uses an antibody to specifically bind an antigen (e.g. a marker). The immunoassay is characterized by the use of specific binding properties of a particular antibody to isolate, target, and/or quantify the antigen.

“Diagnostic” means identifying the presence or nature of a pathologic condition or presence of one or more citrullinated cytokines, chemokines or antibodies of the invention. The diagnostic method makes the correlation between the presence of citrullinated cytokines or chemokines and the occurrence of a specific disease or a group of diseases (e.g. autoimmunity).

Amino acids are referred to herein with their full name, their three letter abbreviation or their one letter abbreviation. As an example R and Arg stand for Arginine.

“Citrulline” and “Cit” refers to 2-amino-5-(carbamoylamino)pentanoic acid and is an alfa-amino acid with formula: H₂NC(O)NH(CH₂)₃CH(NH₂)CO₂H.

The term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the monoclonal antibody active ingredient in the composition. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidyiglycerine, lysolecithin, cardiolipin, dioctanyiphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 8 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol—polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbucw”, 2 d ed. (Hanser Verlag, Vienna, 1981) and “Encyclopaedia of Surfactants, (Chemical Publishing Co., New York, 1981).

The citrullinated cytokines or chemokines of the invention, fragments, derivatives, variants or homologues thereof comprising said citrulline residue according to the invention (and their physiologically acceptable salts, all included in the term “active ingredients”) and their antibodies may be administered by any route appropriate to the condition to be treated and appropriate for the proteins and fragments to be administered. Possible routes include regional, systemical, oral, rectal, nasal, topical (including ocular, buccal and sublingual), vaginal and parenteral (including subcutaneous, intramuscular, intravenous, intradermal, intraarterial, intrathecal and epidural). The preferred route of administration may vary with for example the condition of the recipient or with the diseases to be treated. The preferred way of administration for the mobilisation of haematopoietic cells (including stem cells, progenitor cells and leukocytes) or endothelial cells is intravenous. Regional/local treatment is useful for treatment of inflammation related disorders in a patient, including, but not limited to autoimmune diseases.

As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste. A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

For local treatments for example on the skin, such as of the joint, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules. and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

The citrullinated cytokines or chemokines of the invention, fragments, derivatives, variants or homologues thereof comprising said citrulline residue and their antibodies according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polyniethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

Monoclonal antibodies against the citrullinated chemokines of the invention or against fragments, derivatives, variants or homologues thereof comprising said citrulline residue can be produced by any technique which provides the production of antibodies by continuous cell lines in cultures such as the hybridoma technique originally developed by Kohler and Milstein (Kohler and Milstein Nature 1975, 256: 495-497), as well as the trioma technique, the human B-cell hybridoma technique (Kozbor et al., Immunology Today 1983, 4: 72), the EBV-hybridoma technique to produce human monoclonal antibodies (Cole et al. 1985, in “Monoclonal Antibodies and Cancer Therapy,” Alan R. Liss, Inc. Pp 77-96) and the like, all are within the scope of the present invention.

The monoclonal antibodies against the citrullinated chemokines of the invention or against fragments, derivatives, variants or homologues thereof comprising said citrulline residue, may be human monoclonal antibodies or chimeric human-mouse (or other species) monoclonal antibodies or even from any other kind known in the art, such as coming from cammels or lamas. Human monoclonal antibodies may be made of any numerous techniques known in the art (e.g. Teng et al, Proc. Natl. Acad. Sci. U.S.A. 1983, 80: 7308-7312; Kozbor et al., Immunology Today 1983, 4: 72-79, Olsson et al, Meth. Enzymol. 1982, 92: 3-16). Chimeric antibodies may be prepared containing a mouse antigen-binding domain with human constant regions (Morrison et al, Proc. Natl. Acad. Sci. U.S.A. 1994, 81: 6851, Takeda et al. Nature 1985, 314: 452).

Various procedures known in the art may be used for the production of polyclonal antibodies to epitopes of citrullinated chemokines of the invention. For the production of antibodies, various host animals can be immunized by injection with a specific CD13 protein or related protein, or a fragment or derivative thereof, including but not limited to rabbits, mice and rats. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenols, and potentially useful human adjuvants such as BCG (Bacille Calmette-Guerin) and Corynebacterium parvum.

A molecular clone of an antibody to a selected citrullinated chemokine epitope or related protein epitope can be prepared by known techniques. Recombinant DNA methodology (see e.g. Maniatis et al., 1982, Molecular Cloning, A Laboratory Manual, Cold Spring Harbor Laboratory, New York) may be used to construct nucleic acid sequences which encode a monoclonal antibody, or antigen binding region thereof.

The present invention provides for antibodies as well as fragments of such antibodies. Antibody fragments, which contain the idiotype of the molecule, can be generated by known techniques. For example, such fragments include but are not limited to the F(ab′)2 fragment which can be produced by pepsin digestion of the antibody, the Fab′ fragments which can be generated by reducing the disulfide bridges of the F(ab′)2 fragment and the Fab fragments which can be generated by treating the antibody with papain and a reducing agent. Antibodies can be purified by known techniques, e.g. immunoabsorption or immunoaffinity chromatography, chromatographic methods such as HPLC (high performance liquid chromatography), or a combination thereof.

### DETAILED DESCRIPTION

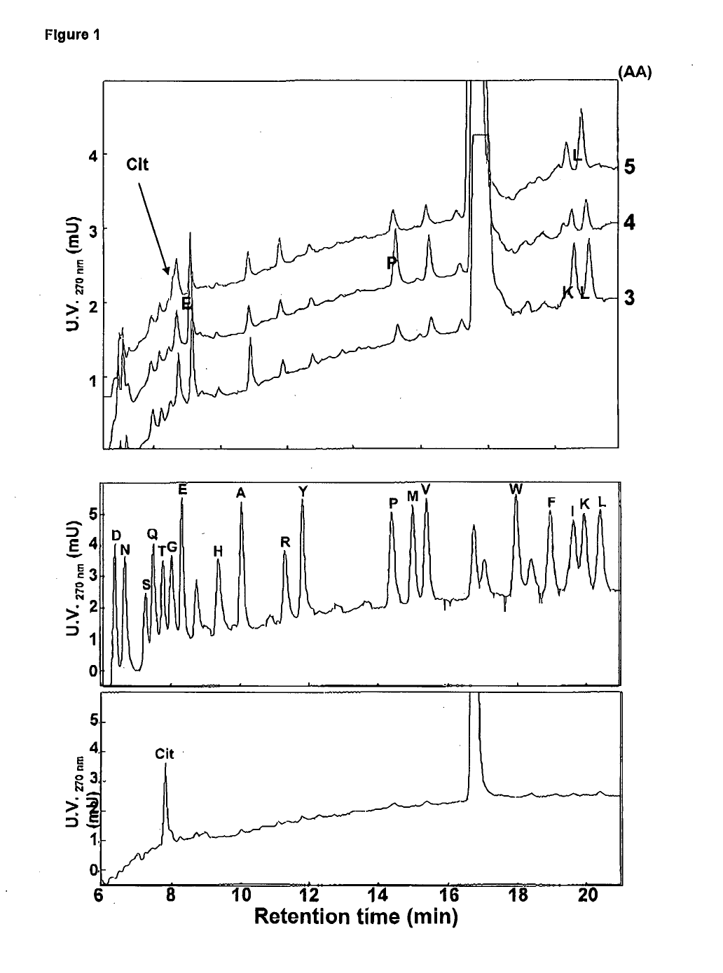

During the past decade posttranslational modification of chemokines has been reported to affect their in vitro and in vivo activities. Primarily NH₂-terminal processing alters the receptor affinity and specific biological activity of chemokines. This includes minimal modification of an NH₂-terminal Gln to pyroglutamic acid in the three monocyte chemotactic proteins MCP-1/CCL2, MCP-2/CCL8 and MCP-3/CCL7. For MCPs, this pyroglutamic acid is essential for full biological activity. Proteolytic processing of the NH₂-terminus of chemokines results in enhanced or reduced activity depending on the chemokine, protease and degree of processing involved. In addition to the numerous naturally occurring forms of NH₂-terminally truncated chemokines, a limited number of COOH-terminally processed chemokines have been identified (e.g. CCL2, CXCL7 and CXCL10). Some chemokines, e.g. CCL2 and CCU 1, may also be glycosylated. Despite the significant increase in relative molecular mass, glycosylation only moderately (two-fold) influences the in vitro activities of natural CCL2. NH₂-terminal truncation of CXCL8 by thrombin, plasmin, MMP-8 or MMP-9 by 5 to 8 amino acids has been reported to significantly increase its in vitro receptor signaling and chemotactic activity. In addition to NH₂-terminal proteolytic processing, the present invention reveals a novel and biologically relevant enzymatic modification of Arg into Cit in position five of leukocyte-derived CXCL8 (FIG. 1).

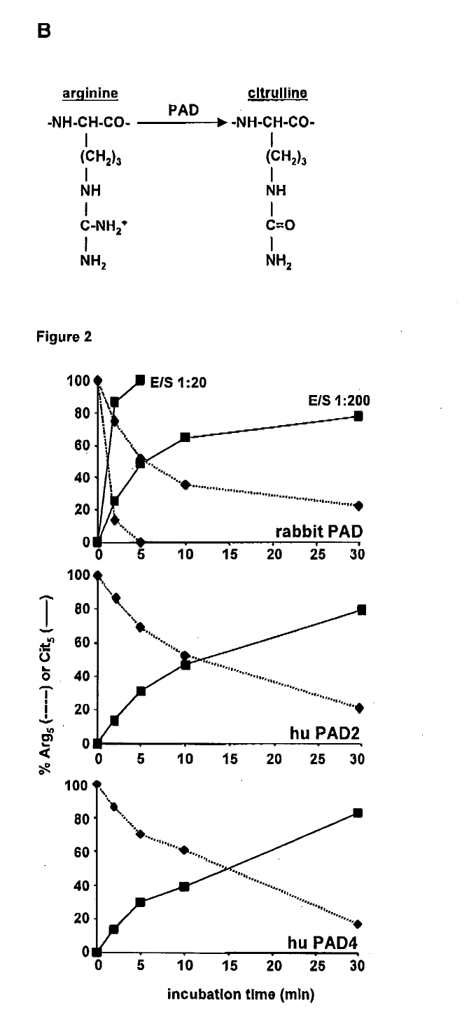

The presence or absence of a citrulline in natural CXCL8 was evidenced by a combination of mass spectrometry and capillary protein sequencing. Incubation of CXCL8 with rabbit PAD rapidly converted recombinant CXCL8(1-77) into CXCL8(1-77)Cit₅ indicating that this enzyme is responsible for the posttranslational modification of natural CXCL8 (FIG. 2A). Deimination of an Arg to Cit, which results in only a single mass unit increase of the Mᵣ, is a natural posttranslational modification that has previously been observed for proteins such as vimentin, keratin, filaggrin, α-enolase, myelin basic protein (MBP), the protease inhibitor antithrombin and some nuclear proteins, e.g. histons, nucleophosmin and p300 but was never reported before for cytokines or chemokines. To date, five human genes have been described that encode for PAD, i.e. PADI1,2,3,4 and 6. Modification of Arg to Cit has been linked to autoimmune diseases such as rheumatoid arthritis (RA) and multiple sclerosis (MS) (Girbal-Neuhauser E. et al., 1999, J. Immunol. 162:585-594). Citrullination of proteins is even suggested to initiate the generation of autoimmune reactions. In serum of rheumatoid arthritis (RA) patients, citrullinated fibrin appeared to be the major target of the anti citrulline peptide antibodies. A synthetic cyclic citrullinated peptide (CCP) construct is used nowadays as a diagnostic tool to distinguish RA from other arthritic disorders and possesses a highly predictive value for future development of RA in healthy individuals and patients with undifferentiated arthritis (Nielen M. M. et al., 2004, Arthritis Rheum. 50:380-386). In addition to citrullination of structural proteins, increased levels of citrullinated antithrombin were detected in the plasma of RA patients. It was reported that in a Japanese population, SNPs in the human PAD/4 gene were strongly associated with susceptibility for RA, although this association was not found in studies that were conducted in the UK and France. The

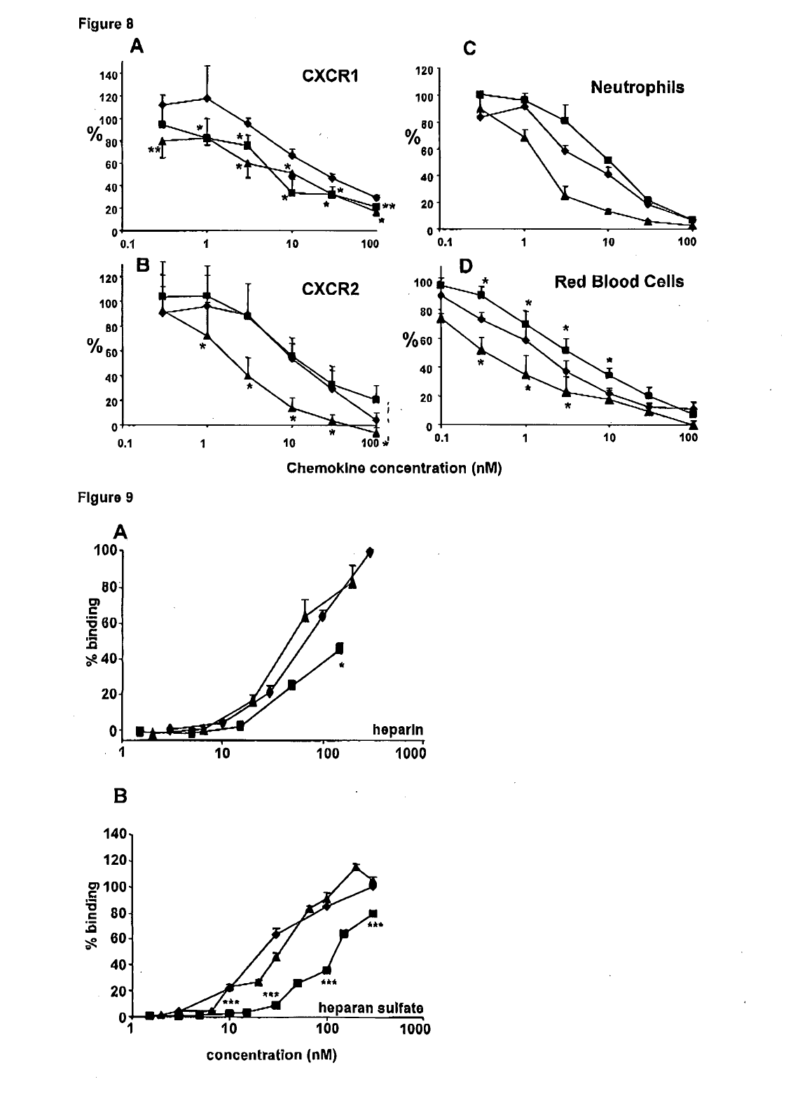

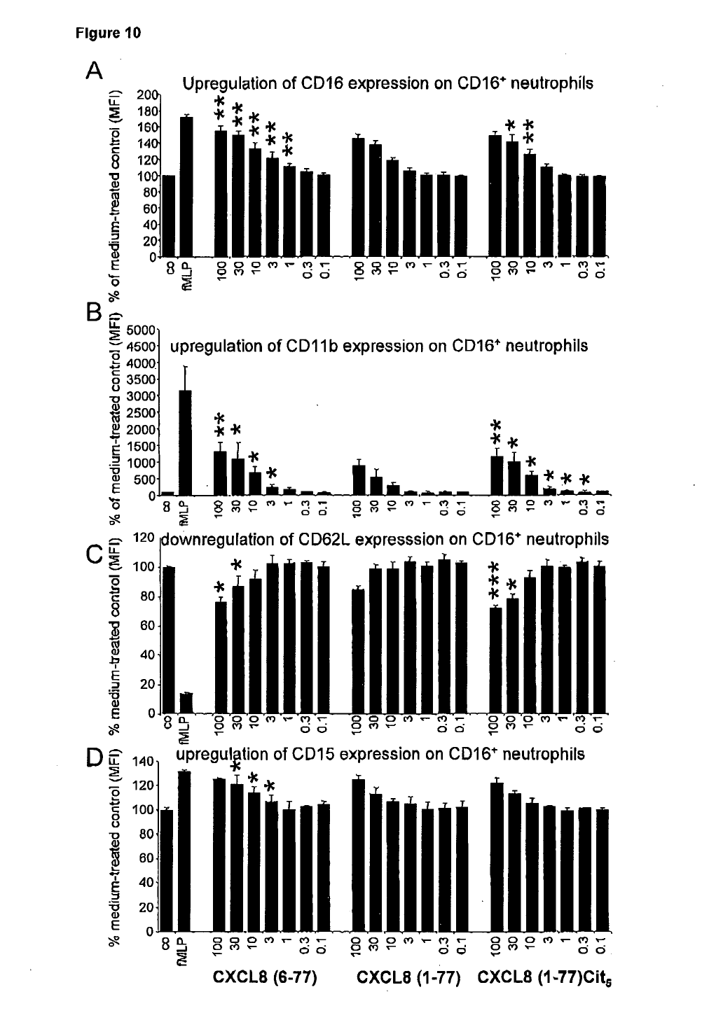

PAD used in this study was equivalent to human PAD4 which is expressed in leukocytes. Deimination of Arg was also reported in association with MS. PAD2-dependent citrullination of MBP was suggested to play an important role in MS patients. Citrullination of MBP is increased in MS patients and exposes immunodominant epitopes, it renders MBP more susceptible to cleavage with cathepsin D and may therefore initiate loss of myelin stability. Moreover, here we report that also both human PAD2 and human PAD4 efficiently modified Argₛ in CXCL8 with comparable potency to rabbit PAD (FIG. 2B). This invention for the first time reports natural site-specific citrullination of cytokines and chemokines, e.g. the chemokine IL-8/CXCL8. In contrast to most other previously identified substrates, which were primarily structural proteins, CXCL8 directly affects immune functions and angiogenesis. Therefore, PAD-activity may not only promote auto-immunity on long term through the production of anti-citrulline antibodies but PAD may also directly interfere in immunological processes. Since cytokines such as IL-1β and tumor necrosis factor (TNF)-α are important players in autoimmune diseases such as rheumatoid arthritis, the possible interaction of PAD with IL-1β and TNF-αwas investigated. PAD failed to convert Arg to Cit in IL-11 but was able to modify both NH₂-terminal Arg to Cit in TNF-α (see example 4) (with comparably positioned NH₂-terminal Arg as in CXCL8) shows that citrullination is a cytokine-specific phenomenon. The rapid in vitro citrullination of the Argₛ in CXCL8 by PAD under conditions that do not disrupt the three-dimensional chemokine structure and the isolation of PBMC-derived citrullinated CXCL8(1-77)Cit₆ underscore the efficiency of the enzymatic conversion of this chemokine by PAD. Actually, most of the in vitro incubations with PAD on other reported substrates were performed in the presence of reducing agents which may render Arg residues, which are protected by the three dimensional structure of the protein, accessible for enzymatic processing. In vitro, CXCL8(1-77)Cit₆ and uncitrullinated CXCL8(1-77) were equally potent in neutrophil chemotaxis assays although a significantly reduced CXCR2 dependent signaling efficiency was detected on receptor transfected cells (FIGS. 4, 5 and 6). However, in contrast to CXCL8(1-77), CXCL8(1-77)Cit₅ was completely resistant to thrombin-cleavage into CXCL8(6-77) (FIG. 7). In addition to the loss of the proteolytic cleavage site in CXCL8, CXCL8(1-77)Cit₈ competes more efficiently compared to CXCL8(1-77) for binding of ¹²⁵I-CXCL8 to cells transfected with CXCR1 (FIG. 8A). In contrast, both molecules bound equally potent to CXCR2 (FIG. 8B) and neutrophils (FIG. 8C). CXCL8, in addition to a number of other inflammatory chemokines also binds to DARC, a receptor that is expressed on red blood cells and endothelial cells for which so far no signalling properties have been described. No significant difference in binding to red blood cells was detected between CXCL8 (1-77) and CXCL8(1-77)Cit₅ (FIG. 8D). In addition to seven transmembrane-spanning receptors, chemokines also bind to glycosaminoglycans. Citrullination of the first Arg in CXCL8 reduced the GAG-binding properties (to both heparin and heparan sulphate) of this chemokine (FIG. 9). Chemokines are known to alter adhesion molecule expression through receptor-dependent signal transduction mechanisms. Treatment of neutrophils in total blood with CXCL8 (1-77), CXCL8(6-77) or CXCL8(1-77)Cit₅ resulted in reduced L-selectin (CD62L) and enhanced sialyl Lewis X (CD15) and integrin (CD11b) expression (FIGS. 10B, C and D). In addition also the expression of F_{c} receptor IIIB (CD16) augmented (FIG. 10A). Truncated CXCL8(6-77) was the most efficient upregulator of CD11b, CD15 and CD16 and downregulator of L-selectin. Compared to CXCL8(1-77), CXCL8(1-77)Cit₅ treatment of neutrophils resulted in a more potent increase in CD16 and CD11b expression and in a more pronounced shedding of L-selectin (FIG. 10 A, B and C). However, no significant difference in increase of CD15 expression was detected with CXCL8(1-77) and CXCL8(1-77)Cit₅. Thus, citrullination of CXCL8 is likely to result in decreased rolling (due to decreased L-selectin expression) and increased attachment (due to enhanced integrin expression levels) of neutrophils to endothelial layers.

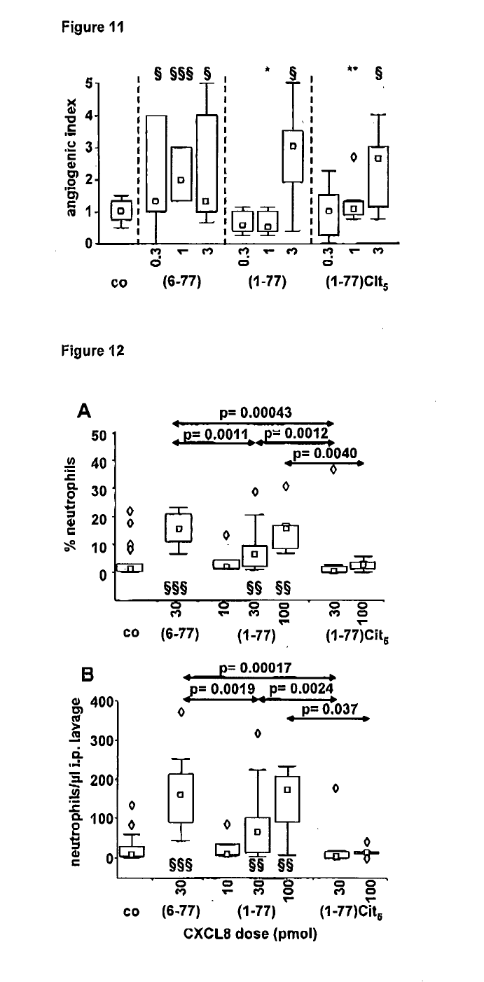

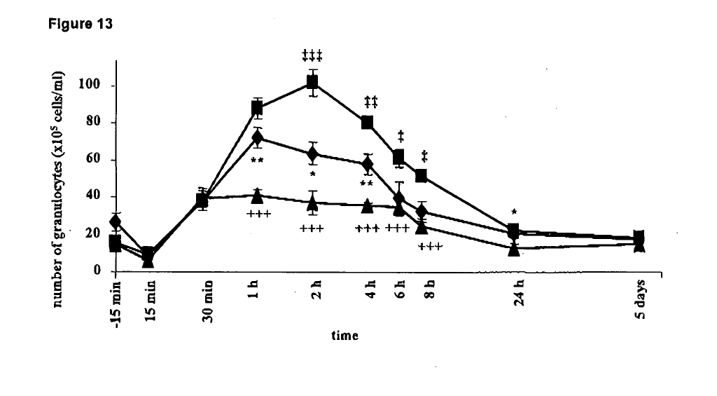

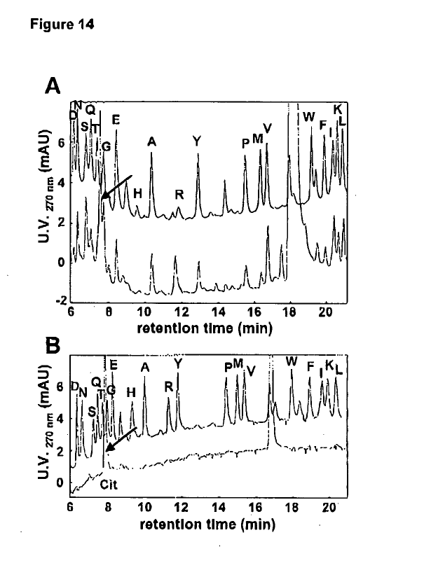

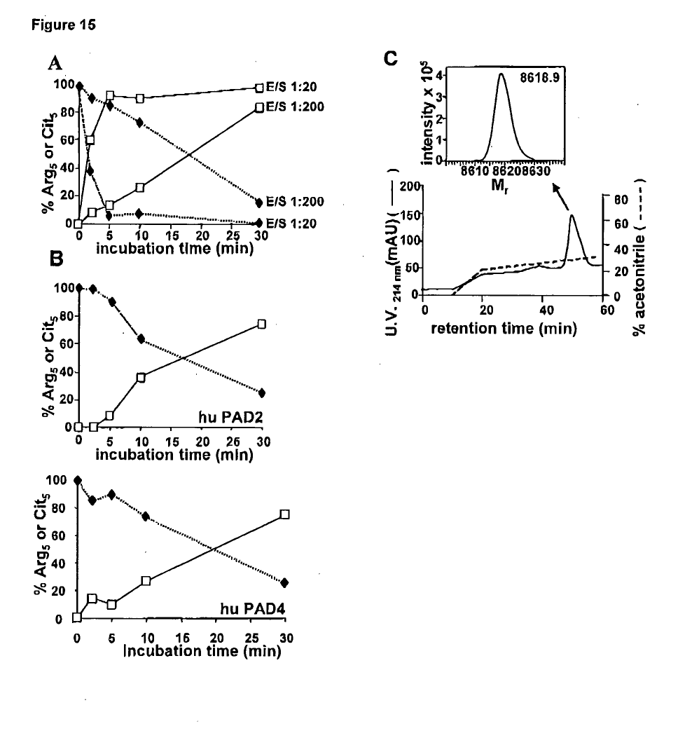

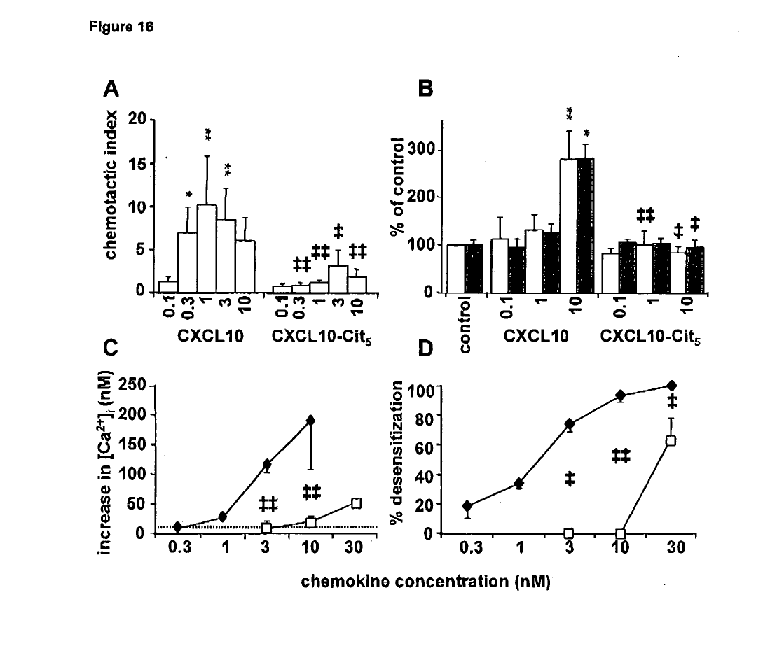

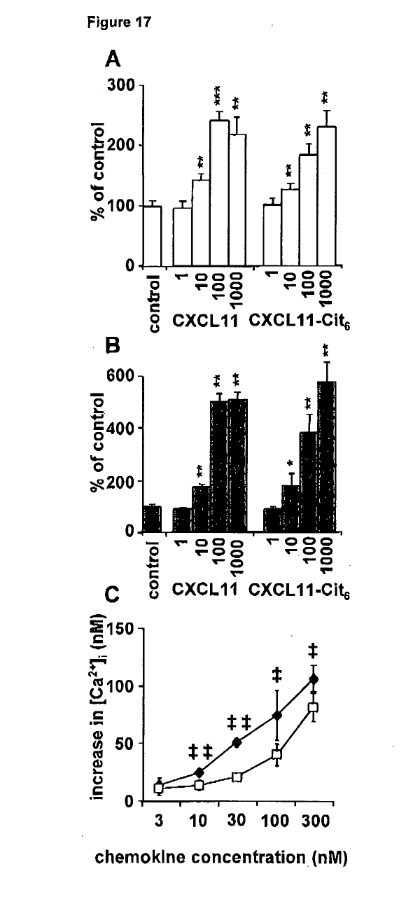

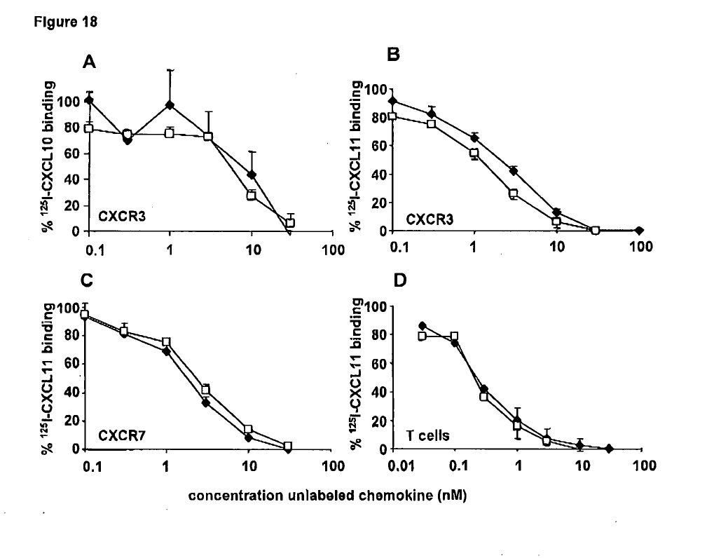

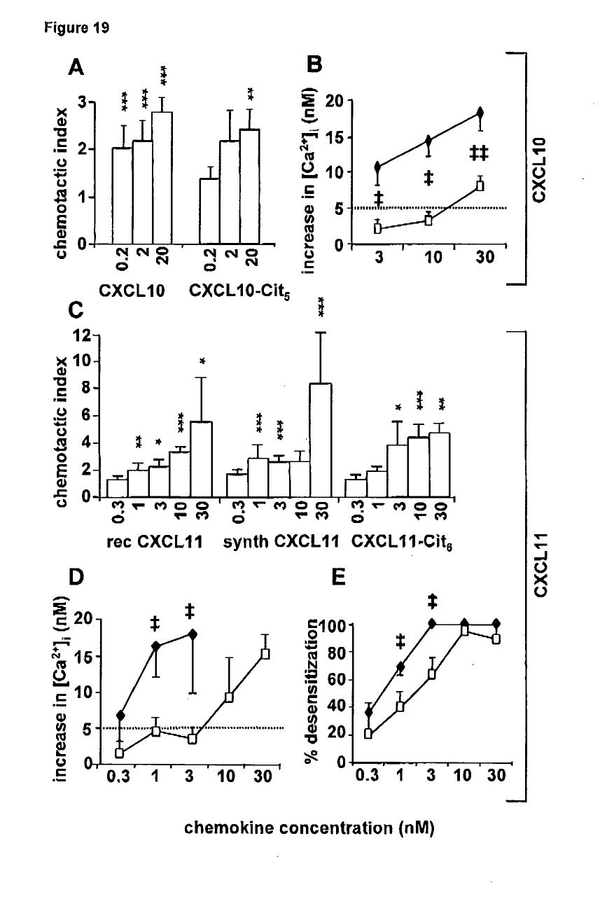

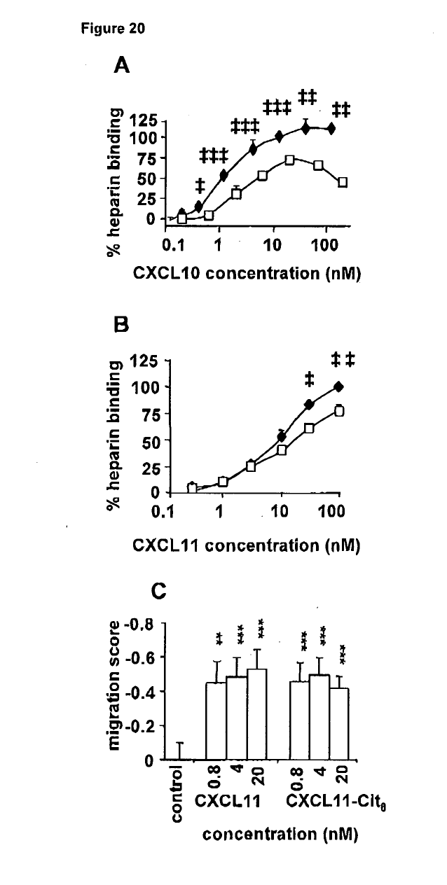

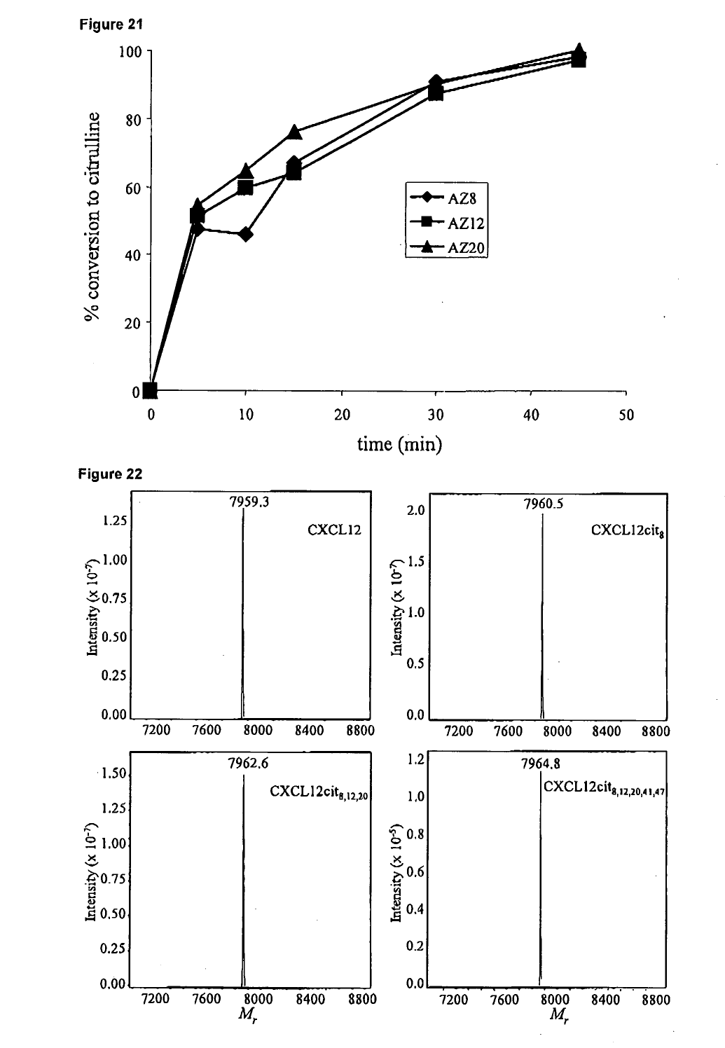

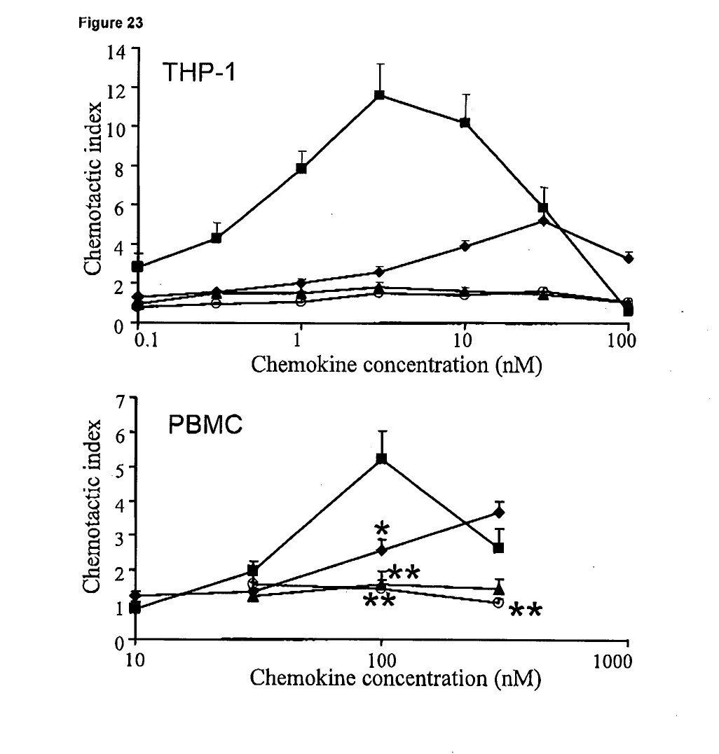

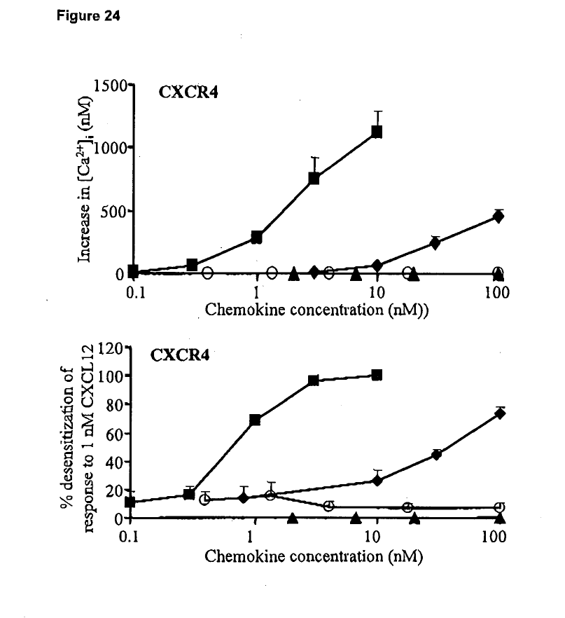

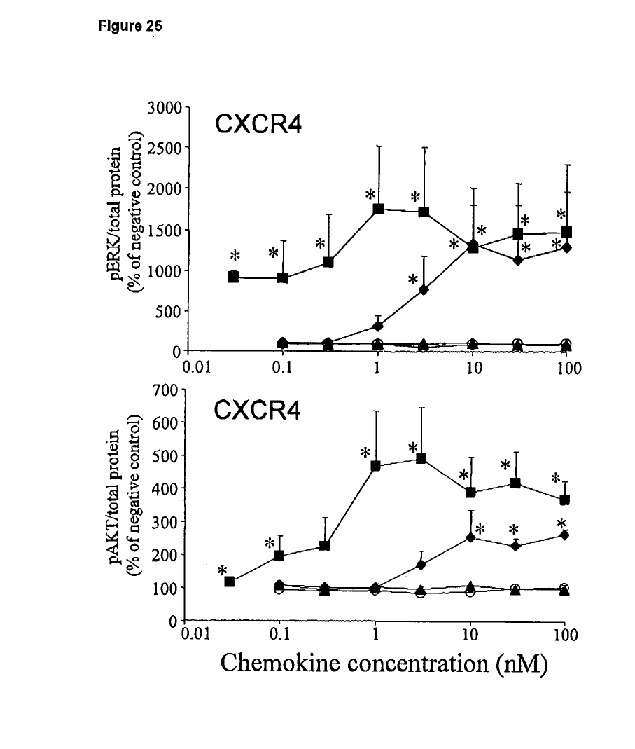

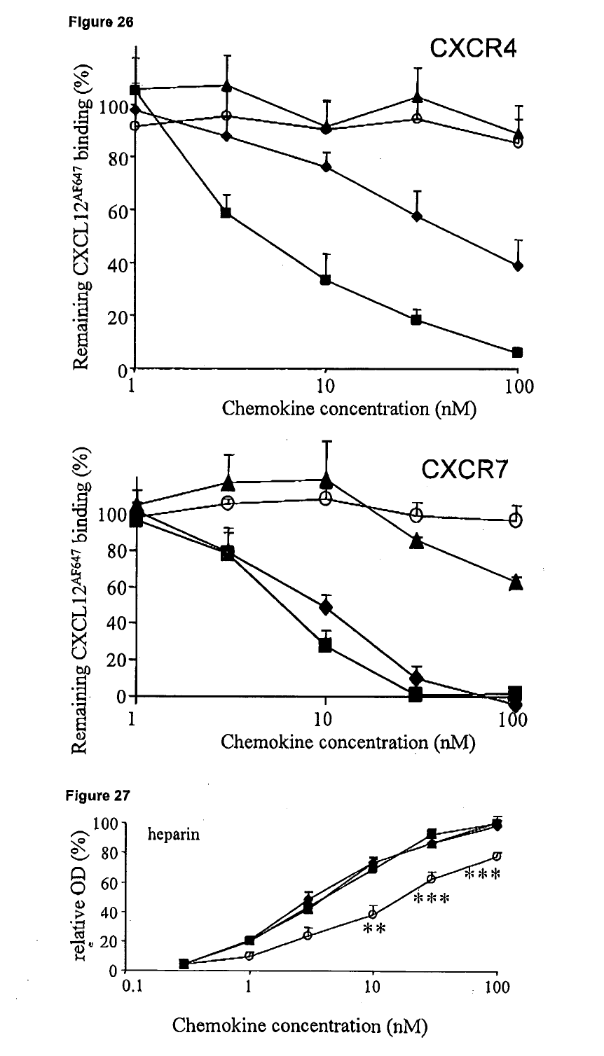

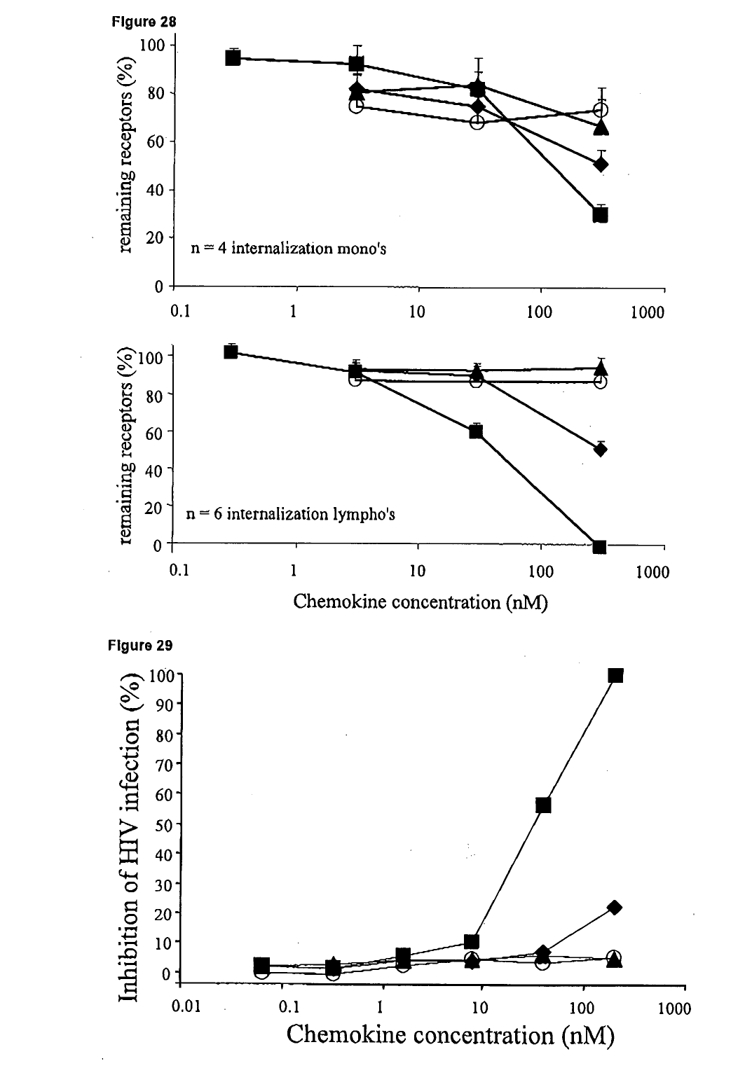

Because conversion of CXCL8(1-77) into CXCL8 (6-77) rapidly occurs in the presence of leukocytes and since this proteolytic NH₂-terminal truncation results in enhanced in vitro activity of CXCL8, the increased stability of CXCL8(1-77)Cit₅ might affect the in vivo activity of this chemokine. Alternatively, the reduced GAG-binding and CXCR2-signaling activity might result in reduced in vivo activity. CXCL8(1-77)Cit₅, unlike CXCL8(1-77), was devoid of chemotactic activity when up to 100 pmol was injected i.p. (FIG. 12). In contrast, at this amount truncated CXCL8 (6-77) had increased in vivo chemotactic activity compared to intact CXCL8(1-77) confirming the previously reported in vitro observations. Thus, citrullination of a single Arg in CXCL8 hampers local neutrophil driven inflammation despite the retained in vitro chemotactic activity. In contrast to i.p. injection, i.v. administration of CXCL8(1-77) provoked a more pronounced granulocytosis compared to CXCL8(6-77) and citrullination of CXCL8(1-77) resulted in still higher numbers of circulating granulocytes at 1 h to 4 h post injection (FIG. 13). The enhanced mobilization of granulocytes upon systemic application of CXCL8Cit₅ opens new perspectives in view of cytokine and chemokine usage for leukocyte mobilization to the blood circulation.

In addition to its function as a neutrophil chemotactic and activating protein, CXCL8 has also angiogenic activity. In accordance with the in vitro chemotaxis assays on neutrophils, CXCL8(6-77) was more potent compared to intact CXCL8 as an angiogenesis inducing agent in the cornea assay in rabbits (FIG. 11). However, no difference in angiogenic activity could be detected between CXCL8(1-77) and CXCL8(1-77)Cit₅. These findings indicate that the angiogenic activity of CXCL8 is primarily not dependent on CXCR2-mediated and GAG-mediated pathways or uses different signal transduction mechanisms.

The interaction between chemokines and proteases is a key process in the regulation of the immune response. Chemokines not only trigger the release of proteases but also act as substrates. Several natural posttranslational modifications have been reported to affect the biological nature of the chemokine substrate, e.g. the conversion of CXCL8 (1-77) into CXCL8 (6-77) by plasmin or thrombin potentiates its biological activity, facilitating a positive feedback loop. Moreover, posttranslational modifications seem to complicate the pattern of apparent redundancy in chemokine-receptor interactions. For example, after DPPIV/CD26 processing the CC chemokine CCL5 discloses an enhanced receptor interaction with CCR5, parallel though with a decreased affinity for CCR1 and CCR3. This NH₂-terminal truncation of CCL5 does not reduce lymphocyte attraction, but results in a loss for monocyte chemotaxis. Through posttranslational modifications, proteases thus influence the selectivity of the attracted leukocyte subset during inflammation or infection. In addition, various tumor cells constitutively express chemokines e.g. granulocyte chemotactic protein-2 (GCP-2/CXCL6), provoking leukocyte infiltration from blood vessels into the tumor tissue which is hypothesized to leaving a pathway of degraded extracellular matrix open for metastasis. On the other hand, proteases can also influence other properties of chemokines, e.g. the DPPIV/CD26-processed isoform of CCL5 was more potent at protecting CD4⁺ cells against HIV-1 infection, due to the increase in affinity for CCR5. NH₂-terminal cleavage of CXCL10 by DPPIV/CD26 into CXCL10 (3-73) impairs its CXCR3 signaling and chemoattractive capacity, however, does not interfere with its antiangiogenic character. In contrast, the subsequent cleavage of CXCL11 by DPPIV/CD26 and aminopeptidase N (APN)/CD13 not only weakens its receptor binding, signaling and chemoattractive characteristics, it also reduces its angiostatic nature.

In this invention, we describe a non proteolytic though enzymatic posttranslational modification of chemokines with profound effect on the biological activity. Indeed, we purified a novel natural CXCL10 isoform from stimulated pooled PBMCs, i.e. citrullinated CXCL10. We confirmed the conversion of arginine at position 5 into citrulline to be exerted by PAD but with the retention of arginine at position 8. In view of the facts that PAD is co-expressed with citrullinated peptides in the synovium of RA and that PAD is expressed in inflamed tissue, it is likely that CXCL10-Cit₅ also occurs in vivo, since protein levels of CXCL10 were enhanced in synovial fluid samples of patients suffering from RA and spondylarthropathies. This novel posttranslational modification of a positively charged hydrophilic amino acid into a neutral amino acid revealed to be of great importance, since CHO-CXCR3 cells did not respond to CXCL10-Cit₅ in a standard chemotaxis assay as well as in signaling experiments, though CXCR3 binding properties were preserved. In addition, PAD was also shown to deiminate CXCL11. However, this conversion had no impact on its CXCR3 binding or signaling capacity nor in CXCR7 binding competition. These results point to a restricted regulation by PAD of CXCR3-mediated immune responses, thus to a CXCL10-specific modulation. On top, these data demonstrate that structurally distinct regions of CXCL10 are responsible for different functions as previously reported in structure-function studies through NH₂-terminal truncation and alanine mutagenesis experiments. Further investigation in a more physiological context showed an interesting cell-type dependent response towards citrullinated CXCL10 and CXCL11. In T cells, a decrease in biological activity was observed in chemotaxis and calcium signaling assays in response to CXCL10-Cit₅. Though in contrast to CHO-CXCR3 cells, where CXCL10-Cit₅ was unable to induce chemotaxis, CXCL10-Cit₅ remained slightly active in T cells. In addition, CXCL11-Cit₆ was also tenfold less potent at inducing calcium signaling, but not migration in lymphocytes. To explore this cell-specific behavior, CXCL11 and its deiminated isoform were tested in an in vitro wound healing assay. CXCL11 and CXCL11-Cit₆ turned out to be equally potent at inhibiting microvascular endothelial cell migration, in agreement with previous observations for CXCL10(3-73) which lacks CXCR3 signaling and chemotactic activity, whilst retaining angiostatic activity.

Overall, the effects of citrullination Observed in this manuscript, reveal a new dimension in the fine regulation of chemokine activity in the immune response. For CXCL10, this posttranslational modification by PAD causes a negative feedback loop, possibly implicated in protective processes in inflamed tissue by decreased lymphocyte recruitment. Since our results show both a cell-type and chemokine dependent response on citrullinated CXCL10 and CXCL11, it is likely that the effects of PAD reside rather local.

Chemokines play a relevant role in a number of pathologic conditions, mainly based on their overexpression in disorders associated with leukocyte infiltration. CXCL12 is a pluripotent chemokine which, unlike most proteins of the chemokine family, is well conserved in almost all animal species and activates monogamously its functional receptor CXCR4. Both the ligand and its receptor were originally found to be essential for B lymphopoiesis and bone marrow myelopoiesis. However, the SDF-1/CXCR4 signaling pathway is critically involved in development and various pathophysiological phenomena, including hematopoiesis, angiogenesis, atherosclerosis, cancer growth, metastasis, and human immunodeficiency virus infection. Consistently, mice lacking either the SDF-1 or its receptor exhibit defects that extend beyond the immune system, revealing roles for this chemokine in the genesis of the circulatory and central nervous systems. The chemokine system is not only essential for leukocyte homing during physiological and pathological conditions but is also a target for viral mimicry. In order to infect human leukocytes, HIV-1 has to bind to CD4 and a co-receptor that belongs to the G-protein coupled receptors. The majority of HIV-1 strains use either CCR5 (R5-tropic strains), CXCR4 (X4-tropic strains) or both CCR5 and CXCR4 (dual-tropic strains). Therefore, these co-receptors are novel targets for viral therapy and novel classes of anti-HIV drugs, i.e. the entry inhibitors.

In particular, CXCL12 protein is overexpressed in rheumatoid synovium compared with osteoarthritic synovial membrane. In RA, CXCR4 and CXCL12 were reported to be important factors for T and B lymphocyte migration. In this invention, we describe that during autoimmune disease SDF-1 and PAD, which can enzymatically modify this chemokine, are co-expressed.

CXCL12 and PAD are known to play a role in various autoimmune diseases and limited posttranslational modification of chemokines such as CXCL12 may profoundly affect the receptor binding, signalling and chemotactic properties of these proteins. In addition, CXCR4 specific antagonists, e.g. AMD3100 prevents joint inflammation in collagen-induced arthritis. PAD generates auto-antigens in RA patients that lead to the generation of anti-citrulline antibodies. These antibodies are more potent diagnostic tools than rheumatoid factor and can be detected in RA patients before the appearance of clinical symptoms (Nielen M. M. et al., 2004, Arthritis Rheum. 50:380-386). In multiple sclerosis, citrullination of myelin basic protein is enhanced and CXCL12 was originally reported as a stromal cell-derived factor in the central nervous system. In this invention, we observed intestinal co-expression of CXCL12 and PAD in patients suffering from Crohn's disease. Convergently, treatment of CXCL12 with PAD resulted in rapid deimination (citrullination) of several Arg. Chemically synthetized citrullinated CXCL12 isoforms with variable degree of citrullination were used to study the effect of Arg deimination on the receptor binding properties and biological activities of CXCL12. Even deimination of only the first Arg in the CXCL12 sequence strongly reduced the CXCR4 binding potency of this chemokine. In addition, CXCL12-Cit₈ showed significantly reduced calcium signalling, as well as ERK and Akt/PKB phosphorylation. Further citrullination of CXCL12 on three (CXCL12-Cit_{8,12,20}) or five (CXCL12-Cit -8,12,20,41,47) Arg resulted in complete loss of CXCR4 binding and signalling properties. Finally, this led to reduced (for CXCL12-Cit₈) or completely impaired (in case of CXCL12-Cit_{8,12,20} and CXCL12-Cit_{8,12,20,41,47}) leukocyte chemotactic activity.

In contrast to the profound effect of the presence of a single Cit on the interaction of CXCL12 with CXCR4, CXCL12-Cit₈ retained its binding properties on CXCR7. This indicates that different residues in the CXCL12 sequence are crucial for either CXCR4 or CXCR7 binding. The natural CXCR4 ligand CXCL12 inhibited HIV-infection with X4-tropic strains through competition for binding to CXCR4. In accordance with the binding and signalling data on CXCR4, citrullination of CXCL12 reduced its antiviral potency on lymphocytic cells.

The present invention will now be illustrated by means of the following examples which are provided without any limiting intention.

### EXAMPLES

### Example 1

### Natural Posttranslational Modification of CXCL8 by Peptidylarginine Deiminase

A. Materials and Methods

Reagents and Cell Lines

Recombinant human IL-1β, IFN-γ , CXCL8(1-77) and truncated CXCL8(6-77) were obtained from PeproTech (Rocky Hill, N.J., USA). Human plasma derived thrombin (2532 NIH units/mg) and plasmin (3-6 units/mg), PAD purified from rabbit skeletal muscle (200 units/mg) and double stranded (ds)RNA polyriboinosinic:polyribocytidylic acid (polyrl:rC) were purchased from Sigma-Aldrich (St. Louis, Mo., USA). Lipopolysaccharide (LPS from E. coli 0111:64) was from Difco Laboratories (Detroit, Mich., USA). Human embryonic kidney (HEK) 293 cells transfected with CXCR1 or CXCR2 were kindly provided by Dr. J. M. Wang (NCI-NIH, Frederick, Md., USA) and were cultured in Dulbecco's Modified Eagle's Medium with 4.5 g/l glucose (DMEM; Cambrex BioScience, Vervlers, Belgium) supplemented with 10% FBS (fetal bovine serum) and 800 μg/ml geneticin (Gibco, Paisley, Scotland). Endotoxin concentrations were evaluated with the Limulus amoebocyte lysate test.

### Leukocyte Isolation and Production of Natural Chemokines

Leukocytes were isolated from fresh human buffy coats obtained from the blood transfusion center of Leuven as previously described (Proost P. et al. J. Immunol., 1993, 150:1000-1010). Briefly, erythrocytes were removed by sedimentation in hydroxyethyl-starch (Plasmasteril, Fresenius, Bad Homburg, Germany). Mononuclear cells and granulocytes were separated by gradient centrifugation on Ficoll-sodium metrizoate (Lymphoprep, Nycomed, Oslo, Norway). Remaining erythrocytes were removed from the granulocyte pellets by hypotonic shock. Granulocytes from individual donors were used for chemotaxis and calcium signaling experiments. Purified peripheral blood mononuclear cells

(PBMC) from individual donors were cultured in RPMI 1640 enriched with 10% FBS and 0.05% (w/v) gentamycin (Invitrogen, Carlsbad, Calif., USA), activated with 2 μpg/ml phytohaemagglutinin (Sigma-Aldrich) for 2 to 5 days and then washed with medium and stimulated with 50 U/ml human recombinant IL-2 (PeproTech) every 2 or 3 days. Lymphoblast chemotaxis, calcium signaling and receptor binding experiments were performed two weeks after PHA-activation and two days after IL-2 stimulation. Alternatively, for chemokine production, PBMC from 24 buffy coats were pooled and induced at 5×10⁶ cells/ml with 10 μg/ml of the polyrl:rC and 20 ng/ml IFN-γ in RPMI 1640 (Cambrex BioScience) containing 2% FBS. The conditioned media were stored at −20° C. until purification.

### Purification and Identification of Natural Chemokines

Natural human chemokines were purified to homogeneity using a four-step purification procedure (Proost P. et al. J. Immunol., 1993, 150:1000-1010). The CXCL8 concentration in column fractions was determined with a specific sandwich ELISA as previously described (Proost P. et al. Arthritis Res. Ther., 2006, 8:R107). Proteins were eluted from the final reversed phase (RP-)HPLC column (2.1×220 mm Brownlee C8 Aquapore RP-300 column, PerkinElmer) in an acetonitrile gradient in 0.1% trifluoroacetic acid (TFA) and U.V. absorption was monitored at 214 nm. A minor portion of the column flow (1/150) was split on-line to an electrospray ion trap mass spectrometer (Esquire L C, Bruker, Bremen, Germany). Profile MS spectra were collected every 0.1 second and averaged profile spectra were calculated over the U.V. adsorption peaks that contained CXCL8 immunoreactivity. The measured average relative molecular mass (Mᵣ) was compared with the theoretical average M, of CXCL8. The NH₂-terminal sequence of the isolated chemokines was determined by Edman degradation on a 491 Procise cLC protein sequencer (Applied Biosystems, Foster City, Calif., USA).

In vitro Citrullination or Truncation

Proteins were incubated with PAD in 40 mM Tris-HCl, pH 7.6, 2 mM CaCl₂ at 37° C. Deimination was stopped by adding 0.1% TFA and samples were either desalted on C18 ZipTip (Millipore, Billerica, Mass., USA) prior to mass spectrometry or spotted on PVDF membranes (ProSorb; Applied Biosystems) prior to Edman degradation. For use in bioassays, citrullinated proteins were purified by RP-HPLC on a C-8 Aquapore RP-300 column (1×50 mm) and 2% of the flow was converted to the on-line mass spectrometer.

Alternatively, recombinant CXCL8 was incubated at a 1:100 enzyme/substrate molar ratio with human thrombin (20 U/ml; Sigma-Aldrich) in PBS containing 1 mM CaCl₂ or with plasmin in Tris pH 7.4 at 37° C. for different time periods. CXCL8 proteolysis was stopped by either adding 0.1% TFA prior to protein sequence analysis on PVDF membranes or by adding 50 mM Tris-HCl, pH 6.8, 4% SDS, 12% glycerol, 2% 2-mercaptoethanol and 0.019% Brilliant blue G followed by heating for 5 min at 95° C. prior to SDS-PAGE.

In vitro Chemotaxis and Signaling Assays

Neutrophil chemotaxis was performed in Boyden microchambers (Neuro Probe, Cabin John, MD, USA) as previously described (Proost P. et al. J. Immunol., 1993, 150:1000-1010). The cells were counted microscopically at 500× magnification in 10 oil immersion fields. The chemotactic index was calculated as the number of cells migrated to the sample divided by the number of cells that spontaneously migrated to the sample dilution medium (HBSS+0.1% HSA).

Changes in intracellular calcium concentration ([Ca²1]ᵢ) were measured by fluorescence spectrometry on an LS50B spectrofluorimeter (PerkinElmer) with the ratiometric fluorescent dye Fura-2/AM (Molecular Probes Europe BV, Leiden, Netherlands).

Additional signaling tests were performed to evidence in vitro cell activation. The extracellular signal-regulated kinase 1 and 2 (ERK1/2) and protein kinase B (PKB)/Akt assays were performed as previously described (Proost P. et al. Arthritis Res. Ther., 2006, 6:R107). Briefly, HEK-293 cells transfected with CXCR2, were cultured overnight in serum-free medium, Subsequently, the cells were stimulated with test samples in medium enriched with 0.5% FCS. The signal transduction was stopped by cooling the plates on ice and adding ice-cold PBS. The cells were washed with cold PBS and cell lysis was induced in PBS containing 1 mM EDTA, 0.5% Triton X-100, 5 mM NaF, 6 M urea, protease inhibitor cocktail for mammalian tissues and phosphatase inhibitor cocktail 1 and 2 (Sigma-Aldrich). After 10 min, the lysate, collected by scraping off the cells, was incubated on ice for 45 min and centrifuged. The protein concentration in the supernatant was examined by the bicinchoninic acid (BCA) protein assay (Pierce, Rockford, Ill., USA) and the amount of ERK1/2 and PKB/Akt phosphorylation was determined using specific ELISAs for phospho-ERK1 and 2 (pg phospho-ERK1/2 per mg of total protein) and for phospho-Akt (R&D Systems).

### Glycosaminoglycan (GAG) Binding Assay

GAG binding was evaluated by immobilizing low molecular weight heparin or heparin sulphate (Sigma-Aldrich) on EpranEx plates (Plasso Technology Ltd., Sheffield, UK). Briefly, 25 μg/ml of heparin diluted in phosphate-buffered saline (PBS) was coated overnight at room temperature on 96-well plates. Plates were washed three times with standard assay buffer (100 mM NaCl, 50 mM NaAc, 0.2% (v/v) Tween-20 pH 7.2) and blocked at 37° C. with standard assay buffer enriched with 0.2% (w/v) gelatin for heparin binding or with 0.2% bovine serum albumin for heparin sulphate binding. The captured chemokines on GAG treated plates were detected with biotinylated anti human CXCL8 (PeproTech Inc.) and consequently by peroxidase conjugated streptavidin (Jackson ImmunoResearch Laboratories, West Grove, Pa.). Peroxidase activity was quantified by measuring the conversion of 3,3′,5,5′-tetramethylbenzidine (TMB; Sigma) at 450 nm. No difference in specificity of the biotinylated or monoclonal antibody for intact CXCL8 or its isoforms was uncovered.

### Adhesion Molecule Staining in Whole Blood

Blood samples from healthy volunteers were collected by 21 G needle venepuncture in a lithium heparin-treated tube (vacutainer, BD Biosciences, San Jose, Calif.). Blood samples were diluted in warm PBS (37° C.) to 10⁸ leukocytes/ml in sterile tubes containing prewarmed dilutions of chemokines or fMLP, After incubation for 10 minutes at 37° C. in the presence of 5% CO₂ the samples were immediately placed on ice and ice-cold PBS was added to the tubes to stop the signal transduction cascades. Consequently, the Fc-receptors were blocked by storing the samples in ice-cold PBS containing 2% FCS for 15 minutes, prior to FAGS-staining. Antibodies used for staining were purchased at BD Biosciences (anti human CD16 PE, CD11b CyChrome, isotype IgG1 κ-type FITC) or at eBioscience (San Diego, Calif.) (CD15 FITC, CD62L APC, isotype control IgM FITC). Subsequently, red blood cell lysis was performed after washing the cells three times with PBS+2% FCS and 15 minutes of fixation with PBS+2%FCS+2% formaldehyde.

### Angiogenesis and Leukocytosis Assays

The in vivo angiogenic, activity of chemokines was tested in the rabbit cornea micropocket model. Different amounts of chemokine in 1 μl Dulbecco's PBS (Cambrex) were allowed to dry on 5 μl Hydron pellets and implanted 1 mm from the limbus into corneal micropockets of anesthetized New Zealand white rabbits. Neovascularization of the cornea was scored daily from day 4 till day 8 after implantation of the pellet. Maximal neovascularization obtained between days 5 and 7 was used for comparison. induction of leukocytosis was measured in New Zealand white rabbits (±3 kg) by i.v. injection (1 ml) of 10 μg of chemokine in PBS. Blood samples were collected by bleeding at a peripheral ear vein in potassium EDTA coated tubes at several time intervals before and after treatment. Total leukocytes were counted in triplicate and the percentage of granulocytes was determined in quadruplicate by differential 100-cell counts on blood smears stained with Hemacolor solutions.

### Intraperitoneal Infiltration of Granulocytes

Granulocyte mobilization into the peritoneal cavity was determined, in NMRI female mice (Elevage Janvier, Le Genest Saint Isle, France) by i.p. injection (200 μl in saline) of pyrogen-free CXCL8(1-77), CXCL8(6-77), citrullinated CXCL8 or saline (0.9% NaCl) in an aseptic environment. After 2 or 4 h, the mice were sacrificed and the intraperitoneal cavity was washed with 5 ml of saline enriched with 2% FBS and 20 U/ml heparin. The total amount of leukocytes in the intraperitoneal lavage was determined in duplicate. Cytospins were stained with Hemacolor solutions (Merck) for evaluation of the percentage of granulocytes by differential 100-cell counts in triplicate.

### B. Results

### Identification of Naturally Citrullinated Isoforms of CXCL8

Natural CXCL8 was produced by stimulated PBMC and purified by adsorption to controlled pore glass, subsequent heparin affinity chromatography, cation exchange chromatography and C8 RP-HPLC as previously described (van Damme, J. et al., 1988. J. Exp. Med. 167:1364-1376). CXCL8 isoforms were detected in the column fractions by specific ELISA and identified by both mass spectrometry and amino acid sequencing using Edman degradation. Although most of the PBMC-derived CXCL8 was truncated by 5 or more NH₂-terminal amino acids resulting in the NH2-terminal sequence SAKELRXQXIK . . . , many fractions contained intact CXCL8, i.e. CXCL8(1-77) (sequence AVLPRSAKELRXQXIK . . . ). Due to their instability in the Edman degradation, the phenyl thiohydantoin (PTH)-Cys residues on positions 12 and 14 of intact CXCL8 were not detected (indicated as X) by Edman degradation and subsequent RP-HPLC (Table I). Remarkably, in some fractions the PTH-Arg at position 5 (PTH-Arg_{y}) in the sequence of natural CXCL8 was also not identified (indicated as B), whereas the PTH-Arg₁₁ present in the ELR-motif was always detected (sequence AVLPBSAKELRXQXIK . . . ). In contrast, recombinant CXCL8 clearly showed the presence of this PTH-Arg₅, whereas the PTH-Cys remained undetectable upon sequence analysis. Instead of a PTH-Argₛ peak, in natural CXCL8(1-77) an unidentified compound eluted from the RP-HPLC column of the protein sequencer in between PTH-Thr and PTH-Gly (FIG. 1A). On classical, non-capillary ⁻protein sequencers with an HPLC column with a diameter of 2.1 mm instead of 0.8 mm, the unidentified residue could not be detected since it co-eluted with the only partially separated PTH-Thr and PTH-Gly peaks. The high background on PTH-Thr and PTH-Gly and the weak signal for PTH-Arg on these non-capillary protein sequencers also explain why this modification was not reported before. In addition, the experimentally determined Mᵣ of 8917.1 to 8919 (Table I) of natural CXCL8(1-77) with the observed modification on position 5 did not significantly differ from the theoretical Mᵣ (8918.44) of CXCL8(1-77). Based on these mass spectrometry and Edman degradation data, the difference between natural and recombinant CXCL8 could not be explained by a mutation of Arg to one of the other 19 classical amino acids. Therefore, amino acids generated by posttranslational modification of Arg were considered. The only known side chain alteration on Arg which results in a minimal change in M, (one mass unit) and which was reported to naturally occur in some other structural proteins is the modification of Arg into citrulline (Cit) (FIG. 1B). Therefore, L-citrulline was loaded on the filter of the protein sequencer and after one Edman degradation cycle, the PTH-derivative was detected by RP-HPLC. PTH-Cit eluted at exactly the same position as the unidentified amino acid on position 5 in natural CXCL8(1-77), i.e. in between PTH-Thr and PTH-Gly (FIG. 1A). This confirms that the fifth amino acid in part of the natural PBMC-derived CXCL8 is posttranslationally modified from Arg into Cit. Since the experimentally determined Mᵣ of natural CXCL8 did not significantly differ from the theoretical Mᵣ and PTH-Arg₁₁ was routinely detectable, it is expected that other Arg residues present in the CXCL8 structure are not converted to Cit. Simultaneous replacement of all Arg residues by Cit was excluded based on the mass spectrometry data on more than 20 fractions containing CXCL8 immunoreactivity from 6 different C8 RP-HPLC columns (Table I). The relative amount of CXCL8(1-77) with Cit₅ instead of Argₛ per RP-HPLC fraction varied between 0% and 100% as evidenced by Edman degradation on all individual fractions. In none of these fractions Arg₁₁ of natural CXCL8(1-77) was modified. Overall 14% of natural CXCL8(1-77) was citrullinated on position 5. The % Cit₆ was calculated from the yield of the fifth cycle of the Edman chemistry. This detailed analysis evidenced that CXCL8 (1-77) and CXCL8 (1-77)Cit₅ were only partially separated on cation exchange and CO RP-HPLC. Compared to CXCL8 (1-77), CXCL8(1-77)Citₛ eluted at slightly lower NaCl concentrations from the cation exchange column and at faintly higher acetonitrile concentrations from the CS RP-HPLC column (Table I). Since Cit and Arg only differ in one mass unit, the experimentally determined mass could not be used as a criterion to predict the % citrullination. This also indicates that in body fluids, citrullination of CXCL8 can not be detected at present by the proteomic approach using ion trap mass spectrometry, and that natural proteins need to be purified to homogeneity prior to identification of this modification by Edman degradation.

Citrullination of Proteins by Peptidylarginine Deiminase (PAD)

The PAD enzymes catalyze the posttranslational hydrolysis of the guanidino group of Arg in proteins resulting in a Cit in the primary structure of these proteins (FIG. 1A). The purity of the commercial PAD preparation was verified by SDS-PAGE. Two bands were visible after proteins were blotted on PVDF membranes and stained with Coommassie blue, one corresponding to BSA and the other to PAD as evidenced by Edman degradation (data not shown). Recombinant CXCL8(1-77) was incubated with PAD at a 1:20 or 1:200 enzyme/substrate (E/S) molar ratio. Amino acid sequencing revealed that PAD converted the Arg on position 5 (Arg₅) into Cit within 30 min at a 1:200 E/S molar ratio (FIG. 2). As evidenced by Edman degradation and mass spectrometry analysis only this NH₂-terminally located Arg and no other Arg residues were citrullinated. At a 1:20 E/S molar ratio, the conversion of Argₒ occurred even more rapidly, and only Cit₅ but neither Arg₅ nor Cit₁₁ were detectable after 5 min (FIG. 2). Although 1 mM dithiothreitol (DTT) is reported to be added in most incubations of proteins with PAD (Kinloch, A., et al., 2005. Arthritis Res. Ther. 7:R1421-R1429), OTT was not essential for the conversion of Arg₅ in CXCL8 into a Cit, indicating that this Arg is highly accessible to the enzyme without partial disruption of the secondary structure of the protein. Recently, recombinant human PAD2 and PAD4 became commercially available. At a 1:200 E/S molar ratio and without addition of reducing reagents CXCL8(1-77) was found to be citrullinated by both human PAD2 and PAD4 with a half life for Arg₅ of 12 min and 15 min, respectively (FIG. 2). Thus, both human enzymes converted CXCL8(1-77) into CXCL8(1-77)Cit₅ with an efficiency comparable to rabbit PAD, also without modification of Arg₁₁ in the CXCL8 sequence. To investigate whether the citrullination was cytokine specific, recombinant IL-1β was incubated with PAD at a 1:20 E/S ratio, desalted and subjected to Edman degradation as a control. The Arg at position 4 in the protein sequence of mature biologically active IL-1β has been reported to be involved in receptor interactions. However, no citrullination was observed on either of the first two Arg of IL-1β (positions 4 and 11 in the protein sequence of activated IL-1β). Moreover, on ion trap mass spectrometry an average Mᵣ for PAD-treated IL-1β of 17375 was detected which is comparable to the theoretical average Mᵣ of IL-1β (i.e. 17377). Although PAD was not able to citrullinate IL-1β, incubation of other chemokines with an Arg present in their NH₂-terminal region (CXCL5, CCL17 and CCL26) was done with rabbit PAD, human PAD2 or PAD4. This resulted at the best tested E/S ratios and incubation times in 90 to 100% citrullination of the first NH₂-terminally located Arg as evidenced by Edman degradation (Table II). However, CXCL5 and CCL26 were citrullinated more slowly compared to CXCL8. Human PAD4, but not human PAD2, citrullinated the first NH₂-terminal Arg in CCL17 with an efficiency comparable to that in CXCL8. Moreover, the second Arg in CCL17 was also partially citrullinated by human PAD4 as well as by PAD2. In analogy with CXCL8, no conversion of the second and only other Arg was detected in CXCL5, even with 10-fold higher enzyme concentrations. In conclusion, citrullination does not appear to be a general phenomenon for all cytokines and in addition occurs preferentially on a specific position in the CXCL8 sequence which is in accordance with the posttranslational modification on natural CXCL8 (vide supra).

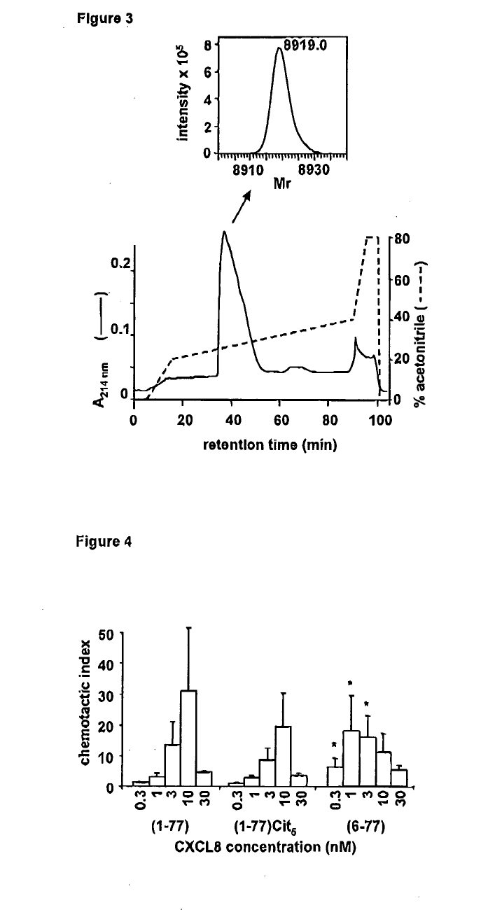

In order to obtain sufficient amounts of pure citrullinated chemokine for bioassays, 75 μg of recombinant CXCL8 was incubated with PAD at a 1:20 E/S molar ratio for 90 min and purified by C8 RP-HPLC. Modified CXCL8 eluted in one major peak from the column with a Mᵣ of 8919.0 corresponding to the theoretical Mᵣ of 8919.4 (FIG. 3). The presence of Cit₅ and Arg₁₁ was confirmed by Edman degradation. No remaining CXCL8 containing Arg₅ was detected.

Effect of Citrullination on the in vitro Chemotactic Activity and Receptor Signaling Properties of CXCL8

Authentic CXCL8(1-77), CXCL8(1-77) with Arg₅ modified to citrulline [CXCL8(1-77)Cit₆] and truncated CXCL8(6-77) were compared for their ability to attract neutrophils in the Boyden chamber assay. Although the chemotactic response to CXCL8(1-77) was moderately higher than to CXCL8(1-77)Cit₅, no significant difference in activity was detected (FIG. 4). In contrast, CXCL8(6-77) provoked a significantly better chemotactic response in comparison with CXCL8(1-77)Cit₅ and CXCL8(1-77). Truncated CXCL8(6-77) reached its maximal chemotactic activity at a concentration of 1 nM which was 10-fold lower than the optimal concentration of CXCL8(1-77) and CXCL8(1-77)Cit₅.

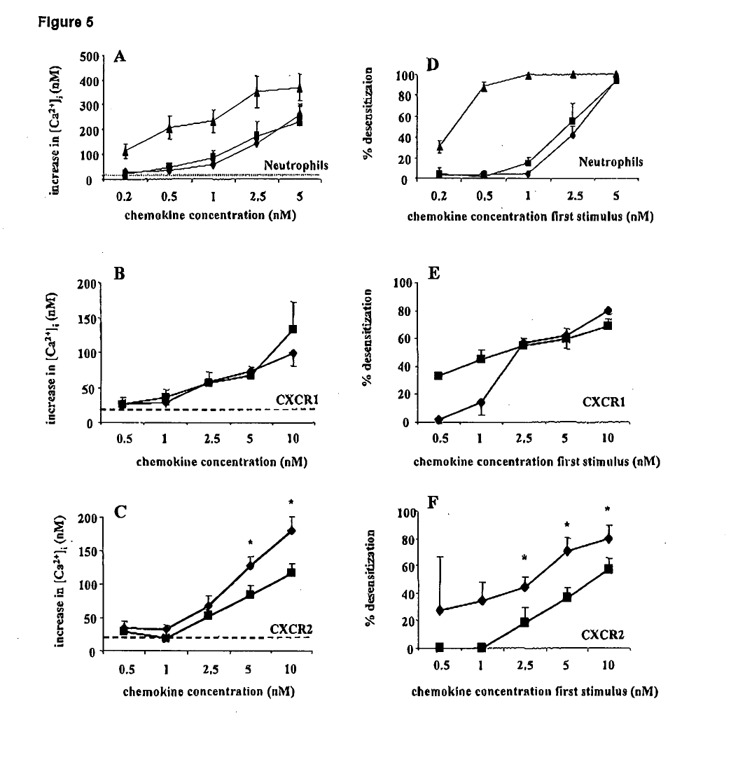

In the calcium signaling assay on neutrophils, CXCL8(1-77) and CXCL8(1-77)Cit₅ provoked a comparable increase of the [Ca^{2+]}ᵢ. As expected, CXCL8(6-77) was five-fold more potent (FIG. 5A). In addition, CXCL8(6-77) was five- to ten-fold more potent in comparison with intact unmodified and citrullinated CXCL8 at desensitizing CXCL8(1-77) (FIG. 5B).

Since CXCL8 is able to signal through two G protein-coupled chemokine receptors, i.e. CXCR1 and CXCR2 on neutrophils, the signaling capacity of CXCL8(1-77)Cit₅ was also evaluated on receptor-transfected cells. On CXCR1-transfected cells the calcium signaling capacity and potency to desensitize CXCR1 was comparable for CXCL8(1-77) and CXCL8 (1-77)Cit₅ (FIGS. 5C and D). However, on CXCR2-transfected cells, citrullinated CXCL8(1-77) was about 2- to 5-fold less potent in comparison with unmodified CXCL8(1-77) in provoking a calcium release and in desensitizing CXCR2 (FIG. 5E and F). Altogether, these results indicate that—although in vitro chemotaxis of dual receptor-bearing neutrophils is not altered—signal transduction through CXCR2 is significantly altered by citrullination of CXCL8.

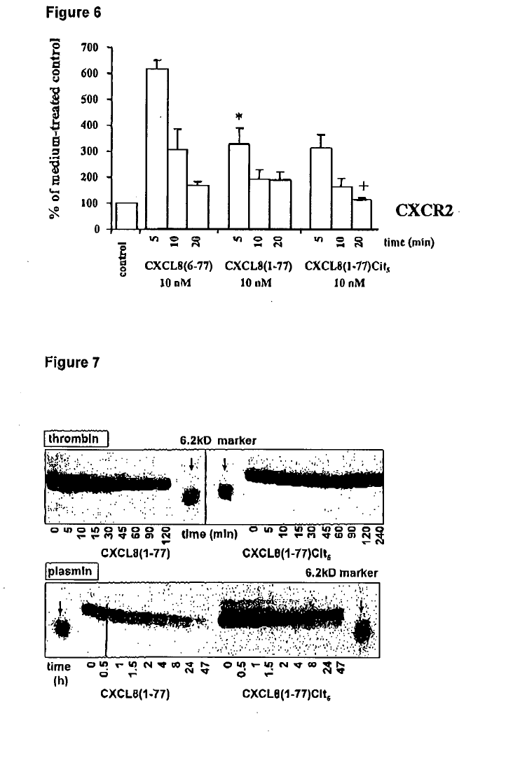

Further investigation on CXCR2 signaling was performed by measuring the amount of phosphorylated ERK1/2. Truncated CXCL8(6-77), intact CXCL8(1-77) and CXCL8(1-77)Cit₅ provoked significant phosphorylation of ERK1/2 at a concentration of 10 nM (FIG. 6). ERK1/2 phosphorylation induced by CXCL8(1-77)Cit₅ was significantly lower than CXCL8(6-77)-induced ERK1/2 phosphorylation after 5, 10 and 20 min of stimulation whereas CXCL8(1-77)-induced stimulation only differed from CXCL8(6-77) after 5 min. Moreover, a significantly higher ERK1/2 phosphorylation with CXCL8(1-77) compared to citrullinated CXCL8(1-77)Cit₅ was also observed after stimulating the cells for 20 min (FIG. 6). Thus, both the calcium and ERK1/2 signaling assays on CXCR2-transfected cells indicate that CXCL8(1-77)Cit₅ is weaker than authentic CXCL8 (1-77). It must be concluded that citrullination of CXCL8 results in CXCR2-specific reduced in vitro signaling potency.

### Effect of Citrullination on the NH₂-Processing of CXCL8 by Proteases

CXCL8(1-77) produced by PBMC is known to be NH₂-terminally truncated into CXCL8(1-77) by thrombin and plasmin. Moreover, this truncation had a more significant impact on CXCR1 or CXCR2 dependent signaling and in vitro chemotaxis compared to citrullination of Arg₅ (FIG. 4-6). Since the major cleavage site in CXCL8 for the serine proteases thrombin and plasmin is located between Arg₅ and Ser₆, the effect of posttranslational modification of this Arg to Cit on the sensitivity of CXCL8 to both proteases was investigated. As expected, recombinant intact CXCL8(1-77) was completely converted into CXCL8(6-77) by thrombin within 60 min at an E/S ratio of 1/40 as detected by ion trap mass spectrometry (data not shown). Also plasmin was able to cleave CXCL8 (E/S ratio of 1/100), but plasmin was less proficient compared to thrombin since only 40% of CXCL8(1-77) was processed within 5 h. In contrast to CXCL8(1-77), CXCL8(1-77)Cit₅ was completely resistant to thrombin as shown by Edman degradation and it was also cleaved at a much lower rate with plasmin (FIG. 7). Indeed, a smaller peptide appeared on the gel upon incubation of CXCL8(1-77)Cit₅ with plasmin for 4 h or more. However, in this experiment plasmin did not cleave CXCL8(1-77)Cit₅ into CXCL8(6-77), but into CXCL8 (9-77) and CXCL8(9-72) as evidenced by mass spectrometry. It can be concluded that Arg5 citrullination in intact CXCL8(1-77) protects this chemokine from being processed into the more active inflammatory form, i.e. CXCL8(6-77).

### Effect of Citrullination on Receptor and GAG Binding Properties of CXCL8

Cells transfected with the high affinity CXCL8 receptors were used to compare the binding efficiency of CXCL8 isoforms on CXCR1 and CXCR2. CXCL8(6-77) competed more efficiently than CXCL8(1-77) for binding of iodinated CXCL8 to CXCR1 (FIG. 8A). Despite the absence of a significant difference in calcium signaling capacity through CXCR1 (FIG. 5C), CXCL8(1-77)Cit₅ was a more potent competitor for CXCL8 binding compared to CXCL8(1-77) (FIG. 8A). In contrast, on HEK cells transfected with CXCR2 citrullination of Arg₆ did not affect the binding efficiency (FIG. 8B). Removal of the five NH₂-terminal amino acids resulted in increased CXCL8-binding on CXCR2. This indicates that the absence of the positive charge on the fifth amino acid in CXCL8(1-77) enhances the binding efficiency of this chemokine on CXCR1. To obtain a comparable increase in binding efficiency on CXCR2, truncation of the five NH₂-terminal amino acids is required. In addition to CXCR1 and CXCR2, CXCL8 also binds to the Duffy antigen receptor for chemokines (DARC) on red blood cells. CXCL8(6-77) inhibited ¹²⁵I-CXCL8 binding to red blood cells more efficiently compared to CXCL8(1-77) but citrullination of Arg₆ in CXCL8 resulted in reduced binding efficiency (FIG. 8D). In accordance with previously reported data, CCL2 but not CCL5 was able to displace ¹²⁵I-CXCL8 from DARC on red blood cells.

In addition to binding to 7-transmembrane spanning G protein-coupled receptors, CXCL8 interaction with GAG is essential for in vivo biological activity. Since chemokine binding to GAG primarily depends on positively charged amino acids and because citrullination induces a reduction of the chemokine charge, binding of CXCL8(1-77), CXCL8(1-77)Cit₅ and CXCL8(6-77) to GAG was compared on EpranEx plates treated with heparin or heparan sulfate. Although the first Arg in the CXCL8 sequence is missing in CXCL8(6-77), CXCL8(1-77) and CXCL8(6-77) had a comparable affinity for heparin and heparin sulfate (FIG. 9). In contrast to truncation, citrullination of the first Arg provoked reduced binding properties on heparin and heparan sulfate. About 3-fold higher concentrations of CXCL8(1-77)Cit₅ compared to CXC8(1-77) and CXCL8(6-77) were required to obtain a comparable signal in the GAG binding assays.

### Effect of Citrullination on the Expression of Adhesion Molecules on Neutrophils in Total Blood

Chemotaxis implements coordinate action of selectins, integrins and chemotactic factors and chemokines are known to upregulate integrin-dependent adhesion. The effect of CXCL8 isoforms on the expression on CD16⁺ neutrophils of L-selectin (CD62L), Integrins (CD11b) and the moiety that interacts with P-selectin and E-selectin (i.e. sialyl Lewis X/CD15) was determined. In addition to adhesion molecule expression, also the expression of CD16 was evaluated. FACS analysis revealed that the expression of the low-affinity receptor for the Fc region of IgG (CD16 or FcγRIII) was significantly increased upon treatment of neutrophils with CXCL8(1-77), CXCL8(6-77) or CXCL8(1-77)Cit₅. Surprisingly, CXCL8 (1-77)Cit₅ was significantly more potent than CXCL8(1-77), but less active compared to CXCL8(6-77) (FIG. 10A). The minimal effective concentrations for significant upregulation of CD16 expression (p<0.01) were 1 nM, 10 nM, and 3 nM, respectively for CXCL8(6-77), CXCL8(1-77) and CXCL8(1-77)Cit5. At 100 nM, CXCL8(1-77) provoked a significant decrease of L-selectin and at 10 nM an increase of CD15 expression on CD16⁺ neutrophils (FIG. 10B and FIG. 10C). Significantly enhanced numbers of CD16⁺/CD11b⁺ granulocytes were already obtained upon stimulation with 3 nM CXCL8(1-77) (FIG. 10D). As expected treatment of neutrophils with CXCL8(6-77) resulted in a significantly more reduced L-selectin and more increased CD11 b and CD15 expression compared to CXCL8(1-77). Citrullinated CXCL8 and CXCL8 (6-77) induced a comparable shedding of L-selectin and increase of the expression of CD15 and CD11b. In conclusion, citrullination of CXCL8 is expected to enhance leukocyte adhesion due to shedding of selectin and increase of integrin and CD15 expression but may reduce adhesion to endothelial layers due to reduced GAG-binding properties.

Effect of Citrullination on the Angiogenic Properties of CXCL8 after Local Application in vivo

CXCL8 and other CXC chemokines with ELR motif were reported to have angiogenic properties. The angiogenic activity of the different CXCL8 forms was compared in vivo in the rabbit cornea micropocket model. Hydron pellets containing different amounts of CXCL8(1-77), CXCL8(1-77)Cit₅ or CXCL8(6-77) were implanted into corneal micropockets. Maximal neovascularization occurred between day 5 and day 7 after implantation. At 3 pmol, CXCL8(1-77) and CXCL8(1-77)Cit₅ induced significant angiogenesis in comparison with control pellets, whereas CXCL8(6-77) already provoked angiogenesis at 0.3 pmol (FIG. 11). At 1 pmol CXCL8(6-77) induced maximal angiogenic activity which was significantly more elevated compared to that of CXCL8(1-77) (p<0.05) and CXCL8(1-77)Cit₅ (p<0.01). However, no variation in neovascularization was observed between CXCL8(1-77) and CXCL8(1-77)Cit₅ despite the differences in CXCR2 signaling and GAG binding (FIG. 9). These in vivo neovascularization data fully correspond with those on neutrophil chemotaxis in vitro.

### Effect of Citrullination on CXCL8-Induced Leukocyte Extravasation

In order to study the effect of citrullination of CXCL8 on leukocyte emigration from the blood circulation, CXCL8(1-77), CXCL8(6-77) or CXCL8(1-77)Cit₅ were injected i.p. in mice. Mice were sacrificed after 2 h (FIG. 12) or 4 h and leukocyte accumulation in the peritoneal cavity was evaluated. None of the three CXCL8 isoforms induced an increase in the number of peritoneal lymphocytes or macrophages. In this model, CXCL8(6-76) was three-fold more potent than CXCL8(1-77) to attract neutrophils after 2 h, confirming the observed in vitro difference in chemotactic potency between molecules (FIG. 12). In contrast to the non-citrullinated CXCL8 forms that are active at 30 pmol, up to 100 pmol CXCL8(1-77)Cit₅ was still unable to induce an increase of neither the total number nor the percentage of peritoneal neutrophils within the first 2 h. Thus, despite the lack of effect on in vitro chemotaxis assays, but in agreement with reduced GAG binding and CXCR2 signaling and resistance to NH₂-terminal proteolytic activation, citrullination of CXCL8 significantly reduced neutrophil extravasation to the peritoneal cavity. The inhibitory effect of citrullination of CXCL8 on neutrophil extravasation was even more profound compared to the enhancing effect of NH₂-terminal truncation, yielding an overall difference of 10-fold.

### Effect of Citrullination on CXCL8-Induced Leukocytosis

Whereas CXCL8 injected in tissues induces neutrophil extravasation, intravenous (i.v.) administration of CXCL8 is known to result in a rapid decrease of the number of circulating granulocytes followed by granulocytosis (van Damme J. et al. J. Exp. Med. 167:1364-1376). Recombinant CXCL8(1-77), CXCL8(1-77)Cit₅ and CXCL8(6-77) were injected i.v. in rabbits at 3 μg/kg and the concentration of circulating granulocytes and total leukocytes was determined at different time points (FIG. 13). Although the higher in vitro potency of CXCL8(6-77) compared to CXCL8(1-77) was confirmed in the in vivo cornea angiogenesis assay (FIG. 11), the hierarchy was reversed upon i.v. administration of both molecules. Indeed, CXCL8(1-77) induced a significantly more profound granulocytosis than CXCL8(6-77) upon i.v. administration in rabbits (FIG. 13). After 4 to 6 h the difference diminished, probably due to the partial conversion of CXCL8(1-77) into CXCL8(6-77) by proteases. Injection of CXCL8 (1-77)Cit₅ provoked a comparable drop in granulocyte concentration after 15 min but a two-fold more potent increase in the concentration of circulating granulocytes 2 h post-injection. Moreover, this enhanced granulocytosis induced by CXCL8(1-77)Cit₅ remained significantly more pronounced until at least 8 h post injection compared to that of CXCL8(1-77). After 24 h, the number of circulating granulocytes returned to baseline levels for CXCL8(1-77)Cit₅, CXCL6(1-77), and CXCL8(6-77). Thus, CXCL8(1-77)Cit₅ was notably more effective than CXCL6(1-77) and CXCL8(6-77) at inducing granulocytosis. Thus in contrast to the decreased extravasation of neutrophils that was observed upon injection of citrullinated CXCL8, CXCL8(1-77)Cit₅ was the most potent molecule to induce granulocytosis upon i.v. administration.

TABLE I
Identification of natural CXCL8 by Edman degradation and mass spectrometry

| Hep.Seph.^{a} | Ion exchange^{b} | RP-HPLC^{c} | | | | |
|---|---|---|---|---|---|---|
| [NaCl] | [NaCl] | [CH₃CN] | CXCL8(1-77)^{d} | CXCL8(1-77)Cit₅^{d} | Cit₅^{e} | |
| (M) | (M) | (v/v %) | (pmol/ml) | (pmol/ml) | (%) | Mᵣ^{f} |
| 0.5-0.8 | 0.61-0.64 | 30 | 5 | 3 | 40 | nd^{g} |
| | | 30.3 | 0 | 24 | 100 | nd |
| | | 30.5 | 5 | 3 | 40 | nd |
| 0.5-0.8 | 0.64-0.70 | 29.7 | 267 | 7 | 3 | 8917.6 |
| | | 30 | 333 | 36 | 10 | 8917.7 |
| | | 30.3 | 150 | 50 | 25 | 8918.8 |
| 0.5-0.8 | 0.70-0.76 | 29.7 | 125 | 0 | 0 | 8918.2 |
| | | 30 | 84 | 4 | 5 | 8917.1 |
| 0.8-1.1 | 0.58-0.62 | 30.7 | 12 | 12 | 50 | nd |
| | | 31 | 17 | 34 | 67 | 8919.0 |
| | | 31.3 | 2 | 35 | 95 | nd |
| | | 31.6 | 0 | 42 | 100 | nd |
| 0.8-1.1 | 0.62-0.65 | 30.7 | 1400 | 48 | 3 | 8918.0 |
| | | 31 | 900 | 60 | 6 | 8918.3 |
| | | 31.3 | 534 | 166 | 24 | 8917.9 |
| | | 31.6 | 214 | 286 | 57 | 8918.6 |
| | | 31.8 | 268 | 92 | 26 | 8918.7 |
| | | 32 | 56 | 64 | 53 | 8919.0 |
| 0.8-1.1 | 0.65-0.68 | 31 | 460 | 0 | 0 | 8918.1 |
| | | 31.3 | 800 | 0 | 0 | 8918.2 |
| | | 31.6 | 580 | 16 | 3 | 8917.6 |
| | | 31.8 | 180 | 6 | 3 | 8918.4 |
| | | 32 | 82 | 8 | 9 | 8919.0 |

overall % CXCL8(1-77)Cit₅ 14 %

^{a}Proteins eluting at pH 7.4 from the heparin Sepharose affinity column within the indicated molar concentrations of NaCl were pooled and further purified by cation exchange chromatography.
^{b}Proteins eluting at pH 4.0 from the Mono S cation exchange chromatography column within the indicated molar NaCl concentrations were pooled and further purified by C8 RP-HPLC
^{c}Proteins eluting from the C8 RP-HPLC column at the indicated CH₃CN concentrations (v/v %) were subjected to Edman degradation and ion trap mass spectrometry
^{d}Determined by Edman degradation
^{e}Percentage of CXCL8(1-77)Cit₅ of total CXCL8(1-77) as determined by Edman degradation
^{f}Determined by ion trap mass spectrometry
^{g}nd = not detected

TABLE II
PAD incubation of CXC and CC chemokines

| | | | | incuba- | % | % | |
|---|---|---|---|---|---|---|---|
| | NH₂-terminal | | | tion | first | second | Mᵣ |
| chemokine | sequence^{a} | enzyme | E/S^{b} | time^{c} | Cit^{d} | Cit^{d} | shift^{e} |
| CXCL5 | AGPAAAVLRELRCVC- | rabbit | 1/20 | 90 | 89.8 | 0 | +1.0 |
| | | PAD | | | | | |
| | | hu PAD2 | 1/200 | 120 | 14.0 | 0 | +0.4 |
| | | hu PAD4 | 1/200 | 120 | 35.1 | 0 | +0.6 |
| CXCL8 | AVLPRSAKELRCQC- | rabbit | 1/20 | 90 | 100 | 0 | +1.1 |
| | | PAD | | | | | |
| | | hu PAD2 | 1/200 | 120 | 88.0 | 0 | n.d. |
| | | hu PAD4 | 1/200 | 120 | 94.5 | 0 | +1.0 |
| CCL17 | ARGTNVGRECC- | hu PAD2 | 1/200 | 120 | 24.8 | 22.6 | +0.8 |
| | | hu PAD4 | 1/200 | 120 | 92.1 | 54.3 | +2.4 |
| CCL26 | TRGSDISKTCC- | hu PAD2 | 1/200 | 120 | 17.1 | n.d. | +0.2 |
| | | hu PAD4 | 1/200 | 120 | 17.8 | n.d. | +0.2 |
| | | hu PAD4 | 1/40 | 120 | 100 | n.d. | +1.3 |

^{a}NH₂-terminal Arg residues are indicated in bold, cysteine motifs are underlined.
^{b}Molar Enzyme/Substrate ratio
^{c}Incubation time in minutes
^{d}Percentages conversion of Arg residues to Cit are determined by Edman degradation
^{e}Shift compared to the theoretical average Mr of the uncitrullinated chemokine as determined by ion trap mass spectrometry
^{f}n.d. = not determined

TABLE III
Edman degradation after proteolysis of CXCL8.

| chemokine | enzyme | incubation time | | sequence after incubation | % recovered |
|---|---|---|---|---|---|
| CXCL8(1-77) | thrombin | 60 | min | SAKELRX^{a} | 100 |
| CXCL8(1-77)Cit₅ | thrombin | 60 | min | AVLPBSAKEL^{b} | 100 |
| CXCL8(1-77) | plasmin | 30 | min | AVLPRSAKEL | 80 |
| | | | | SAKELRX | 20 |
| CXCL8(1-77)Cit₅ | plasmin | 30 | min | AVLPBSAKEL | 100 |
| CXCL8(1-77) | plasmin | 5 | h | AVLPRSAKEL | 60 |
| | | | | SAKELRX | 40 |
| CXCL8(1-77)Cit₅ | plasmin | 5 | h | AVLPBSAKEL | 100 |
| CXCL8 | plasmin | 20.5 | h | degraded | |

^{a}X stands for an unidentified amino acid
^{b}B stands for citrulline

### Example 2

### Citrullination of CXCL10 and CXCL11 by Peptidylarginine Deiminase: A Naturally Occurring Posttranslational Modification of Chemokines and New Dimension of Immunoregulation

### A Materials And Methods

### Reagents and Cell Lines

Recombinant human interferon-γ (IFN-γ) and CXCL10 were obtained from PeproTech (Rocky Hill, N.J., USA). Double stranded (ds) RNA polyriboinosinic:polyribocytidylic acid (polyrl:rC) and peptidylarginine deiminase (PAD) purified from rabbit skeletal muscle (200 units/mg) were purchased from Sigma-Aldrich (St. Louis, Mo., USA). Recombinant human PAD2 and PAD4 were from Modiquest Research (Nijmegen, The Netherlands) Recombinant human CXCL11 was from R&D Systems (Minneapolis, Minn., USA). Chinese hamster ovary cells transfected with CXCR3A (CHO-CXCR3) or CXCR7, kindly provided by Marc Parmentier, were cultured in HAM's F-12 medium (Lonza, Verviers, Belgium) supplemented with 10% fetal bovine serum (PBS), 1 mM sodium pyruvate (Gibco, Invitrogen, Carlsbad, Calif., USA) and 400 μg/ml geneticin (Gibco). Human astroglioma U87 cells transfected with human CXCR3 (U87-CXCR3) were kindly provided by D. Schols and cultured as previously described (Hatse S. et al. J Virol., 2007, 81:3632-3639).

### Leukocyte Isolation and Production of Natural Chemokines

Fresh human buffy coats acquired from the blood transfusion center of Leuven were utilized to isolate leukocytes as previously described (Proost P. et al. J. Immunol., 1993, 150:1000-1010). In brief, erythrocytes were removed by sedimentation in hydroxyethyl-starch (Plasmasteril, Fresenius, Bad Homburg, Germany). Mononuclear cells and granulocytes were segregated by gradient centrifugation on Ficoll-sodium metrizoate (Lymphoprep, Nycomed, Oslo, Norway). Activated T cells were obtained by stimulating the purified peripheral blood mononuclear cells (PBMCs) from individual donors with 2 μg/ml phytohemoagglutinin (PHA; Sigma-Aldrich) for 2 to 5 days in RPMI 1640 enriched with 10% FBS and 0.05% (w/v) gentamycin (Invitrogen). Subsequently, the cells were washed with medium and cultured for 10 days in the presence of 50 U/ml human recombinant interleukin-2 (IL-2; PeproTech). T cell chemotaxis, calcium signaling and receptor binding experiments were performed two to three weeks after PHA-activation of the PBMCs and two days after the last IL-2 stimulation. Alternatively, for chemokine production, PBMCs from 24 buffy coats were pooled (11.4×10⁹ PBMCs) and induced at 5×10⁶ cells/ml with 10 μg/ml of polyrl:rC and 20 ng/ml interferon-γ (IFN-γ) in RPMI 1640 (Lonza) containing 2% FBS, The conditioned media were stored at −20° C. until purification.

### Purification of Natural Chemokines

A four-step purification procedure was performed in order to purify natural human chemokines to homogeneity. In brief, leukocyte-derived conditioned media were first concentrated to controlled pore glass by adsorption, Next, the concentrated proteins were further purified by heparin-Sepharose affinity chromatography and Mono S cation exchange chromatography (GE Healthcare, Diegem, Belgium). A specific CXCL10 sandwich ELISA was carried out to determine the chemokine concentrations in column fractions as previously described (Proost P. et al. Arthritis Res. Ther., 2006, 8:R107). Finally the CXCL10 containing fractions were subjected to reversed phase (RP-)HPLC (2.1×220 mm Brownlee C8 Aquapore RP-300 column, PerkinElmer) to acquire homogeneously purified chemokine. Proteins were eluted from the column in an acetonitrile gradient in 0.1% trifluoroacetic acid (TFA) and U.V. absorption was monitored at 214 nm. The RP-HPLC column effluent containing natural CXCL10 was split (1/150) on-line to an electrospray ion trap mass spectrometer (MS; Esquire LC, Bruker, Bremen, Germany) and collected in 400 μl fractions. Purity of the fractions was evaluated by SDS-PAGE under reducing conditions on Tris/tricine gels and proteins were visualized by silver staining.

### Identification of Posttranslational Modifications

During the elution of the proteins from the RP-HPLC column profile MS spectra were collected every 0.1 second and averaged profile spectra were calculated over the U.V. absorption peaks that contained CXCL10 immunoreactivity. The measured average Mᵣ was compared with the theoretical average Mᵣ of CXCL10 calculated using the known primary structure of CXCL10. The NH₂-terminal sequence of the isolated chemokines was determined on parallel RP-HPLC fractions by Edman degradation on a 491 cLC Procise protein sequencer (Applied Biosystems, Foster City, Calif.).

In vitro Citrullination of CXCL10 by PAD

Recombinant CXCL10 (100 pmol) was incubated with rabbit PAD (in 40 mM Tris-HCl, pH 7.4, 2 mM CaCl₂ for 2, 5, 10 and 30 minutes at 37° C. at an enzyme-substrate ratio (E/S) of 1;20. Alternatively, CXCL10 was incubated with human PAD2 or human PAD4 in 40 mM Tris-HCl, pH 7.4, 2 mM CaCl₂ at an enzyme-substrate ratio (E/S) of 1/200. Deimination was stopped by adding 0.1% TFA and samples were split and desalted on C4 ZipTip (Millipore, Billerica, Mass., USA) prior to MS or spotted on PVDF membranes (ProSorb; Applied Biosystems) prior to Edman degradation. For use in bioassays, a large amount of recombinant CXCL10 was incubated with rabbit PAD for 90 minutes at an enzyme-substrate ratio (E/S) of 1:20 and citrullinated CXCL10 was purified by RP-HPLC on a C8 Aquapore RP-300 column (1×50 mm) where 2% of the flow was converted on-line to the MS.

In vitro Chemotaxis Assays

CHO-CXCR3 cells or T cells were suspended at 2×10⁶ cells/ml in Hanks' Balanced Salt Solution (HBSS; Gibco, Invitrogen) supplemented with 0.1% human serum albumin (HSA) and added to the upper wells of a Boyden microchamber (Neuro Probe, Cabin John, Md., USA). Serial dilutions of test samples were applied to the lower wells of the microchambers and separated from the upper compartments by a polyvinylpyrrolidone-free polycarbonate filter (Nuclepore, Pleasanton, Calif.) with a pore-size of 8 μm for CHO cells or a pore-size of 5 pm for T cells, For T cells the filters were precoated with 20 μg/ml of fibronectin (Gibco). The microchambers were incubated for 2 h at 37° C. for T cells or at 32° C. for CHO cells followed by fixation and staining of the membranes with Hemacolor staining solutions (Merck, Darmstadt, Germany). The cells were counted microscopically at 500× magnification in 10 oil immersion fields. The chemotactic index was determined by dividing the number of migrated cells with the number of spontaneously migrated cells towards the sample dilution buffer (HBSS +0.1% HSA).

### Signal Transduction Assays

Phosphorylation of extracellular signal-regulated kinase 1 and 2 (ERK1/2) and protein kinase B (PKB)/Akt in response to chemokine treatment was measured as previously described (Proost P. at al. Arthritis Res. Ther., 2006, 8:R107). In brief, CHO cells transfected with CXCR3 or CXCR7 were stimulated with test samples for 5 min followed by cell lysis and subsequent centrifugation. The protein concentration in the supernatant was determined by the bicinchonic acid (BCA) protein assay and the amount of ERK1/2 and PKB/Akt phosphorylation was examined by a specific ELISA for phosphorylated ERK1 and 2 (R&D Systems) (pg phospho-ERK1/2 per mg of total protein) and for phospho-Akt (R&D Systems).

Changes in intracellular calcium concentration ([Ca²⁺]ᵢ) were measured by fluorescence spectrometry on an LS50B spectrofluorimeter (PerkinElmer, Norwalk, Conn.) as previously described.¹⁶ In brief, cells were loaded with the ratiometric fluorescent dye Fura-2/AM (Molecular Probes Europe BV, Leiden, the Netherlands) for 30 min at 37° C. in the presence (for CHO and T cells) of 125 μM probenecid (ICN Biomedicals, Aurora, Ohio). The cells were washed and resuspended in HBSS containing 1 mM Ca²⁺ (+Mg²⁺), 0.1% FBS, 10 mM HEPES pH 7.0 and 125 μM probenecid (only CHO and T cells) at a concentration of 1×10⁶ CHO or U87 cells/ml or 10×10⁶ T cells/ml. Subsequently, the cells were equilibrated at a temperature of 37° C. for lymphocytes and U87 cells or at 32° C. for CHO cells. Fura-2 fluorescence was measured at 510 nm upon excitation at 340 nm and 380 nm.

### Peptide Synthesis

Intact CXCL11 and CXCL11 with a citrulline residue on position 6 were synthesized by solid phase peptide synthesis (SPPS) with fluorenylmethoxycarbonyl (Fmoc) protected α-amino groups using a 433A peptide synthesizer (Applied Biosystems) as described previously (Proost P. et al. Blood, 2007, 110:37-44). The synthetic peptides were purified on a 4.6×150 mm Source 5RPC column (GE Healthcare) and detected by U.V. absorption at 220 nm. The average Mᵣ was determined by on-line ion trap MS. The peptides with the correct Mᵣ were folded overnight at room temperature in 150 mM Tris pH 8.6 containing 1 M guanidinium chloride, 3 mM EDTA, 0.3 mM reduced glutathione and 3 mM oxidized glutathione. After a second RP-HPLC on a C-8 Aquapore RP-300 column and on-line MS, the fractions with the correct Mᵣ were pooled, the NH₂-terminal sequence was confirmed by Edman degradation and the protein concentration was determined using the bicinchonic acid (BCA) protein assay (Pierce Biotechnology, Rockford, Ill., USA).

### Binding Assays

Receptor binding properties were determined by competition for ¹²⁵I-labeled CXCL10 or CXCL11 binding on CHO-CXCR3 or CHO-CXCR7 cells and on T cells as previously described.¹⁵ In brief, 2×10⁶ cells were incubated for 2 h at 4° C. with ¹²⁵I-labeled (GE Healthcare) and unlabeled chemokine. Subsequently, the cells were centrifuged, washed three times and radioactivity was measured. Heparin binding was assessed following the manufacturers instructions by immobilizing 25 μg/ml of low molecular weight heparin (Sigma-Aldrich) diluted in phosphate-buffered saline (PBS) overnight at room temperature on EpranEx plates (Plasso Technology Ltd., Sheffield, UK). The plates were then washed three times with standard assay buffer (100 mM NaCl, 50 mM NaAc, 0.2% (v/v) Tween-20 pH 7.2) and blocked at 37° C. with standard assay buffer enriched with 0.2% (w/v) gelatin. The heparin-captured chemokines were detected with 0.16 μg/ml biotinylated anti-human CXCL10 or CXCL11 (PeproTech) and peroxidase conjugated streptavidin (Jackson ImmunoResearch Laboratories, West Grove, Pa.). Peroxidase activity was quantified by measuring the conversion of 3,3′,5,5′-tetramethylbenzidine (Sigma-Aldrich) at 450 nm. No difference in specificity of the biotinylated antibodies for the chemokines and their citrullinated isoforms was uncovered. Percentage binding was calculated as a result of standardizing the optical density (OD) by subtracting the mean OD of the negative control (blocking buffer) from the measured OD of the sample, subsequent division with the average OD of the highest concentration of intact chemokine and finally multiplying by 100. This calculation grants a value of 100% binding to the highest concentration of intact chemokine and 0% binding to the negative control.

In vitro Wound Healing Assay

Inhibition of endothelial cell migration was assessed as previously described (Proost P. et al. Blood, 2007, 110:37-44). Briefly, human dermal/skin microvascular endothelial cells (HMVECs) were grown to confluency in EBM-2 medium enriched with EGM-2-MV Bulletkit (Lonza). A linear scar was drawn in the monolayer and series of chemokine dilutions and control medium were added to the cells. Differences in width before and after 24 hours of treatment were examined under a microscope double-blind by two investigators and migration scores were acquired by setting the variation in optical scores in medium-treated cultures (control) to zero. Scars broader than the control were given a negative score indicating inhibition of migration.

### Heparin Binding Assay

Heparin binding was assessed following manufacturer's Instructions by immobilizing 25 μg/m1 of low molecular weight heparin (Sigma-Aldrich) diluted in phosphate-buffered saline (PBS) overnight at room temperature on EpranEx plates (Plasso Technology Ltd., Sheffield, UK). The plates were then washed three times with standard assay buffer (100 mM NaCl, 50 mM NaAc, 0.2% (v/v) Tween-20 pH 7.2) and blocked at 37° C. with standard assay buffer enriched with 0.2% (w/v) gelatin. The heparin-captured chemokines were detected with biotinylated antihuman CXCL10 or CXCL11 (PeproTech) and subsequently by peroxidase conjugated streptavidin (Jackson ImmunoResearch Laboratories, West Grove, Pa.). Peroxidase activity was quantified by measuring the conversion of 3,3′,5,5′-tetramethylbenzidine (Sigma-Aldrich) at 450 nm. No difference in specificity of the biotinylated antibodies for the intact chemokines and their isoforms was uncovered. Percentage binding was calculated by standardizing the optical density (OD) by subtracting the mean OD of the negative control (blocking buffer), subsequent division with the average OD of the highest concentration of intact chemokine and finally multiplying by 100. This calculation grants a value of 100% binding to the highest concentration of intact chemokine and 0% binding to the negative control.

### B Results

### Identification of Naturally Citrullinated Isoforms of CXCL10

Natural CXCL10 was purified from stimulated PBMCs by subsequent heparin affinity chromatography, cation exchange and C8 RP-HPLC (Proost P. at al. J Immunol. 1993;150:1000-1010). At each chromatographical step CXCL10 immunoreactivity was determined by specific ELISA and pure proteins corresponding to CXCL10 immunoreactivity were identified by Edman degradation. Since Cys residues were not alkylated prior to amino acid sequencing, the phenyl thiohydantoin (PTH)-Cys residues were not detected. Upon NH₂-terminal sequencing an unidentified compound eluted from the RP-HPLC column in between PTH-Thr and PTH-Gly instead of a predicted PTH-Arg peak at position 5 (PTH-Arg₅) for natural CXCL10 (FIG. 14A). This compound was only detected in certain CXCL10 fractions, whereas the PTH-Arg₈ was present in all proteins corresponding to CXCL10 immunoreactivity. The experimentally determined Mᵣ of this naturally modified CXCL10 did not significantly differ from the theoretical Mᵣ of CXCL10. A possible posttranslational modification of Arg which could result in such an insignificant alteration in Mᵣ (one mass unit) is the conversion of Arg into citrulline (Cit). Edman degradation on pure L-citrulline resulted in a PTH-derivative that eluted at exactly the same position as the unidentified amino acid on position 5 in the purified CXCL10 fractions, i.e. in between PTH-Thr and PTH-Gly (FIG. 14B). Thus, the unidentified fifth amino acid present in the sequence of natural PBMC-derived CXCL10 is indeed Cit. No further COOH-terminal citrullination was detected for natural CXCL10 as this would result in a detectable increase in Mᵣ of 2 or more mass units by ion trap MS.

### Modification of CXCL10 by Peptidylarginine Deiminase

The enzymatic hydrolysis of the guanidino group of Arg in proteins yielding a Cit incorporated in the primary structure of these proteins can be generated by peptidylarginine deiminases (PAD) (Vossenaar E R, et al. Bioessays 2003;25:1106-1118). Recombinant CXCL10 was therefore incubated with PAD at a 1:20 enzyme-substrate ratio (E/S). After 2 min, PTH-Cit was already detectable by Edman degradation at position 5 in the CXCL10 sequence (FIG. 15A). After 10 min of enzyme incubation, 50% of the chemokine was modified on the first Arg and after 30 min only PTH-Cit was detected. Alternatively, recombinant CXCL10 was incubated with human PAD2 or human PAD4 at an E/S ratio of 1:200. Both PAD2 and PAD4 converted Arg₅ into Cit with equal efficiency confirming the data obtained with rabbit PAD. At a 1:200 E/S ratio, 50% of Arg₅ was converted into Cit without effect on Arg₈.

For a detailed biological characterization of citrullinated chemokine, recombinant CXCL10 was enzymatically converted into CXCL10-Cit₅ by incubation with PAD for 90 min at a 1:20 PAD/CXCL10 molar ratio. HPLC-purified PAD-treated CXCL10 had a Mᵣ of 8618.9 corresponding to CXCL10-Cit₅ (theoretical Mᵣ of 8618.3) (FIG. 15B). The complete conversion of Arg₅ into Cit₅ and the retention of Arg₈ were confirmed by Edman degradation.

Citrullination Impairs in vitro Biologic Activity of CXCL10 on CXCR3-Tranfected Cells

The CXCR3-dependent chemotactic activity of recombinant CXCL10 and citrullinated CXCL10-Cit₅ were compared on CHO-CXCR3 cells in the Boyden microchamber assay. CXCL10 provoked a typical bell-shaped dose response with significant chemotaxis compared to buffer starting from 0.3 nM onward (FIG. 16A), whereas a maximal chemotactic index of 10 was reached at 1 nM CXCL10. Citrullinated CXCL10-Cit₅, however, was unable to induce any significant chemotactic response on CHO-CXCR3 cells. Moreover, a highly significant difference existed between the chemotactic indices obtained by recombinant and citrullinated CXCL10.

Subsequently, authentic CXCL10 and CXCL10-Cit₅ were examined for their signaling capacities. In intracellular phosphorylation assays on CHO-CXCR3 cells, CXCL10 at a concentration of 10 nM induced a significantly increased amount of phosphorylated ERK1/2 and PKB/Akt compared to medium-treated control (FIG. 16B). In contrast, CXCL10-Cit₅ was incapable of provoking any significantly enhanced phosphorylation in CHO-CXCR3 cells, resulting in a significant difference between the two isoforms. On the other hand, CXCL10-Cit₅ still caused an increase in [Ca²⁺]ᵢ in CHO-CXCR3 cells starting at a dose of 10 nM (FIG. 16C). Nonetheless, authentic CXCL10 was more potent (at least ten-fold) than CXCL10-Cit₅ since a significant increase in [Ca²⁺]ᵢ was obtained at 1 nM. A similar pattern emerged in receptor desensitization experiments (FIG. 16D). Indeed, upon rechallenging CHO-CXCR3 cells with chemokine, CXCL10-Cit₅ was observed to be about ten times less effective at inhibiting the intracellular calcium release of authentic CXCL10 in comparison with auto-desensitization. Taken together, these data demonstrate that although CXCL10-Cit₅ fails to exert chemotactic activity and induce phosphorylation, it is still capable of binding CXCR3 and to some extent preventing signal transduction by authentic CXCL10.

### Chemical Synthesis of CXCL11-Cit₆ and Its in Vitro Biologic Activity in CXCR3-Transfected Cells

To assess whether related chemokines are substrates for PAD and are affected by citrullination, we tested another T cell chemoattractant, i.e. CXCL11. Recombinant CXCL11 was first found to be also citrullinated by PAD into CXCL11-Cit₆ as confirmed by Edman degradation. To evaluate the effects of citrullation on the biological activities of CXCL11, both authentic CXCL11 and CXCL11 with a citrulline incorporated at position 6 were chemically synthesized using Fmoc chemistry as previously described (Proost P. et al. Blood 2007;110:37-44). After deprotection and purification on RP-HPLC, the Mᵣ was verified by on-line ion trap MS and by NH₂-terminal sequencing. Consequently, the chemokines were folded in buffer containing oxidized and reduced glutathione. The folded peptides were purified by RP-HPLC and the formation of the disulfide bridges was confirmed by on-line mass-spectrometry. Synthetic and recombinant intact CXCL11 were shown to be equally potent in chemotaxis and calcium signaling assays, indicating a successful peptide synthesis and folding procedure (vide infra). Although authentic CXCL11 seemed to be more effective at phosphorylating ERK1/2 and PKB/Akt in CHO-CXCR3 cells compared to CXCL11-Cit₆, no statistical difference was observed between the two isoforms (FIG. 17A and 17B). However, CXCL11 was significantly more potent than CXCL11-Cit₆ (about three-fold) at inducing an increase in [Ca²⁺]ᵢ in U87-CXCR3 cells (FIG. 17C). These data suggest an important role for citrullination on the biological effect of CXCL11 through CXCR3, however less pronounced than for CXCL10.

Citrullination does not Interfere with Binding Properties of CXCL10 and CXCL11 on CXCR3 and CXCR7

To further characterize the properties of citrullinated CXCL10 and CXCL11, receptor binding studies were performed. Surprisingly, CXCL10 and CXCL10-Cit₅were equally potent at dislocating ¹²⁵I-labeled CXCL10 from CHO-CXCR3 cells (FIG. 18A). Similarly, authentic CXCL11 and CXCL11-Cit₆ competed equally well for ¹²⁵I-labeled CXCL11 in binding to CXCR3-transfected cells (FIG. 18B). CXCL11 and CXCL11-Cit₆ were also alike in displacing the radiolabeled CXCL11 from CXCR7 transfectants (FIG. 18C). In addition, the capacity to compete for ¹²⁵I-labeled CXCL11 binding on T cells was similar for CXCL11 and CXCL11-Cit₆ (FIG. 18D). This indicates that the Arg at position 5 or 6 in, respectively, CXCL10 and CXCL11 and thus the change from a basic to a neutral amino acid, are not implicated in the receptor binding competition. Nevertheless, CXCL10-Cit₅ and to some extent CXCL11-Cit₆, were weaker at provoking biological activities through CXCR3 (FIGS. 16 and 17).

### Citrullination Reduces T Cell Activities of CXCL10 and CXCL11

The biological activities of citrullinated and unmodified CXCR3 ligands were compared under more physiological conditions, i.e. on activated 7 lymphocytes. CXCL10-Cit₅ was less potent (minimal effective concentration of 20 nM) at provoking chemotaxis of 7 cells (FIG. 19A). In contrast to the chemotactic response observed on CHO-CXCR3 cells (FIG. 16), however, CXCL10-Cit₅ remained active on T cells. In calcium signaling experiments, CXCL10 already induced an increase in [Ca²⁺]ᵢ starting from 3 nM onward, whereas CXCL10-Cit₅ only exceeded the detection limit at 30 nM (FIG. 19B), resulting in a statistically significant difference between the two isoforms, as was observed for signaling in CHO-CXCR3 transfectants (FIG. 16). CXCL11-Cit₆ was also shown to stimulate T cell chemotaxis (FIG. 19C) but the minimal effective concentration (3 nM) was slightly (3-fold) higher than for authentic CXCL11. For comparison, synthetic and recombinant CXCL11 did not differ in chemoattraction of T cells, confirming a successful chemical synthesis. Furthermore, in the calcium signaling experiments, T cells responded significantly less to CXCL11-Cit₆ than to authentic CXCL11 (FIG. 19D). Indeed, unmodified CXCL11 induced increases in [Ca^{2+]}ᵢ from 1 nM onward, whereas for CXCL11-Cit₆ 10 nM was required. Similarly, CXCL11 desensitized the calcium response more efficiently than CXCL11-Cit₆ (FIG. 19E). These data are in agreement with the observation that the calcium signaling response of CXCL11-Cit₆ in CXCR3 cells was reduced compared to authentic CXCL11 (FIG. 17C). Thus, despite the fact that no differences for these CXCL11 forms could be observed in their binding properties to CXCR3 or CXCR7 transfected cells and to T cells (FIG. 18) nor in their CXCR3 dependent phosphorylation (FIG. 17A and B), these molecules do differ in chemoattraction and calcium signaling in CXCR3 transfected cells and in T lymphocytes. Moreover, . no CXCR7 mRNA could be detected on our T cells by RT-PCR. CXCR3 expression on T cells, however, was confirmed by FACS analysis.

### Citrullination Impairs GAG Binding of CXCL10 and CXCL11

As a final approach to obtain more insight in the discrepancies observed in biological activities between authentic and citrullinated CXCL10 and CXCL11, we investigated glycosaminoglycan (GAG) binding properties. GAGs are sulfated polysaccharides that form part of the glycocalyx on cell surfaces and are imperative in the sequestration of chemokines as well as the formation of a chemokine gradient. GAG-chemokine binding may also modulate the presentation of the chemokine to its receptor and hence affect its biological activity. Interestingly, the heparin binding capacity of CXCL10-Cit₆ was significantly diminished by citrullination in comparison with authentic CXCL10 (FIG. 20A). Additionally, citrullination decreased the heparin binding capacity of CXCL11 (FIG. 20B). These data reveal a high degree of dissimilarity on, GAG binding due to citrullination of a single Arg. For further exploration, citrullinated CXCL11 was evaluated in an in vitro wound healing assay. CXCL11 and CXCL11-Cit₆ were both observed to significantly inhibit the migration of human microvascular endothelial cells (HMVECs) in an equally potent manner (FIG. 20C).

### Example 3

### Citrullination of CXCL12 Impairs its CXCR4 and CXCR7 Binding Capacity

### A Materials and Methods

### Reagents and Cells

Recombinant CXCL12, and synthetic CXCL12 that was C-terminally fluorescently labeled with Alexa Fluor 647 (CXCL12^{AF647}) were obtained from R&D Systems (Abingdon, U.K.), and Aimee Sciences (East Lothian, Scotland, U.K.), respectively. Peptidylarginine deiminase (PAD) purified from rabbit skeletal muscle was purchased from Sigma-Aldrich (St. Louis, Mo.).

Synthetic CXCL12 isoforms were prepared by fluorenylmethoxycarbonyl solid phase peptide synthesis with appropriate side-chain protection groups on a 431A peptide synthesizer (Applied Biosystems, Foster City, Calif., USA) as previously described. The synthetic CXCL12 forms were deprotected and cleaved from the resin for 90 min at room temperature in 10 ml TFA containing 0.75 g phenol, 0.5 ml thioanisole, 0.25 ml ethanedithiol and 0.5 ml water. Subsequently, peptides were precipitated and washed in diethyl ether, dissolved in water and loaded on a Source 5RPC column (4.6×150 mm; GE Healthcare). The proteins were eluted from the RP-HPLC column with an acetonitrile gradient in 0.1% TFA and the U.V. absorption of peptides was monitored at 220 nm. Part of the column effluent (0.7 v/v %) was split to an Esquire LC ion trap mass spectrometer (Bruker, Bremen, Germany) and averaged profile spectra were deconvoluted to determine the M. Proteins with the correct Mᵣ were folded for 2 h in 150 mM Tris pH 8.6 containing 1 M guanidinium chloride, 3 mM EDTA, 0.3 mM reduced glutathion and 3 mM oxidized glutathion and repurified by RP-HPLC on a C8 Aquapore RP-300 column (PerkinElmer). Ion trap mass spectrometry was used to select the fractions that contained the folded peptides. These fractions were pooled and the protein concentrations were determined.

Fresh peripheral blood-derived mononuclear cells (PBMC) were obtained from healthy donors and isolated by hydroxyethyl starch sedimentation and Ficoll-sodium metrizoate centrifugation. The MT-4 lymphoblastic cell line and monocytic THP-1 cells were cultured in RPMI 1640 (Cambrex, Verviers, Belgium) supplemented with 10% fetal bovine serum (FBS). Human CXCR4 or CXCR7 were stably expressed in Chinese hamster ovary K1 (CHO-K1) cells (American Type Culture Collection [ATCC], Manassas, Va.: CCL-61) and the cells were cultured in Ham's F-12 medium (Cambrex) supplemented with 10% FBS, 1 mM sodium pyruvate, and 400 μg/ml geneticin as previously described.

Chemokine Treatment with PAD

CXCL12 was citrullinated with PAD in 40 mM Tris-HCl, pH 7.6, 2 mM CaCl₂ at 37° C. The reaction was stopped by adding 0.1% ) TFA. CXCL12 treated with PAD was spotted on PVDF membranes (ProSorb; Applied Biosystems) and the NH₂-terminal sequence of the protein was determined by Edman degradation. The phenyl thiohydantoin derivates of the individual amino acids were separated by RP-HPLC on a 0.8×250 mm PTH Column (Applied Biosystems) and the height of the peaks was used to calculate the relative amount of Arg converted to Cit. The phenyl thiohydantoin derivate of citrulline (Cit) eluted between the derivates of Thr and Gly as previously described (see Example 1). In order to obtain CXCL12 with 5 citrullinated Arg for use in bioassays, synthetic CXCL12 was treated with PAD, folded and purified by C8 RP-HPLC on an Aquapore RP-300 column (1×50 mm). About 2% of the column effluent was converted to the on-line ion trap mass spectrometer.

### Detection of Chemotactic Activity

The chemotactic activity of CXCL12 for monocytes was determined in Boyden chemotaxis chambers. PBMC suspensions and samples dilutions were prepared in Hank's balanced salt solution without calcium or magnesium, supplemented with 1 mg/ml human serum albumin. All samples were tested in triplicate. PBMC (2×10⁶ cells/ml) were allowed to migrate at 37° C. for 2 h through 5 μm pore-size polycarbonate filters (GE Osmonics, Minnetonka, Minn.). Filters were removed, cells were fixed and stained and counted microscopically. Chemotactic activity for monocytic THP-1 cells was tested in 96-well Multiscreen-MIC polycarbonate plates with a pore-size of 5 μm (Millipore, Bedford, Mass.). Cells (3×10⁶ cells/ml) and samples were suspended in RPMI medium without phenol red with 0.1% bovine serum albumin (Sigma). After 3 h incubation at 37° C. and 5% CO₂ the bottom compartment of the Multiscreen-MIC plate was centrifuged at 220 g and cell migration was quantified by measuring the amount of ATP present in the cells in the bottom compartment with a luminescence detection assay (ATPlite, PerkinElmer). Results are expressed as chemotactic index (CI) corresponding to the number of cells migrated to the sample over the number of cells that migrated to control medium.

### Binding Studies

Competition for CXCL12^{AF647} binding was measured on CXCR4 or CXCR7-transfected CHO cells. Briefly, 1.5×10⁶ cells/ml were incubated for 1 h at 4° C. with 20 ng/ml CXCL12^{AF647} and varying concentrations of unlabeled chemokine in RPMI1640+2% FCS. Cells were washed 3 times with FACS buffer (PBS+2% FCS) and fixed in FAGS buffer containing 2% formaldehyde (fixation buffer). The fluorescence present on the cells was measured by flow cytometry. Aspecific binding was determined by adding a 100-fold excess of unlabelled ligand over the concentration of labelled ligand. Specific binding was obtained by subtraction of aspecific binding from total binding. Results are expressed as the % of remaining specifically bound CXCL12^{AF647}.

Binding to GAG was evaluated by immobilizing 25 μg/ml of low molecular weight heparin (Sigma-Aldrich) overnight at room temperature on 96-well EpranEx plates (Plasso Technology Ltd., Sheffield, UK). Plates were washed three times with acetate buffer (100 mM NaCl, 50 mM NaAc, 0.2% (v/v) Tween-20 pH 7.2) and blocked at 37° C. with acetate buffer enriched with 0.2% (w/v) gelatine (block buffer). Chemokines were allowed to interact with heparin for 2 h at 37° C. in block buffer and unbound chemokines were removed in three consecutive washing steps in acetate buffer. The captured chemokines on heparin treated plates were detected with biotinylated anti human CXCL12 (PeproTech Inc.) and consequently by peroxidase conjugated streptavidin (Jackson ImmunoResearch Laboratories, West Grove, Pa.). Peroxidase activity was quantified by measuring the conversion of 3,3′,5,5′-tetramethylbenzidine (TMB; Sigma) at 450 nm. The optical density (OD) with 100 nM CXCL12 minus the OD obtained in the absence of chemokine was set to 100%. No difference in specificity of the biotinylated antibody for unmodified or citrullinated CXCL12 was uncovered.

### Intracellular Signaling Assays

The intracellular Ca²⁺ concentrations [Ca²⁺]ᵢ were determined spectrofluorometrically using the fluorescent dye fura-2 and were calculated from the Grynkiewicz equation as previously described. For desensitization experiments, cells were stimulated first with native or modified CXCL12 and 100 s later with native CXCL12 at a concentration (10 ng/ml) that induced a significant increase in [Ca²⁺]ᵢ after prestimulation with buffer.

CXCL12-dependent phosphorylation of extracellular signal-regulated kinases 1 (ERK1) plus ERK2 and Akt/protein kinase B (PKB) was evaluated in receptor-transfected CHO cells as previously described. Briefly, cells were grown in 6-well plates in Ham's F-12 medium with 10% FBS for 24 h. Subsequently, the cells were cultured overnight in serum-free starvation medium followed by a pre-incubation of the cells for 15 min at 37° C. in medium with 0.5% FBS before stimulation with test sample for 5 min at 37° C. Signal transduction was stopped by chilling on ice and adding ice-cold PBS. Afterwards, cells were washed twice with ice-cold PBS and cell lysis was performed in PBS containing 1 mM EDTA, 0.5% Triton X-100, 5 mM NaF, 6 M urea, protease inhibitor cocktail for mammalian tissues and phosphatase inhibitor cocktails 1 and 2 (Sigma-Aldrich) (100 μl/well). After 10 min, cells were scraped off, the lysate collected and incubated for 45 min on ice and clarified (10 min, 1200 g). The protein concentration in the supernatant was determined by the bicinchoninic acid (BCA) protein assay (Pierce, Rockford, Ill.). The amount of ERK and PKB/Akt phosphorylation in the supernatant (in pg phospho-ERK or phospho-Akt per mg total protein) was determined using specific ELISAs from R&D Systems: CatNr DYC887-2 for phospho-Akt (S473) and CatNr DYC1018-2 recognizing both phospho-ERK1 (T202/Y204) and phospho-ERK2 (T185/Y187).

### Internalization of CXCR4

Freshly purified PBMC in preheated RPMI 1640+0.5% human serum albumin, were incubated with chemokine solutions at 37° C. After 30 min 150 μl ice-cold FACS buffer containing 15 μl PE labeled anti-human CXCR4 (BD Biosciences, San Jose, Calif., USA) or PE labeled isotype control (IgG_{2α′κ}; BD Biosciences) was added to the cells. As a negative control, FACS buffer was used in combination with unstimulated PBMC. The cells were then incubated at 4° C. for 30 minutes. After washing away unbound fluorescent label, the cells were fixed in fixation buffer and analyzed afterwards by flow cytometry. Results shown are % of remaining fluorescence.

### Detection of Antiviral Activity

Lymphocytic MT-4 cells were treated with varying concentrations of CXCL12, CXCL12-Cit₈, CXCL12-Cit_{8,12,20} or CXCL12-Cit_{8,12,20,41,47} at the time of infection with the X4-tropic NL4.3 HIV-1 strain (10⁵ pg of p24). After 1 h, non-absorbed virus was removed by washing three times with PBS and HIV-infected cells were cultured in RPMI 1640 with 10% FBS. HIV-1 titers were determined in the culture supernatant with a commercial p24 Ag ELISA (DuPont, Wilmington, Del.).

### B Results

### Citrullination of CXCL12 by PAD

Recombinant CXCL12 was treated with purified PAD and citrullination of the individual Arg residues was evaluated by Edman degradation on the first 21 NH₂-terminal amino acids of CXCL12. The conversion of Arg₈, Arg₁₂ and Arg₂₀ in the CXCL12 sequence proceeded with equal velocity indicating that these three Arg are equally accessible and sensitive for the active site of PAD (FIG. 21). After 5 min at a 1/20 enzyme/substrate ratio, as much as 50% of these Arg residues was converted in to citrulline and 90% of Arg was citrullinated after 30 min. No intact Arg (at least 97% conversion) was detectable after incubation for 45 min of CXCL12 with PAD.

### Chemical Synthesis of CXCL12 Isoforms

In order to be able to investigate the influence of specific Arg modifications in the CXCL12 protein structure, authentic CXCL12, CXCL12-Cit₈ (with Cit only on position 8) and CXCL12-Cit_{8,12,20} (with Cit on positions 8, 12 and 20) were chemically synthesized, purified by RP-HPLC and folded. The experimentally determined Mr of CXCL12 (7959.3), CXCL12-Cit₈ (7960.5) and CXCL12-Cit_{8,12,20} (7962.6) were comparable to the theoretical Mr of the three proteins, i.e. 7959.5, 7960.5 and 7962.5, respectively (FIG. 22). The presence in the synthetic proteins of Cit in position 8 (CXCL12-Cit₈) and in positions 8, 12 and 20 (CXCL12-Cit_{8,12,20}) was confirmed by, Edman degradation. In addition, a CXCL12 form with all five Arg, also including Arg41 and Arg47 modified to Cit was prepared by incubation of synthetic CXCL12 with PAD for 3.5 h, subsequent folding and RP-HPLC purification. The structure of CXCL12-Cit_{8,12,20,41,47} (FIG. 22) was confirmed by ion trap mass spectrometry (experimental Mr of 7964.8 versus theoretical Mr 7964.5) and by Edman degradation on the first 20 amino acids.

### Effect of Citrullination on the Chemotactic Activity of CXCL12

The activity of authentic CXCL12 and CXCL12 with 1, 3 or 5 Cit residues instead of Arg was compared in in vitro chemotaxis experiments on monocytic THP-1 cells and on freshly isolated blood monocytes from three different donors (FIG. 23). On THP-1 cells a maximal chemotactic response was obtained with CXCL12 at 3 nM. For CXCL12-Cit₈ a 10-fold higher dose was required to obtain a maximal response. In addition, CXCL12-Cit₈ reached a maximal chemotactic index of <6 compared to a CI of 12 for unmodified CXCL12. A comparable low chemotactic index was already reached with less than 1 nm of CXCL12. CXCL12⁻Cit_{8,12,20} and CXCL12Cit_{8,12,20,41,47} were completely devoid of any chemotactic activity for THP-1 cells at concentrations up to 100 nM. On freshly isolated PBMC, a maximal chemotactic response was obtained with 100 nM CXCL12. Comparable with the results on THP-1 cells, CXCL12-Cit₈ was significantly less chemotactic for monocytes, whereas CXCL12-Cit_{8,12,20} and CXCL12-Cit_{12,20,41,47}were again inactive.

### Effect of Citrullination on CXCL12-Mediated Signal Transduction

In CHO-CXCR4 cells CXCL12 induced a significant increase in [Ca²⁺]ᵢ from 0.3 nM onwards (FIG. 24). To obtain a similar response with CXCL12-Cit₈, about 30-fold higher amounts were required. As observed in the chemotaxis experiments, CXCL12-Cit_{8,12,20} and CXCL12-Cit_{8,12,20,41,47} were completely inactive at concentrations up to 100 nM. Compared to authentic CXCL12, CXCL12-Cit₈ was also 30-fold less efficient at desensitizing the calcium responses to 1 nM CXCL12, whereas CXCL12-C11_{8,12,20} and CXCL12-Cit_{8,12,20,41,47} failed to desensitize CXCR4.

CXCL12 has been reported to induce phosphorylation of ERK1, ERK2 and Akt/PKB. After 5 min significant ERK phosphorylation was detected when CHO-CXCR4 cells were treated with 0.03 nM CXCL12 and optimal phosphorylation of ERK was obtained upon CXCL12-Cit₈ resulted in significant phosphorylation of ERK and optimal stimulation was obtained with 30 to 100 nM of CXCL12-Cit₈ (FIG. 25). In addition to ERK also Akt/PKB phosphorylation was detected on CXCL12-treated CHO-CXCR4 cells. Akt/PKB phosphorylation was detected with 0.03 nM CXCL12 and an about 30-fold higher dose of CXCL12-Cit₈ was required to obtain a comparable Akt/PKB phosphorylation. Neither CXCL12-Cit_{8,12,20} nor CXCL12-Cit_{8,12,20,41,47} induced CXCR4-dependent ERK or Akt/PKB phosphorylation at concentrations as high as 100 nM. On CHO-CXCR7 cells, up to 300 nM CXCL12 was unable to induce ERK or Akt/PKB phosphorylation.

### Effect of Citrullination on CXCL12-Mediated Receptor and GAG Binding

Competition for CXCL12 binding to the receptors CXCR4 and CXCR7 was investigated with CXCL12^{AF}⁶⁴⁷ since two commercial preparations of ¹²⁵I-CXCL12 only weakly bound to CHO-CXCR4 cells although they efficiently and specifically bound to CHO-CXCR7 cells. CXCL12 efficiently competed for CXCL12^{AF647} binding to CXCR4 and CXCR7 (FIG. 26). Half of the labeled-CXCL12 was removed from the CHO-CXCR4 or CHO-CXCR7 cells with 4.3 nM or 7.7 nM of unlabeled chemokine, respectively. In accordance with the signaling data CXCL12-Cit₈ weakly competed (50% inhibition of binding at 50 nM CXCL12-Cit₈) and CXCL12-Cit_{8,12,20} and CXCL12-Cit_{8,12,20,41,47} failed to compete for binding to CXCR4. Surprisingly, CXCL12-Cit₈ was equally efficient compared to CXCL12 to compete for binding of CXCL12^{AF647} to CXCR7. Moreover, although CXCL12-Cit_{8,12,20,41,47} failed to bind to CXCR7, CXCL12-Cit_{8,12,20} still moderately competed for CXCL12^{AF647} binding.

In addition to G-protein coupled receptors, chemokines also bind to GAG. Since GAG binding is mainly dependent on positively charged residues, the effect of CXCL12 citrullination on the interaction with heparin was investigated. Citrullination of the first three Arg, resulting in the loss of three positive charges in the CXCL12 structure, did not affect its heparin binding properties (FIG. 27). However, further citrullination of the two more C-terminally located Arg resulted in 3-fold reduced binding to heparin.

### Effect of Citrullination on CXCL12-Mediated Internalization

G-protein coupled receptors may be rapidly internalized upon binding of active ligands. CHO-Purified PBMC were treated with unmodified or citrullinated CXCL12 for 30 min and subsequently labelled with CXCR4 specific antibodies. FACS analysis was used to quantify the amount of remaining surface bound CXCR4 on monocytes and lymphocytes. CXCR4 was less efficiently internalized with CXCL12-Cit₈ compared to CXCL12 (FIG. 28). CXCL12-Cit_{8,12,20} and CXCL12-Cit_{8,12,20,41,47} failed to induce receptor internalization both on monocytes or lymphocytes. These results are in accordance with the binding experiments on CXCR4-transfected cells.

### Effect of Citrullination on CXCL12-Mediated Anti-HIV-1 Activity

Since CXCR4 is one of the main co-receptors for HIV binding on human leukocytes, the effect of citrullination of CXCL12 on its antiviral activity against the X4-tropic NL4.3 HIV-1 strain was investigated on lymphocytic MT-4 cells. CXCL12 inhibited HIV-infection with an IC₅O of 31 nM (FIG. 29). In contrast, CXCL12-Cit₈ had moderate antiviral activity, i.e. 23% inhibition of infection at 2 μM. CXCL12-Cit_{8,12,20} and _{CXCL}12-Cit_{8,12,20,41,47} failed to inhibit infection of lymphocytes with the NL4.3 HIV-1 strain at concentrations up to 2 μM.

### Example 4

### Citrullination of Other Cytokines and Chemokines by PAD

### A. Materials and Methods

Treatment with PAD

Recombinant human Mig/CXCL9, recombinant human CC chemokines HCC-1/CCL14, eotaxin-3/CCL26, TARC/CCL17, recombinant human cytokines IL-1 b, TNF-αor IL-6 (PeproTech) were incubated with PAD in 40 mM Tris-HCl, pH 7.6, 2 mM CaCl₂ at 37° C. The reaction was stopped by adding 0.1% TFA. Cytokines or chemokines treated with rabbit or human PAD were either desalted on C4 ZipTip (Millipore, Billerica, Mass.) prior to mass spectrometry or spotted on PVDF membranes (ProSorb; Applied Biosystems) prior to Edman degradation to determine the NH₂-terminal sequence of the proteins. The phenyl thiohydantoin (PTH) derivates of the individual amino acids were separated by RP-HPLC on a 0.8×250 mm PTH Column (Applied Biosystems) and the height of the peaks was used to calculate the relative amount of Arg converted to citrulline (Cit). The phenyl thiohydantoin derivate of Cit eluted between the derivates of Thr and Gly as described for Example 1.

### B. Results

Although PAD was not able to citrullinate IL-1β, as mentioned in example 1, incubations of other cytokines and chemokines with an Arg present in their NH₂-terminal region were performed with rabbit PAD, human PAD2 or PAD4. TNF-α was incubated with rabbit PAD at an enzyme/substrate molar ratio of 1/20. Conversion of Arg in positions 2 (Arg₂) and 6 (Arg₆) in the sequence of TNF-αwas determined by Edman degradation, Within 1 h, about 50% of Arg₂ was converted to Cit. For Arg₆, 50% modification was obtained after 90 min (Table IV).

TABLE IV
Citrullination of human TNF-α with PAD

| | |
|---|---|
| Incubation time | % conversion of Arg to Cit |

| (minutes) | Arg₂ | Arg₆ | E/S ratio |
|---|---|---|---|
| 10 | 33 | 26 | 1/20 |
| 30 | 41 | 32 | 1/20 |
| 60 | 53 | 19 | 1/20 |
| 90 | 86 | 58 | 1/20 |

Other CXC chemokines, CXCL5/ENA-78 and CXCL9/Mig, were also citrullinated by PAD. When incubated with rabbit PAD at an E/S of 1/20 for 90 minutes, the first NH₂-terminal Arg of both chemokines were practically completely deiminated (89.8 and 100%, respectively), whereas the second Arg were preserved (Table V). Though, after coincubation of CXCL5/ENA-78 with human PAD2 or PAD4, citrullination occurred slower in comparison with CXCL8 and CXCL10.

In addition to CXC chemokines, also CC chemokines were tested. incubation of HCC-1/CCL14 with human PAD4 for 90 at an E/S of 1/20 leaded to complete conversion of the first NH₂-terminal Arg (at position 6) in the HCC-1 protein into citrulline. This Arg is located in front of the peptide bond in HCC-1 that has to be cleaved in order to convert HCC-1 into its highly active HCC-1(9-74) form and may, in analogy with CXCL8/IL-8, protect it from proteolytic activation. As mentioned in example 1, CCL17/TARC and CCL26/eotaxin-3 were also citrullinated by PAD (Table II and V). However, CCL17 and CCL26 were citrullinated more slowly compared to CXCL8 and CXCL10. Human PAD4, but not human PAD2, citrullinated the first NH₂-terminal Arg in CCL17 with efficiency comparable to that in CXCL8. Moreover, the second Arg in CCL17 was also partially citrullinated by human PAD4 as well as by PAD2.

In addition to TNF-α, the cytokine IL-6 was also observed to be citrullinated by PAD. In contrast to IL-1β, the first NH2-terminal Arg of IL-6 was completely citrullinated after 17h of coincubation with rabbit PAD.

In conclusion, citrullination does not appear to be a general, naturally occurring phenomenon for all cytokines and occurs preferentially on a specific position in the cytokine or chemokine sequence which is in accordance with the posttranslational modification on natural CXCL8 and CXCL10 (vide supra).

TABLE V
Edman degradation and mass spectrometry after PAD incubation of immune mediators

| | NH₂-terminal | # | | | incubation | % | % | mass |
|---|---|---|---|---|---|---|---|---|
| mediator | sequence | Arg^{a} | enzyme^{b} | E/S^{c} | period^{d} | Cit^{e} | Cit^{f} | shift^{g} |
| CXCL5/ENA-78 | AGPAAAVLR^{h}ELR^{f}CVC- | 2 | rabbit | 1/20 | 90 | 89.8 | 0 | 1.0 |
| | | | PAD | | | | | |
| | | | hu PAD2 | 1/200 | 120 | 14.0 | 0 | 0.4 |
| | | | hu PAD4 | 1/200 | 120 | 35.1 | 0 | 0.6 |
| CXCL8/IL-8 | AVLPR^{h}SAKELRⁱCQC- | 5 | rabbit | 1/20 | 90 | 100 | 0 | 1.1 |
| | | | PAD | | | | | |
| | | | hu PAD2 | 1/200 | 120 | 88.0 | 0 | n.d. |
| | | | hu PAD4 | 1/200 | 120 | 94.8 | 0 | 1.0 |
| CXCL9/Mig | TPVVR^{h}KGRⁱCSC- | 5 | rabbit | 1/20 | 90 | 100 | 0 | n.d. |
| | | | PAD | | | | | |
| CXCL10/IP-10 | VPLSR^{h}TVRⁱCTC- | 6 | rabbit | 1/20 | 90 | 100 | 0 | 1.1 |
| | | | PAD | | | | | |
| | | | hu PAD2 | 1/200 | 120 | 85.7 | 0 | 2.0 |
| | | | hu PAD4 | 1/200 | 120 | 82.7 | 0 | n.d. |
| CXCL11/1-TAC | FPMFKR^{h}GRⁱCLC- | 5 | rabbit | 1/20 | 30 | 87.0 | n.d. | n.d. |
| | | | PAD | | | | | |
| CXCL12/SDF-1α | KPVSLSYR^{h}CPC- | 5 | rabbit | 1/20 | 90 | 100 | n.d. | n.d. |
| | | | PAD | | | | | |
| CCL14/HCC-1 | TESSSR^{h}GPYHPSECC- | 4 | hu PAD4 | 1/20 | 90 | 71.6 | n.d. | 1.3 |
| CCL17/TARC | AR^{h}GTNVGRⁱECC- | 7 | hu PAD2 | 1/200 | 120 | 24.8 | 22.6 | 0.8 |
| | | | hu PAD4 | 1/200 | 120 | 92.1 | 54.3 | 2.4 |
| CCL26/eotaxin-3 | TR^{h}GSDISKTCC- | 5 | hu PAD2 | 1/200 | 120 | 17.1 | n.d. | 0.2 |
| | | | hu PAD4 | 1/200 | 120 | 17.8 | n.d. | 0.2 |
| | | | hu PAD4 | 1/40 | 120 | 100 | n.d. | 1.3 |
| IL-1β | APVR^{h}SLNCTLRⁱDS- | 3 | rabbit | 1/10 | 17 h | 0 | 0 | n.d. |
| | | | PAD | | | | | |
| IL-6 | VPPGEDSKDVAAPHR^{h}QP- | 9 | rabbit | 1/10 | 17 h | 100 | n.d. | n.d. |
| | | | PAD | | | | | |
| TNF-α | VR^{h}SSSRⁱTPSDK- | 9 | rabbit | 1/20 | 90 | 86 | 58 | n.d. |
| | | | PAD | | | | | |

^{a}total number of Arg residues in entire sequence
^{b}natural purified rabbit skeletal muscle peptidylarginine deiminase (PAD) or human (hu) recombinant PAD
^{c}enzyme-substrate molar ratio
^{d}time period (min) of coincubation
^{e}% citrullination of the first NH₂-terminal Arg^{h} as determined by Edman degradation
^{f}% citrullination of the second NH₂-terminal Argⁱ as determined by Edman degradation
^{g}mass shift in Mr after comparison with Mr of intact mediator measured in parallel by ion trap mass
spectrometry
^{h}first NH₂-terminal Arg
ⁱsecond NH₂-terminal Arg

1-37. (canceled)

38. A cytokine having at least one of its Arg residues substituted by a citrulline residue or fragments, homologues or variants thereof comprising said citrulline residue(s).

39. The cytokine of claim 38 selected from the group of chemokines.

40. The cytokine of claim 38 selected from the group of CX₃C chemokines.

41. The cytokine of claim 38 wherein the first NH₂-terminally located Arg residue is substituted by a citrulline residue.

42. The cytokine of claim 38 wherein a maximum of 5 Arg residues are substituted by citrulline residues.

43. The cytokine of claim 38 selected from the group of CXCL8cit₅; CXCL10cit₅; CXCL11cit₆; CXCL12-Cit₈; CXCL12-Cit_{8,12,20}; or CXCL-Cit_{8,12,20,41,47}.

44. A pharmaceutical composition comprising a pharmaceutically acceptable carrier and a therapeutically effective amount of the cytokine of claim 38.

45. An antibody specifically directed against the cytokine of claim 38.

46. A method for treatment or prevention of inflammation related disorders in a patient, said method comprising administering to said patient the cytokine of claim 38.

47. The method of claim 46, wherein the inflammation related disorder is rheumatoid arthritis.

48. The use of the cytokine of claim 38 in a medicament for the mobilization of haematopoietic cells or endothelial cells in a patient.

49. A method to modulate the activity of a chemokine or cytokine, in which at least one Arg residue of said chemokine or cytokine is substituted by a citrulline residue.

50. The method of claim 49 wherein the first NH₂-terminally located Arg residue of said chemokine or cytokine is substituted by a citrulline residue.

51. A method for producing the cytokine of claim 38 comprising incubating said cytokine with the enzyme peptidylarginine deiminase.

52. A test kit for diagnosing patients comprising the antibody of claim 45.

53. A method for the determination of the predisposition of a patient to develop an inflammation related disorder comprising the determination of the presence of the cytokine of claim 38 in a biological sample derived from said patient.

54. The method of claim 53 wherein the presence of the cytokine is determined using the antibody of claim 45.

55. A method to enhance the stability of a biologically active protein, in which at least one Arg or Lys residue of said biologically active protein is substituted by a citrulline residue.

56. The method of claim 55, wherein the biologically active protein is selected from the group consisting of cytokines or chemokines.
